(19) United States
(12) Patent Application Publication
(10) Pub. No.: US 2022/0089718 A1
(43) Pub. Date: Mar. 24, 2022

(54) CHIMERIC ANTIGEN RECEPTORS WITH MODIFIED LINKER DOMAINS AND USES THEREOF

(71) Applicant: BIOSCEPTRE (UK) LIMITED, Cambridge (GB)

(72) Inventors: Kanchana Veronika Bandara, Adelaide (AU); Justin Taylor Coombs, Wattle Park (AU); Timothy John Sadlon, Maylands (AU); Simon Charles Barry, Longwood (AU); Jade Hui Yu Foeng, Tusmore (AU); Shaun Reuss McColl, Myrtle Bank (AU)

(21) Appl. No.: 17/057,060

(22) PCT Filed: May 20, 2019
(86) PCT No.: PCT/AU2019/050487

§ 371 Date: Nov. 19, 2020

(30) Foreign Application Priority Data
May 21, 2018 (AU) 2018901782

(51) Int. Cl. C07K 16/28; A61K 35/17; C07K 14/705; C07K 14/725; A61P 35/00

(52) U.S. Cl. CPC C07K 16/28; A61K 35/17; C07K 14/70521; C07K 2317/53; A61P 35/00; C07K 2319/03; C07K 14/7051

(57) ABSTRACT

The present invention relates to an optimized chimeric antigen receptor (CAR), and genetically modified cells expressing the same, that can target a diverse range of cancer types. More specifically, the CAR has an optimized linker length that facilitates the targeting and lyse of a wide range of cancer cell types by CAR expressing T cells.

### PRIORITY CLAIM

This application claims priority from Australian provisional application number 2018901782 filed on 21 May 2018, the entire contents of which is herein incorporated by way of this reference.

### TECHNICAL FIELD

The present invention relates to chimeric antigen receptors, immune cells expressing chimeric antigen receptors and methods of using chimeric antigen receptors for the prevention and/or treatment of cancer.

### BACKGROUND OF INVENTION

The immune system has highly evolved and specific mechanisms that protect organisms from a range of pathologies. Amongst these mechanisms is the detection and elimination of unwanted pathogens such as bacterial infections, virally infected cells, and importantly, mutated cells that may cause malignant neoplasia (cancer). The ability of the immune system to prevent the formation and growth of cancers is dependent on the ability of the cells of the immune system to distinguish between a ‘healthy’ cell and a ‘diseased’ (e.g. neoplastic or pre-neoplastic) cell. This is achieved by recognition of cell markers (antigens) that are indicative of the transition of a cell from a healthy state to a diseased state.

There have been many attempts to develop immunotherapeutic approaches to treat cancer by manipulating or directing the immune system to target cells expressing cancer cell antigens. Immunotherapeutic approaches have largely centred on either exploiting the humoral immune system by utilising isolated or engineered antibodies or, more recently, the cellular arm of the immune system.

One means to perform cellular immunotherapy for the treatment of cancer utilises T lymphocytes isolated from tumours, which are expanded ex vivo before re-administration to the patient. While this approach has provided some promise and efficacy, there are many technical challenges associated with this approach. The heterogeneous nature of the isolated tumour derived T cells, and the challenges expanding cells ex vivo, may result in an expanded population containing only a small number of cancer antigen-specific T cells. As a result, the efficacy of this method is unpredictable and variable.

In order to address some of the shortfalls related to the use of ex vivo expanded tumour-isolated T cells, chimeric antigen receptor (CARs or artificial T cell receptors) began to be developed in the late 1980s. Chimeric antigen receptors combine an extracellular region, specific for a desired antigen, to an intracellular signalling region, resulting in an antigen-specific receptor that can induce T cell function.

Transformation of isolated T cells with CARs results in a population of T cells that are specific for a given antigen. These cells combine the antigen-specificity of an antigen binding molecule with the lytic capacity and self-renewal of an endogenous T cell. As a result, large populations of antigen-specific T cells can be generated and administered to a patient.

To date, the development and implementation of CAR T cell therapies has been limited. Primarily, CAR T cells have been used to treat haematological cancers such as B-cell lymphomas. Treatment of such conditions using CAR T cells directed against the B cell marker CD19 has resulted in up to an 80% objective response rate, and greater than a 50% complete response rate in stage IV lymphoma patients.

However, despite the success of CAR T cell therapies in treatment of haematological cancers, their use in other cancer types has been limited. There are many reasons that CAR T cells have not been successful in treatment of other cancer types, particularly solid tumours. These reasons include T cell access to solid tumours, the hostile and immuosuppressive microenvironment within solid tumours and, importantly, difficulties in developing CAR-T cells that target and attack cancer cells expressing solid-tumour specific antigens.

Even when a tumour-specific antigen is identified, the ability to generate a CAR T cell that effectively targets solid-tumour cells, as well as targeting a diverse range of cancer types, is difficult.

It is therefore apparent that there is a need for the development of a CAR that targets a tumour-associated antigen, which is selectively expressed by cancerous cells, and induces a response in cells transduced with the CAR.

The discussion of documents, acts, materials, devices, articles and the like is included in this specification solely for the purpose of providing a context for the present invention. It is not suggested or represented that any or all of these matters formed part of the prior art base or were common general knowledge in the field relevant to the present invention as it existed before the priority date of each claim of this application.

### Summary of Invention

The present invention is predicated, in part, on the recognition that the ability of a CAR directed against a dysfunctional P2X7 receptor to recognise its antigen varies depending on the length of the linker domain between the antigen-recognition domain and the transmembrane domain of the CAR. Consequently, the efficacy of CAR-expressing immune cells to target cells expressing a dysfunctional P2X7 receptor is influenced by the length of the linker domain between the antigen-recognition domain and the transmembrane domain.

Accordingly, the present invention provides a chimeric antigen receptor including an antigen-recognition domain recognising a dysfunctional P2X7 receptor, a transmembrane domain and a linker domain, wherein the linker domain consists of between 12 to 228 amino acids.

In some embodiments, the present invention provides a chimeric antigen receptor including an antigen-recognition domain recognising a dysfunctional P2X7 receptor, a transmembrane domain and a linker domain, wherein the linker domain consists of between 30 to 228 amino acids.

In some embodiments, the linker domain consists of 50 to 200 amino acids, or 70 to 180 amino acids, or 90 to 160 amino acids, or 110 to 130 amino acids, or 115 to 125 amino acids, or 117 to 121 amino acids. In some embodiments, the linker domain consists of about 119 amino acids.

In some embodiments, the chimeric antigen receptor includes a linker domain that includes an amino acid sequence homologous to an immunoglobulin hinge region of IgG, IgD, IgA, or a constant heavy (CH) 2 region of IgM or IgE, or a functional variant thereof having at least 50%, 60%, 70%, 80%, 90%, 93%, ₉₆% ₒᵣ 99% sequence identity.

In some embodiments, the linker domain of the chimeric antigen receptor includes an amino acid sequence homologous to a hinge region from an IgG isotype immunoglobulin, or a functional variant thereof having at least 50%, 60%, 70%, 80%, 90%, 93%, 96% or 98% sequence identity. Preferably, the linker domain includes an amino acid sequence homologous to the hinge region of an IgG1, IgG2 or IgG4 subclass antibody, or a functional variant thereof having at least 50%, 66%, 73%, 75%, 80%, 83%, 86%, 91% or 93% sequence identity.

In some embodiments, the linker domain of the chimeric antigen receptor includes an amino acid sequence homologous to a hinge region from an IgG isotype immunoglobulin and includes a CXXC motif, wherein “C” is a Cysteine and “X” is any amino acid. In some embodiments the CXXC motif is selected from the group consisting of CPPC, CPRC or CPSC.

In some embodiments, the linker domain of the chimeric antigen receptor includes one or more amino acid sequences homologous to a CH region of an immunoglobulin or a functional variant thereof having at least 50%, 60%, 70%, 80%, 90%, 95%, 98% or 99% sequence identity. In some embodiments, the amino acid sequence homologous to the CH region is homologous to one or more of a CH1 region, a CH2 region, a CH3 region or a CH4 region of an immunoglobulin, or has 50%, 60%, 70%, 80%, 90%, 95%, 98% or 99% A sequence identity with said CH regions.

In some embodiments, the linker domain of the chimeric antigen receptor includes one or more amino acid sequence(s) homologous to one or more of a CH2 region or a CH3 region of an IgG isotype immunoglobulin or has 50%, 60%, 70%, 80%, 90%, 95%, 98% or 99% sequence identity with said CH2 or CH3 region(s).

In some embodiments, the linker domain of the chimeric antigen receptor includes one or more immunoglobulin hinge region(s) and/or one or more CH region(s) of an immunoglobulin. In some embodiments, the linker domain of the chimeric antigen receptor consists of a sequence homologous to an immunoglobulin hinge region and a CH region, preferably a CH2 region or CH3 region. In some embodiments the hinge region, CH2 region or CH3 region are from an IgG isotype immunoglobulin. In some embodiments, the hinge region, CH2 region or CH3 region are from the IgG4 subclass.

In some embodiments, the linker domain consists of an IgG hinge region and one or more CH region(s) of an immunoglobulin. In some embodiments the linker domain consists of an IgG hinge region and a CH2 or CH3 region of an immunoglobulin.

In some embodiments, the linker domain of the chimeric antigen receptor includes an amino acid sequence according to any one of SEQ ID NOs: 9 to 17, or a functional variant having at least 50%, 60%, 70%, 80%, 90%, 93% or 96% sequence identity. Preferably, the chimeric antigen receptor includes an amino acid sequence according to SEQ ID NOs: 9 to 13, or a functional variant thereof having at least 50%, 60%, 70%, 80%, 90%, 93% or 96% sequence identity.

In some embodiments, the linker domain of the chimeric antigen receptor does not comprise an amino acid sequence in the linker domain that substantially binds with an Fc receptor.

In some embodiments, the chimeric antigen receptor according to the present invention, when expressed in a CD8+ T cell, has a cytotoxicity in vitro against target cells expressing a dysfunctional P2X7 receptor of at least 20%, at a ratio of T cells: target cells of 30:1 or greater. In some embodiments, the target cells expressing a dysfunctional P2X7 receptor are cancer cells.

In some embodiments of the present invention, the antigen-recognition domain of the chimeric antigen receptor, recognises an epitope associated with an adenosine triphosphate (ATP)-binding site of the P2X7 receptor. In some embodiments, the dysfunctional P2X7 receptor has a reduced capacity to bind ATP compared to an ATP-binding capacity of a fully functional P2X7 receptor. In some embodiments, the dysfunctional P2X7 receptor has a conformational change that renders the receptor dysfunctional. In some embodiments, the conformational change is a change of an amino acid from a trans-conformation to a cis-conformation; preferably, the conformational change is the proline at amino acid position 210 of the dysfunctional P2X7 receptor.

In some embodiments, the antigen-recognition domain of the chimeric antigen receptor recognises an epitope that includes one or more amino acid residues spanning from glycine at amino acid position 200 to cysteine at amino acid position 216 of the dysfunctional P2X7 receptor. In some embodiments, the antigen-recognition domain of the chimeric antigen receptor recognises an epitope that includes proline at amino acid position 210 of the dysfunctional P2X7 receptor.

In some embodiments, the antigen-recognition domain of the chimeric antigen receptor comprises an amino acid sequence homologous to the amino acid sequence of an antigen binding region of an antibody. In some embodiments, the antigen-recognition domain of chimeric antigen receptor comprises an amino acid sequence homologous to the amino acid sequence of a domain region comprising at least 3 complementarity-determining regions (CDRs) of the variable heavy or variable light chain of an antibody that binds to a dysfunctional P2X7 receptor, or sequence homology to a single-chain variable fragment of an antibody (scFv) that binds to a dysfunctional P2X7 receptor.

In some embodiments, the chimeric antigen receptor of the present invention includes a transmembrane domain which comprises all or part of the transmembrane domain of CD3, CD4, CD8 or CD28; preferably, CD8 or CD28; more preferably, CD28.

The present invention further provides the use of a chimeric antigen receptor as described above, when expressed in an immune cells, for treating a cancer. In some embodiments, the immune cell is a leukocyte, in some embodiments, the immune cell is a Peripheral Blood Mononuclear Cell (PBMC). In some embodiments, the immune cell is a lymphocyte. In some embodiments, the immune cell is a T cell. In some embodiments, the immune cell is an alpha beta (αβ) T cell. In some embodiments, the immune cell is a gamma delta (γδ) T cell. In some embodiments, the immune cell is a virus-specific T cell. In some embodiments, the T cell is a CD3+ T cell. In some embodiments, the T cell is a CD4+ T cell. In some embodiments, the T cell is a CD8+ T cell. In some embodiments, the immune cell is a natural killer cell. In some embodiments, the immune cell is a natural killer T cell. In some embodiments, the cancer is a solid cancer.

The present invention further provides a nucleic acid molecule, or a nucleic acid construct, including a nucleotide sequence encoding the chimeric antigen receptor described above.

The present invention further provides a genetically modified cell including the chimeric antigen receptor, nucleic acid molecule, or nucleic acid construct as described above. In some embodiments, the genetically modified cell is a leukocyte, in some embodiments, the genetically modified cell is a Peripheral Blood Mononuclear Cell (PBMC). In some embodiments, the genetically modified cell is a lymphocyte. In some embodiments, the genetically modified cell is a T cell. In some embodiments, the genetically modified cell is an alpha beta (αβ) T cell. In some embodiments, the genetically modified cell is a gamma delta (γδ) T cell. In some embodiments, the genetically modified cell is a virus-specific T cell. In some embodiments, the T cell is a CD4+ T cell. In some embodiments, the T cell is a CD8+ T cell. In some embodiments, the genetically modified cell is a natural killer cell. In some embodiments, the genetically modified cell is a natural killer T cell.

The present invention further provides use of a genetically modified cell as described above for treating cancer. Furthermore, the invention provides a method of killing a cell expressing a dysfunctional P2X7 receptor, the method including exposing the cell expressing a dysfunctional P2X7 receptor to a cell including a chimeric antigen receptor, nucleic acid molecule or nucleic acid construct, as described above. In some embodiments, the invention provides a method of killing a cell expressing a dysfunctional P2X7 receptor, the method including exposing the cell expressing a dysfunctional P2X7 receptor to a genetically modified cell as described above. In some embodiments, the cells expressing a dysfunctional P2X7 receptor is a cancer cell.

In some embodiments, the cancer cell is a solid cancer cell. In some embodiments, the cancer cell is selected from the group consisting of: brain cancer cell, oesophageal cancer cell, mouth cancer cell, tongue cancer cell, thyroid cancer cell, lung cancer cell, stomach cancer cell, pancreatic cancer cell, kidney cancer cell, colon cancer cell, rectal cancer cell, prostate cancer cell, bladder cancer cell cervical cancer cell, epithelial cell cancers, skin cancer cell, leukaemia cell, lymphoma cell, myeloma cell, breast cancer cell, ovarian cancer cell, endometrial cancer cell and testicular cancer cell. In some embodiments, the cancer cell is selected from the group consisting of: a breast cancer cell, a prostate cancer cell, a glioblastoma cancer cell, an ovarian cancer cell, or a melanoma cancer cell. In some embodiments, the cancer cell is from a metastatic cancer. In some embodiments, the cancer cell is from, or is within, a patient who has stage III cancer, or is stage IV cancer

In some embodiments, the genetically modified cell is autologous to the cell expressing a dysfunctional P2X7 receptor. In some embodiments, the cell expressing a dysfunctional P2X7 receptor is within the body of a subject.

The present invention also provides a pharmaceutical composition including a genetically modified cell including a chimeric antigen receptor, a nucleic acid molecule or a nucleic acid construct as described above and a pharmaceutically acceptable carrier or excipient.

In at least some embodiments, the present invention provides a lentiviral vector comprising a nucleic acid encoding a chimeric antigen receptor as described herein.

Further, the present invention provides a use of a chimeric antigen receptor, lentiviral vector, genetically modified cell or a nucleic acid as described herein for the prevention or treatment of cancer. In at least some embodiments, there is provided a use of the chimeric antigen receptor, lentiviral vector, genetically modified cell or a nucleic acid in the manufacture or preparation of a medicament for use in the prevention or treatment of cancer.

In at least some embodiments, the medicament is used for the prevention or treatment of a solid cancer cell. In some embodiments, the medicament is used for the prevention or treatment of a cancer cell selected from the group consisting of: brain cancer cell, oesophageal cancer cell, mouth cancer cell, tongue cancer cell, thyroid cancer cell, lung cancer cell, stomach cancer cell, pancreatic cancer cell, kidney cancer cell, colon cancer cell, rectal cancer cell, prostate cancer cell, bladder cancer cell cervical cancer cell, epithelial cell cancers, skin cancer cell, leukaemia cell, lymphoma cell, myeloma cell, breast cancer cell, ovarian cancer cell, endometrial cancer cell and testicular cancer cell. In some embodiments, the medicament is used for the prevention or treatment of a cancer cell selected from the group consisting of: a breast cancer cell, a prostate cancer cell, a glioblastoma cancer cell, an ovarian cancer cell, or a melanoma cancer cell. In some embodiments, the cancer cell is from a metastatic cancer. In some embodiments, the cancer cell is from, or is within, a patient who has stage III cancer, or is stage IV cancer

### BRIEF DESCRIPTION OF DRAWINGS

FIG. 1: Schematic of CAR constructs in accordance with an embodiment of the present invention.

FIG. 2: Alignment of the hinge region of the IgG subtype antibodies and the mutated hinge region of an exemplary embodiment of the present invention.

FIG. 3: Alignment of IgG1, IgG2, IgG4 antibodies and the mutated hinge region of an exemplary embodiment of the present invention.

FIG. 4: Alignment of the CH2 regions of the IgG subtype antibodies and the CH2 region of an exemplary embodiment of the present invention.

FIG. 5: Alignment of the CH3 regions of the IgG subtype antibodies and the CH3 region of an exemplary embodiment of the present invention.

FIG. 6: epHIV-7.2 Lentiviral vector including CNA1004 CAR.

FIG. 7: Scatter plots of EGFRt and Fc expression on CAR transduced CD4+ cells.

FIG. 8: Scatter plots of EGFRt and Fc expression on CAR transduced CD8+ cells.

FIG. 9: Scatter plots of EGFRt and Fc expression on isolated and expanded CAR transduced CD4+ cells

FIG. 10: Scatter plots of EGFRt and Fc expression on isolated and expanded CAR transduced CD8+ cells

FIG. 11: Killing assays of CAR transduced CD8+ T cells against various target cell lines.

FIG. 12: Killing assays of CAR transduced CD8+ T cells against various target cell lines.

FIG. 13: Scatter plot of CD4 and CD8 expression on T cells transduced with CNA1003 CAR according to Protocol 2, as well as histograms of EGFR expression.

FIG. 14: Killing assay of CD3+ CNA1003 CAR T cells against various cancer cell lines.

FIG. 15: Killing assay of CD3+ CNA1003 CAR T cells against various cancer cell lines.

FIG. 16: Killing assay of CD8+ CNA1003 CAR T cells against various cancer cell lines.

FIG. 17: Killing assay of CD4+ CNA1003 CAR T cells against various cancer cell lines.

FIG. 18: Cytokine secretion assays of CAR transduced CD4+ T cells against various target cell lines

FIG. 19: Cytokine secretion assays of CAR transduced CD4+ T cells against various target cell lines

FIG. 20: BLIV vector as used in an additional embodiment of the invention.

FIG. 21: Killing assay assessing the effector function of CD8+ T cell expressing a short hinge and long hinge BLIV-CAR.

FIG. 22: Killing assay of CAR constructs having varying antigen-recognition domains, against variable cancer cell lines.

FIG. 23: CD3+ CNA1003 CAR T cell function in an in vivo xenograft model of prostate cancer.

FIG. 24: Percentage of live CD4+ and CD8+ tumour infiltrating CNA1003 CAR T cells with a single and double dose of CD3+ cells.

FIG. 25: Cytokine secretion and activation profile of CD3+CD4+ tumour infiltrating CNA1003 CAR T cells.

FIG. 26: Cytokine secretion and activation profile of CD3+CD8+ tumour infiltrating CNA1003 CAR T cells

FIG. 27: CD8+ CNA1003 CAR T cell function in an in vivo xenograft model of prostate cancer.

FIG. 28: Cytokine profile and activation profile of the CD8+ tumour infiltrating CNA1003 CAR T cells.

FIG. 29: Tumour growth and size in a mouse xenograft prostate cancer model administered with varying doses of CNA1003 CART cells.

FIG. 30: Total and percentage of live CD3+ tumour infiltrating CNA1003 CAR T cells from mice administered with a single dose or a double dose of 1×10⁷ or 2×10⁷ CD3+ CAR T cells and phenotype analysis of tumour infiltrating CAR T cells.

FIG. 31: The expression of cytotoxic effector molecules granzyme b and perforin by CD4+ and CD8+ tumour infiltrating CAR T cells.

FIG. 32: CD8+ CNA1003 CAR T cell function in an in vivo xenograft model of breast cancer and quantification of lung metastatic nodules.

### DETAILED DESCRIPTION

The nucleotide and polypeptide sequences referred to herein are represented by a sequence identifier number (SEQ ID NO:). A summary of the sequence identifiers is provided in Table 1. A sequence listing is also provided as part of the specification.

TABLE 1
Sequences Descriptions

| Sequence | |
|---|---|
| Identifier | Sequence |
| SEQ ID NO: 1 | Human P2X₇ receptor mRNA sequence |
| SEQ ID NO: 2 | Human P2X₇ receptor coding (cDNA) sequence |
| SEQ ID NO: 3 | Human P2X₇ receptor amino acid sequence |
| SEQ ID NO: 4 | Pep2-2-1 amino acid sequence |
| SEQ ID NO: 5 | Pep2-2-1 nucleic acid sequence |
| SEQ ID NO: 6 | Pep2-2-3 amino acid sequence |
| SEQ ID NO: 7 | Pep2-472-2 amino acid sequence |
| SEQ ID NO: 8 | Pep2-2-12 amino acid sequence |
| SEQ ID NO: 9 | IgG1 hinge amino acid sequence |
| SEQ ID NO: 10 | IgG2 hinge amino acid sequence |
| SEQ ID NO: 11 | IgG3 hinge amino acid sequence |
| SEQ ID NO: 12 | IgG4 hinge amino acid sequence |
| SEQ ID NO: 13 | IgG4 hinge (mutated) amino acid sequence |
| SEQ ID NO: 14 | IgA1 hinge amino acid sequence |
| SEQ ID NO: 15 | IgD hinge amino acid sequence |
| SEQ ID NO: 16 | IgM common heavy (CH) 2 amino acid sequence |
| SEQ ID NO: 17 | IgE CH2 amino acid sequence |
| SEQ ID NO: 18 | CD28 hinge amino acid sequence |
| SEQ ID NO: 19 | CD8 hinge (1) amino acid sequence |
| SEQ ID NO: 20 | CD8 hinge (2) amino acid sequence |
| SEQ ID NO: 21 | CD8b hinge (1) amino acid sequence |
| SEQ ID NO: 22 | CD8b hinge (2) amino acid sequence |
| SEQ ID NO: 23 | CD7 hinge amino acid sequence |
| SEQ ID NO: 24 | CD4 hinge amino acid sequence |
| SEQ ID NO: 25 | PolyG/S amino acid sequence |
| SEQ ID NO: 26 | IgG1 heavy domain amino acid sequence |
| SEQ ID NO: 27 | IgG2 heavy domains amino acid sequence |
| SEQ ID NO: 28 | IgG3 heavy domains amino acid sequence |
| SEQ ID NO: 29 | IgG4 heavy domains amino acid sequence |
| SEQ ID NO: 30 | IgG1 CH2 amino acid sequence |
| SEQ ID NO: 31 | IgG1 CH3 amino acid sequence |
| SEQ ID NO: 32 | IgG2 CH2 amino acid sequence |
| SEQ ID NO: 33 | IgG2 CH3 amino acid sequence |
| SEQ ID NO: 34 | IgG3 CH2 amino acid sequence |
| SEQ ID NO: 35 | IgG3 CH3 amino acid sequence |
| SEQ ID NO: 36 | IgG4 CH2 amino acid sequence |
| SEQ ID NO: 37 | IgG4 CH3 amino acid sequence |
| SEQ ID NO: 38 | CNA1002 linker domain amino acid sequence |
| SEQ ID NO: 39 | CNA1003 linker domain amino acid sequence |
| SEQ ID NO: 40 | CNA1004 linker domain amino acid sequence |
| SEQ ID NO: 41 | BLIV CAR Short hinge amino acid sequence |
| SEQ ID NO: 42 | CD3 zeta amino acid sequence |
| SEQ ID NO: 43 | CD3 epsilon amino acid sequence |
| SEQ ID NO: 44 | CD3 gamma amino acid sequence |
| SEQ ID NO: 45 | CD3 delta amino acid sequence |
| SEQ ID NO: 46 | CD3 zeta CAR domain intracellular |
| | amino acid sequence |
| SEQ ID NO: 47 | FCεR1 amino acid sequence |
| SEQ ID NO: 48 | FCγR1 amino acid sequence |
| SEQ ID NO: 49 | 4-1BB CAR intracellular amino acid sequence |
| SEQ ID NO: 50 | CNA1003 CAR complete amino acid sequence |
| SEQ ID NO: 51 | CNA1003 CAR amino acid sequence |
| | (minus T2 and EGFRt sequence) |
| SEQ ID NO: 52 | Nucleic acid sequence encoding for SEQ ID NO: 50 |
| SEQ ID NO: 53 | Nucleic acid sequence encoding for SEQ ID NO: 51 |
| SEQ ID NO: 54 | CNA1002 CAR complete amino acid sequence |
| SEQ ID NO: 55 | Nucleic acid sequence encoding for SEQ ID NO: 54 |
| SEQ ID NO: 56 | CNA1004 CAR complete amino acid sequence |
| SEQ ID NO: 57 | Nucleic acid sequence encoding for SEQ ID NO: 56 |
| SEQ ID NO: 58 | BLIV-CAR short linker CAR nucleotide sequence |
| SEQ ID NO: 59 | BLIV-CAR long linker CAR nucleotide sequence |
| SEQ ID NO: 60 | BLIV-CAR short linker CAR amino acid sequence |
| SEQ ID NO: 61 | BLIV-CAR long linker CAR amino acid sequence |
| SEQ ID NO: 62 | Amino acid sequence of truncated Epithelial |
| | Growth Factor Receptor (EGFRt) |
| SEQ ID NO: 63 | BLIV-CAR long linker domain amino acid |
| SEQ ID NO: 64 | CNA1103 antigen-recognition domain |
| SEQ ID NO: 65 | CNA1203 antigen-recognition domain |
| SEQ ID NO: 66 | CNA1303 antigen-recognition domain |
| SEQ ID NO: 67 | CNA1403 antigen-recognition domain |
| SEQ ID NO: 68 | CNA1503 antigen-recognition domain |
| SEQ ID NO: 69 | Coupling region of CNA1303, CNA1403 |
| | and CNA1503 binding domains |

The present invention is predicated, in part, on the recognition by the Inventors that the ability of a CAR to recognise a dysfunctional P2X7 receptor varies depending on the length of the linker domain between the antigen-recognition domain and the transmembrane domain of the CAR. Consequently, the efficacy of CAR-expressing immune cells to target cells expressing a dysfunctional P2X7 receptor is influenced by the length of the linker domain linking the antigen-recognition domain to the transmembrane domain. Specifically, the ability of CAR-expressing immune cells to target and kill a wide range of cancer cell types is influenced by the linker length.

As known in the art, chimeric antigen receptors (CARs) are artificially constructed proteins that upon expression on the surface of a cell can induce an antigen-specific cellular response. A CAR includes at a minimum three domains; the first domain being an extracellular antigen-recognition domain that specifically recognises an antigen, or more specifically an epitope portion, or portions, of an antigen; the second domain being an intracellular signalling domain that is capable of inducing, or participating in the induction, of an intracellular signalling pathway; and the third being a transmembrane domain that traverses the plasma membrane and bridges the extracellular antigen-recognition domain and the intracellular signalling domain.

The combination of the first two domains determines the antigen specificity of the CAR and the ability of the CAR to induce a desired cellular response, the latter of which is also dependent on the host cell of the CAR. For example, the activation of a CAR expressed in a T-helper cell, and having a signalling domain comprising a CD3 activation domain, may, once activated by encountering its cognate antigen, induce the CD4+ T-helper cell to secrete a range of cytokines. In a further example, the same CAR when expressed in a CD8+ cytotoxic T cell, once activated by a cell expressing the cognate antigen, may induce the release of cytotoxins that ultimately lead to the induction of apoptosis of the antigen-expressing cell.

The third domain (the transmembrane domain) may comprise a portion of, or may be associated with, the signalling domain of the CAR. The transmembrane domain is typically one or more hydrophobic helices, which spans the lipid bilayer of a cell and embeds the CAR within the cell membrane. The transmembrane domain of the CAR can be one determinant in the expression pattern of the CAR when associated with a cell. For example, using a transmembrane domain associated with a CD3 co-receptor can permit expression of the CAR in naïve T cells, amongst others, whilst use of a transmembrane domain from a CD4 co-receptor may direct expression of a CAR in T-helper cells. Use of the CD8 co receptor transmembrane domain may direct expression in cytotoxic T lymphocytes (CTLs), while the CD28 transmembrane domain may permit expression in both CTLs and T helper cells and can assist in stabilising the CAR.

A further component, or portion, of a chimeric antigen receptor may be a linker domain. The linker domain spans from the extracellular side of the transmembrane domain to the antigen-recognition domain, thereby linking the antigen-recognition domain to the transmembrane domain. Typically, in the art, the linker domain is considered as an optional domain, as some CARs function without a linker domain.

While not wanting to be bound by theory, it is hypothesised that the effector function of a T cell is dependent on the formation of an appropriately sized synapse between the T cell and its target cell. Typically, when a T cell recognises an antigen via its T cell receptor (TCR), the epitope of the antigen is being presented by a Major Histocompatibility Complex (MHC) molecule (specifically MHC class I for CD8+ T cells, and MHC class II for CD4+ T cells). Consequently, the distance between the T cell and the target cell (the synaptic distance) is constant (this is dictated by the length of the TCR and MHC molecule). However, this is not the case for CAR T cell.

The epitope recognised by a given CAR T cell will vary depending on the size and structure of the target molecule, the location of the epitope on the target molecule and the nature of the chimeric antigen receptor, particularly the antigen recognition domain. Further, depending on the location of the epitope on the target molecule, the chimeric antigen receptor may need a degree of flexibility to allow orientation of the antigen recognition domain to appropriately interact with and recognise the target molecule.

Consequently, it can be beneficial to include a linker domain in a CAR as the linker domain may provide flexibility to the antigen recognition domain of the CAR, to permit the necessary orientation of the antigen-recognition domain, and regulate the immune synapse distance.

The present inventors have recognised that the function of a chimeric antigen receptor directed against a dysfunctional P2X7 receptor is optimized when the linking domain, which connects the antigen-recognition domain to the transmembrane domain, is between 12 to 228 amino acids, or preferably between 30 to 228 amino acids. Resultantly, the optimized chimeric antigen receptor is able to target a wide range of cells types expressing a dysfunctional P2X7 receptor. Preferably, the target cells are cancer cells and the optimized chimeric antigen receptor (when expressed on an immune cell) can target a wide range of cancer cell types. This is particularly advantageous as the dysfunctional P2X7 is expressed by a broad range of malignancies, and therefore immune cells expressing the optimized chimeric antigen receptor of the present invention can target a diverse range of cancers.

Consequently, the present invention provides a chimeric antigen receptor including an antigen-recognition domain recognising a dysfunctional P2X7 receptor, a transmembrane domain and a linker domain, wherein the linker domain consists of between 12 to 228 amino acids. In some embodiments, the linker domain consists of between 30 to 228 amino acids.

### Antigen Recognition Domain

A chimeric antigen receptor, which targets cells expressing a dysfunctional P2X7 receptor, is described in the international publication WO2017/041143, the entire disclosure of which is incorporated by way of this reference.

The P2X7 receptor (purinergic receptor P2X, ligand-gated ion channel, 7) is an ATP-gated ion channel that is expressed in a number of species including humans. The receptor is encoded by a gene, the official symbol of which is represented by P2RX7. The gene has also been referred to as P2X purinoceptor 7, ATP receptor, P2Z receptor, P2X7 receptor, and purinergic receptor P2X7 variant A. For the purposes of the present disclosure, the gene and encoded receptor will be referred to herein as P2X7 and P2X7, respectively.

The mRNA, coding (cDNA), and amino acid sequences of the human P2X7 gene are set out in SEQ ID NOs: 1 to 3, respectively. The mRNA and amino acid sequences of the human P2X7 gene are also represented by GenBank Accession Numbers NM_002562.5 and NP_002553.3, respectively. The P2X7 gene is at least partially conserved in chimpanzee, Rhesus monkey, dog, cow, mouse, rat, pig, chicken, zebrafish, and frog. Further details of the P2X7 gene in human and other species may be accessed from the GenBank database at the National Centre for Biotechnology Information (NCBI) (www.ncbi.nlm.nih.gov). For example, the Gene ID number for human P2X7 is 5027, for chimpanzee is 452318, for monkey is 699455, for canine is 448778, for cow is 286814, for mouse is 18439, for zebrafish is 387298, and for frog is 398286. Furthermore, at least 73 organisms have orthologs with the human P2X7 gene.

Further details regarding the P2X7 gene in humans and other species can also be found at the UniGene portal of the NCBI (for example see UniGene Hs. 729169 for human P2X7—http://www.ncbi.nlm.nih.gov/UniGene/clust.cgi?UGID=4540770&TAXID=9606&SEARCH). Alternatively, details of the nucleotide and amino acid sequences for the P2X7 gene can be accessed from the UniProt database (www.uniprot.org) wherein the UniProt ID for the human P2X7 gene is Q99572. The contents of the GenBank and UniProt records are incorporated herein by reference.

The P2X7 receptor is formed from three protein subunits (monomers), wherein in the native receptor in humans at least one of the monomers has an amino acid sequence set forth in SEQ ID NO: 3. It is to be understood that a “P2X7 receptor” as referred to herein also includes naturally occurring variations of the receptor including splice variants, naturally occurring truncated forms and allelic variants of the receptor. A P2X7 receptor may also include subunits that have a modified amino acid sequence, for example those including truncations, amino acid deletions or modifications of the amino acid set forth in SEQ ID NO: 3.

A “variant” of the P2X7 gene or encoded protein may exhibit a nucleic acid or an amino acid sequence, respectively, that is at least 80% identical, at least 90% identical, at least 95% identical, at least 98% identical, at least 99% identical, or at least 99.9% identical to a native P2X7 receptor, for example.

The P2X7 receptor is activated by the binding of ATP to the ATP-binding site of the receptor. This leads to the rapid opening (within milliseconds) of a channel that selectively allows for movement of small cations across the membrane. After a short period of time (within seconds) a large pore is formed in the membrane of a cell that allows for permeation of the cell membrane by molecules up to 900 Da in size. This pore formation ultimately leads to depolarization of the cell and in many cases cytotoxicity and cell death. This role leads to a belief that the P2X7 receptor is involved in apoptosis in a variety of cell types.

A decrease, or loss, in function of the P2X7 receptor can lead to a cell that is comparatively resistant to induced apoptosis. In many cases this resistance to apoptosis is critical in the transition of a normal ‘healthy’ cell to a mutated pre-cancerous or cancerous cell. Consequently, the ability to target cells that have a decreased function, or a loss of function, of the P2X7 receptor provides possible target for cancer therapy.

Accordingly, the chimeric antigen receptor of the invention recognises a dysfunctional P2X7 receptor. As used throughout the specification the term “dysfunctional”, with reference to the P2X7 receptor includes a decrease in function of the receptor with respect to its comparatively normal function in a comparable cell. In some embodiments, the function of P2X7 receptor may be decreased by at least 1° A, 5%, 10%, 20%, 30%, 40%, 50%, 60%, 70%, 80%, 90% or greater than 99%. In some embodiments, the term “dysfunctional” may include a P2X7 receptor that is non-functional.

Any change in the wild-type or native form of the P2X7 receptor that leads to a dysfunctional receptor is encompassed herein. For example, the dysfunctional receptor may be the result of a mutation or alteration in one or more amino acids of the receptor that are associated with ATP binding to the receptor. In effect, the P2X7 receptor is dysfunctional as it has a reduced capacity to, or cannot, bind ATP at the ATP-binding site. In this instance, the antigen-recognition domain of the chimeric antigen receptor will recognise an epitope of the dysfunctional P2X7 receptor associated with the ATP-binding site. Accordingly, in some embodiments of the present invention, the antigen-recognition domain of the chimeric antigen receptor, recognises an epitope associated with an adenosine triphosphate (ATP)-binding site of the P2X7 receptor. In some embodiments, the dysfunctional P2X7 receptor has a reduced capacity to bind ATP compared to an ATP-binding capacity of a fully functional P2X7 receptor. In some embodiments the dysfunctional P2X7 receptor cannot bind ATP.

An alteration in one or more amino acids of the P2X7 receptor may include a conformational change in one or more amino acids of the receptor. Therefore, in some embodiments of the invention the antigen recognition domain recognises a dysfunctional P2X7 receptor, wherein the dysfunctional P2X7 receptor has a conformational change that renders the receptor dysfunctional. Specifically, this conformational change may be a change in one or more amino acids of the P2X7 receptor from a trans-conformation to a cis-conformation. In some embodiments, a proline at position 210 of the P2X7 receptor changes from a trans-conformation to a cis-conformation. In this instance, the antigen-recognition domain of the CAR may recognise an epitope that includes proline at amino acid position 210 of the P2X7 receptor. In some embodiments of the first aspect of the present invention, the antigen-recognition domain recognises an epitope that includes one or more amino acids spanning from glycine at amino acid position 200 to cysteine at amino acid position 216 (inclusive) of the dysfunctional P2X7 receptor. In some embodiments of the first aspect of the present invention, the antigen-recognition domain recognises an epitope that includes the proline at position 210 of the dysfunctional P2X7 receptor. In some embodiments of the first aspect of the present invention, the antigen-recognition domain recognises an epitope that includes the proline at position 210 of the dysfunctional P2X7 receptor, and one or more of the amino acid residues spanning from glycine at amino acid position 200 to cysteine at amino acid position 216 (inclusive) of the dysfunctional P2X7 receptor.

Whilst not wanting to be bound by theory, as a result of the conformational change of the proline at position 210 of the P2X7 receptor, the three-dimensional structure of the receptor may be altered. This alteration in the three-dimensional structure may allow the antigen-recognition domain of the CAR to bind to amino acids, or epitopes, previously inaccessible in the native three-dimensional structure of the P2X7 receptor. Therefore, in some embodiments the CAR recognises one or more epitopes of the P2X7 receptor exposed to the antigen-recognition domain as a result of a trans- to cis-conformational change of the proline at position 210 of SEQ ID NO: 3. These epitopes may include one or more of the amino acids at position 200 to 210, or positions 297 to 306, inclusive, of the P2X7 receptor. Accordingly, in some embodiments of the first aspect of the present invention, the antigen-recognition domain recognises an epitope that includes one or more of the amino acids at positions 200 to 210 and/or 297 to 306 of the P2X7 receptor.

As used throughout the specification the term “recognises” relates to the ability of the antigen-recognition domain to associate with a dysfunctional P2X7 receptor, a portion thereof, or an epitope thereof. In some embodiments, the antigen-recognition domain may directly bind to the dysfunctional P2X7 receptor, or an epitope thereof. In other embodiments, the antigen-recognition domain may bind to a processed form of the dysfunctional P2X7 receptor. As used in this context the term “processed form” relates to forms of the P2X7 receptor which have been truncated or digested, typically, as a result of intracellular processing. Consequently, the recognition of the “processed form” of the dysfunctional P2X7 receptor may be as a result of being presented in association with a major histocompatibility complex (MHC).

The antigen-recognition domain can be any suitable domain that can recognise a dysfunctional P2X7 receptor, or epitope thereof. As used throughout the specification the term “antigen-recognition domain” refers to the portion of the CAR that provides the specificity of the CAR for the dysfunctional P2X7 receptor. The antigen-recognition domain, in the context of the present invention, only comprises a portion of the extracellular region (or ectodomain) of the CAR. Suitable antigen-recognition domains, include, but are not limited to, polypeptides having sequence homology to an antigen-binding site of an antibody, or fragment thereof, that bind to a dysfunctional P2X7 receptor. Therefore, in some embodiments of the first aspect of the invention, the antigen-recognition domain includes an amino acid sequence having homology to an antibody, or a portion thereof, that binds to a dysfunctional P2X7 receptor. In some embodiments, a portion of the antigen-recognition domain includes an amino acid sequence having homology to an antibody, or a portion thereof, that binds to the dysfunctional P2X7 receptor. The antibody sequence, to which the antigen-recognition domain has homology with, can be any suitable sequence of an antibody that has an affinity for the P2X7 receptor. For example the sequence can share sequence homology with an antibody originating from one or more of the following species; human, non-human primate, mouse, rat, rabbit, sheep, goat, ferret, canine, chicken, feline, guinea pig, hamster, horse, cow, or pig. The antigen-recognition domain may share sequence homology with the sequence of a monoclonal antibody produced from a hybridoma cell line. When the originating species of the homologous antibody sequence is not human, the antibody is preferably a humanised antibody. The homologous antibody sequence may also be from a non-mammalian animal species such as a cartilaginous fish (e.g. shark IgNAR antibodies—see WO2012/073048). Alternatively, the antigen binding domain may include a modified protein scaffolds that provide functionality similar to shark antibodies, such as i-bodies which have binding moieties based on shark IgNAR antibodies (see WO2005/118629). Additionally, the antigen-recognition domain could be, could be derived from, or could share sequence homology with, any other suitable binding molecule or peptide that can selectively interact with a dysfunctional P2X7 receptor with an affinity sufficient to activate the CAR signalling domain. Methods are known in the art for the identification of antigen-binding proteins such as, inter alia, panning phage display libraries, protein affinity chromatography, co-immunoprecipitation and yeast two-hybrid systems (see Srinivasa Rao, V. et al. Int J Proteomics, 2014; article ID 147648).

In the above context (and as used throughout this specification), the terms “homology” and “homologous” are to be construed in accordance with the definition of “Sequence Homology, Amino Acid” as defined by National Center for Biotechnology Information Medical Subject Headings (NCBI MeSH). As such, the term “homology” and “homologous”, and the like, are to be interpreted as “the degree of similarity between sequences of amino acids”.

In some embodiments, the antigen-recognition domain comprises an amino acid sequence homologous to a single-antibody domain (sdAb) that binds to a dysfunctional P2X7 receptor. In some embodiments, the antigen-recognition domain includes an amino acid sequence homologous to the 3 CDRs from a variable heavy (VH) chain of an antibody, or a variable light (VL) chain of an antibody. In some embodiments, the antigen-recognition domain includes amino acid sequence homology to the amino acid sequence of a multivalent sdAb that binds to a dysfunctional P2X7 receptor. In some embodiments, the multivalent sdAb is a di-valent or tri-valent sdAb.

In some embodiments, the antigen-recognition domain of the CAR includes amino acid sequence homology to the amino acid sequence of a fragment-antigen binding (Fab) portion of an antibody that binds to a dysfunctional P2X7 receptor. As will be understood in the art, a Fab portion of an antibody in composed of one constant region and one variable region of each of the heavy and light chains of an antibody.

In some embodiments of the invention, the antigen-recognition domain includes amino acid sequence homology to the amino acid sequence of a single-chain variable fragment (scFv) that binds to a dysfunctional P2X7 receptor. As would be understood in the art, a scFv is a fusion protein comprising two portions that may share homology with, or may be identical to, the variable-heavy (VH) and variable-light (VL) chains of an antibody, with the two portions connected together with a linker peptide. For example, the scFv may include VH and VL amino acid sequences that are derived from an antibody that recognises a dysfunctional P2X7 receptor.

In the above context it will be appreciated that the term “derived from” is not a reference to the source of the polypeptides per se, but rather refers to the derivation of the amino acid sequence information that constitutes a portion of the antigen-binding region. Consequently, the term “derived from” includes synthetically, artificially or otherwise created polypeptides that share sequence identity to an antibody that binds to the dysfunctional P2X7 receptor.

In some embodiments, the antigen-recognition domain includes an amino acid sequence homologous to the amino acid sequence of a multivalent scFv that binds to a dysfunctional P2X7 receptor. In some embodiments, the multivalent scFv is a di-valent or tri-valent scFv.

In some embodiments, the antigen-recognition domain includes an amino acid sequence set forth in SEQ ID NO: 4, SEQ ID NO: 6, SEQ ID NO: 7 or SEQ ID NO: 8, or functional variants thereof having at least 50%, 60%, 70%, 80%, 90%, 95%, 98% or 99% sequence identity. In some embodiments, the antigen-recognition domain includes an amino acid sequence set forth in SEQ ID NO: 4 or functional variants thereof having at least 50%, 60%, 70%, 80%, 90%, 95%, 98% or 99% sequence identity

In some embodiments, the antigen-recognition domain includes a binding peptide that comprises an amino acid sequence homologous with one or more CDR regions of an antibody that binds to a dysfunctional P2X7 receptor. In some embodiments, the biding peptide includes one or more regions having sequence homology with the CDR1, 2 and 3 domains of the VH and/or VL chain of an antibody that binds to a dysfunctional P2X7 receptor. In some embodiments, the antigen recognition domain includes one or more sequences which are at least 50%, 60%, 70%, 80%, 90% or 94% identical to any one of the CDR regions spanning positions 30 to 35, 50 to 67 or 98 to 108 of the sequences set forth in SEQ ID NOS: 4, 6, 7 or 8. In some embodiments, the antigen-recognition domain includes one or more of the sequences spanning positions 30 to 35, 50 to 67 or 98 to 108 of the sequences set forth in SEQ ID NOS: 4, 6, 7 or 8. The sequences interspacing the CDR regions of the antigen binding peptides set forth in SEQ ID NOS: 4, 6, 7 or 8 can be any suitable sequence that permits the appropriate formation and conformation of the CDR regions. In some embodiments, the antigen-recognition domain includes a sequence 50%, 60%, 70%, 80% or 90%, 95% or 99% identical to one of the sequences set forth in SEQ ID NOS: 4, 6, 7 or 8.

Antibodies directed against dysfunctional P2X7 receptors, from which suitable amino acid sequences may be derived, and methods for producing such antibodies, have been described in the art (for example WO2001/020155, WO2003/020762, WO2008/043145, WO2008/043146, WO2009/033233, WO2011/020155 and WO2011/075789). Methods for generating polyclonal and monoclonal antibodies for specific epitopes (such as those set forth previously) would be known to a person skilled in the art. By way of summary, a desired epitope (such as a segment of the dysfunctional P2X7 receptor including the proline at position 210) is injected into a suitable host animal in the presence of an appropriate immunogenic carrier protein and optionally an adjuvant. Serum is then collected from the immunized animal and the antibody can be isolated based on its antibody class or its antigen specificity. Following assessment of the suitability and specificity of the purified antibody, the antibody can be further processed to isolate antigen-binding fragments, or sequenced to identify the relevant VH and VL domains. Suitable epitopes for the production of antibodies directed against the dysfunctional P2X7 receptor are known in the art (see WO2008/043146, WO2010/000041 and WO2009/033233 as examples).

### Linker Domain

The linker domain connects transmembrane domain and antigen recognition domain. CAR T cells have been formed that function without the inclusion of a linker domain, and therefore, in this context, a linker domain is not considered to be generally essential to the function of all CARs. However, as indicated above, and without wanting to be bound by theory, a linker domain may provide an appropriate molecular length to the ectodomain (extracellular domain) of the CAR to allow recognition of the epitope by the antigen recognition domain, while forming the correct immunological synaptic distance between the effector cell expressing the CAR, and the target cell. Further, the linker domain may provide the appropriate flexibility for the antigen recognition domain to be orientated in the correct manner to recognise its epitope.

The selection of a suitable linker domain can be predicated on (i) reducing binding affinity to Fc Receptors (such as the Fcy and FcRn receptor), which minimizes ‘off-target’ activation of CAR expressing cells and (ii) optimizing the efficacy of the CAR construct by enhancing the flexibility of the antigen binding region, reducing spatial constraints for formation of an immune synapse (e.g. reducing steric hindrance and optimising synaptic distance). However, the means by which the hinge is selected is considered unpredictable in the art and is dependent on the specific antigen, and location of the epitope, targeted by the CAR-expressing effector cell.

As indicated in the Examples, cells expressing CARs directed against dysfunctional P2X7 receptors demonstrated little to no reactivity to the majority of cancer cell lines when the linker domain was 12 amino acids in length. Further, cells expressing CARs directed against dysfunctional P2X7 receptors demonstrated little to no reactivity to the majority of cancer cell lines when the linker domain was 228 amino acids. However, only the linker of 119 amino acids demonstrated broad efficacy against the majority of cell lines when transduced into CD3+ T cells as well as purified sub-populations of CD4+ CD8+ T cells. Typically, CARs target upregulated cell markers that are specific for one, or a select few, types of cancer. As such, broad reactivity to a wide range of cancer cell types is not typically considered when designing the CAR, nor is it typically deemed important. However, the dysfunctional P2X7 receptor is expressed by a wide range of cancer types. Consequently, unlike other CARs, a CAR targeting the dysfunctional P2X7 needs to be optimized to a wide variety of cancer cell types.

Furthermore, in some examples cells expressing CARs having a linker domain of 30 amino acids, and directed against dysfunctional P2X7 receptors, demonstrated comparable reactivity to cells expressing CARs having a linker domain of 228 amino acids, when incubated with a cell lines expressing dysfunctional P2X7 receptors.

Consequently, in some embodiments, the linker domain consists of 12 to 228 amino acids, or 30 to 228 amino acids, or 50 to 200 amino acids, or 70 to 180 amino acids, or 90 to 160 amino acids, or 107 to 131 amino acids, or 110 to 130 amino acids, or 115 to 125 amino acids, or 117 to 121 amino acids. Consequently, in some embodiments, the linker domain consists of between 12 to 228 amino acids, or between, 30 and 228 amino acids, or between 50 to 200 amino acids, or between 70 to 180 amino acids, or between 90 to 160 amino acids, or between 107 to 131 amino acids, or between 110 to 130 amino acids, or between 115 to 125 amino acids, or between 117 to 121 amino acids.

As used throughout this specification in reference to numeric ranges the term “between” is to be understood to be non-inclusive of the bounding number. For example between 1 and 10 refers to the range of 2 to 9, inclusive.

In some embodiments, the linker domain consists of about 119 amino acids. In some embodiments, the linker domain consists of 119 amino acids. In some embodiments, the length of the linker domain is 119 amino acids ±50 amino acids, or ±40 amino acids, or ±30 amino acids, or ±20 amino acids, or ±10 amino acids, or ±5 amino acids, or ±2 amino acids, or ±1 amino acid.

In some embodiments, the linker domain consists of 12 to 227 amino acids. In some embodiments, the linker domain consists of 13 to 227 amino acids. In some embodiments, the linker domain consists of 30 to 228 amino acids. In some embodiments, the linker domain consists of 31 to 227 amino acids.

In some embodiments, the linker domain includes a sequence homologous to a hinge region from an immunoglobulin, or a hinge or extracellular region from a membrane bound molecule involved in the formation of a T cell synapse. For example the linker domain may comprise a region having an amino acid sequence homologous to a hinge region from CD4, CD8, CD3, CD7 or CD28 regions.

In some embodiment, the linker domain includes a sequence homologous to a portion of an immunoglobulin. In some embodiments, the portion is one or more of a CH1 region, a CH2 region, a CH3 region, a CH4 region or a hinge region. In some embodiments, the portion is a CH2 region, a CH3 region or a hinge region of an immunoglobulin. In some embodiments, the portion is a CH2 region or a CH3 region and a hinge region of an immunoglobulin. In some embodiments, the immunoglobulin is selected from the IgG subtype.

In some embodiments, the linker domain is homologous to a portion of the Fc region of IgG1, or a functional variant thereof having at least 50%, 60%, 70%, 80%, 90%, 95%, 98%, 99% or 99.5% A sequence identity. In some embodiments, the linker domain is homologous to the Fc region of IgG2, or a functional variant thereof having at least 50%, 60%, 70%, 80%, 90%, 95%, 98%, 99% or 99.5% sequence identity. In some embodiments, the linker domain is homologous to the Fc region of IgG3 or a functional variant thereof having at least 50%, 60%, 70%, 80%, 90%, 95%, 98%, 99% or 99.5% sequence identity. In some embodiments, the linker domain is homologous to the Fc region of IgG4, or a functional variant thereof having at least 50%, 60%, 70%, 80%, 90%, 95%, 98%, 99% or 99.5% sequence identity. In some embodiments, the linker domain includes a sequence having homology to a portion of more than one of IgG1, IgG2, IgG3 or IgG4 Fc regions, for example the IgG1 hinge region and the CH2 or CH3 regions of IgG4.

In some embodiments, the linker domain includes all, or part of, an immunoglobulin hinge region. As would be understood in the art, the specific region that forms the hinge region of an immunoglobulin varies for different isotypes. For example, IgA, IgD and IgG isotype immunoglobulins have a hinge region between the CH1 and CH2 regions, while the function of the hinge region is provided by the CH2 region in IgE and IgM isotype immunoglobulins.

A non-exhaustive list of sequences which may be incorporated into the linker domain is provided in Table 2, below. In some embodiments, the linker domain of the present invention may include any one or more of the components provided in Table 2. In some embodiments, the linker domain may comprise one or more of the linkers provided in Table 2. Further, the linker domain may be an artificially synthesized sequences such poly-Glycine sequences or repeats of GGGGS (Gly₄Ser) sequences (for example a (Gly₄Ser)₃).

TABLE 2
Possible linker domain components

| Name | length | Sequence | SEQ ID |
|---|---|---|---|
| IgG1 | 15 | EPKSCDKTHTCPPCP | 9 |
| hinge | | | |
| IgG2 | 12 | ERKCCVECPPCP | 10 |
| hinge | | | |
| IgG3 | 62 | LKTPLGDTTHTCPRCPEPKSCDTPPPCPRCPEPK | 11 |
| hinge | | SCDTPPPCPRCPEPKSCDTPPPCPRCP | |
| IgG4 | 12 | ESKYGPPCPSCP | 12 |
| hinge | | | |
| IgG4 | 12 | ESKYGPPCPPCP | 13 |
| hinge | | | |
| (mutated) | | | |
| IgA1 | 21 | PVPSTPPTPSPSTPPTPSPSC | 14 |
| hinge | | | |
| IgD hinge | 58 | ESPKAQASSVPTAQPQAEGSLAKATTAPATTRNT | 15 |
| | | GRGGEEKKKEKEKEEQEERETKTP | |
| IgM | 112 | IAELPPKVSVFVPPRDGFFGNPRKSKLICQATGFS | 16 |
| common | | PRQIQVSWLREGKQVGSGVTTDQVQAEAKESGP | |
| heavy | | TTYKVTSTLTIKESDWLGQSMFTCRVDHRGLTFQ | |
| (CH) 2 | | QNASSMCVPD | |
| IgE CH2 | 99 | PTVKILQSSCDGGGHFPPTIQLLCLVSGYTPGTINI | 17 |
| | | TWLEDGQVMDVDLSTASTTQEGELASTQSELTL | |
| | | SQKHWLSDRTYTCQVTYQGHTFEDSTKKCA | |
| CD28 | 15 | KHLCPSPLFPGPSKP | 18 |
| Hinge | | | |
| CD8a | 48 | AKPTTTPAPRPPTPAPTIASQPLSLRPEACRPAA | 19 |
| Hinge (1) | | GGAVHTRGLDFACD | |
| CD8a | 45 | TTTPAPRPPTPAPTIASQPLSLRPEACRPAAGGA | 20 |
| Hinge (2) | | VHTRGLDFACD | |
| CD8b | 38 | SVVDFLPTTAQPTKKSTLKKRVCRLPRP | 21 |
| Hinge (1) | | ETQKGPLCSP | |
| CD8b | 36 | VDFLPTTAQPTKKSTLKKRVCRLPRP | 22 |
| Hinge (2) | | ETQKGPLCSP | |
| CD7 | 36 | APPRASALPAPPTGSALPDPQTASALPDPPAASA | 23 |
| | | LP | |
| CD4 | 23 | DSGQVLLESNIKVLPTWSTPVQP | 24 |
| Hinge | | | |
| PolyG/S | 5+ | (GGGGS)ₙ | 25 |

In some embodiments, the linker domain includes a sequence homologous to any one or more of the sequences selected from SEQ ID NOs: 9 to 25 and 30 to 37, or a functional variant, or portion thereof, having at least 50%, 60%, 70%, 80%, 90%, 95%, 98%, 99% or 99.5% sequence identity.

In some embodiments, the linker domain includes a sequence homologous to an immunoglobulin CH3 domain, an immunoglobulin CH2 domain or both a CH2 and CH3 domain. In some embodiments, the linker domain includes a sequence homologous to an immunoglobulin hinge region and one or more of a CH3 domain or a CH2 domain. The immunoglobulin sequences can include one or more amino acid modifications, for example, 1, 2, 3, 4 or 5 substitutions, deletions, insertions or additions, e.g., substitutions that reduce Fc Receptor (FcR) or Fc Receptor neonatal (FcRn) binding.

The term “substitution” refers to replacement of an amino acid at a particular position in a parent peptide or protein sequence with another amino acid. A substitution can be made to change an amino acid in the resulting protein in a non-conservative manner (e.g., by changing the amino acid belonging to a grouping of amino acids having a particular size or characteristic to an amino acid belonging to another grouping; e.g. substituting a hydrophilic amino acid with a hydrophobic amino acid) or in a conservative manner (e.g., by changing the amino acid belonging to a grouping of amino acids having a particular size or characteristic to an amino acid belonging to the same grouping; e.g. substituting a hydrophilic amino acid with a hydrophilic amino acid). Such a conservative change generally leads to a reduction in conformational and functional changes in the modified peptide/protein. The following are examples of various groupings of amino acids: 1) Amino acids with nonpolar R groups: Alanine, Valine, Leucine, Isoleucine, Proline, Phenylalanine, Tryptophan, Methionine; 2) Amino acids with uncharged polar R groups: Glycine, Serine, Threonine, Cysteine, Tyrosine, Asparagine, Glutamine; 3) Amino acids with charged polar R groups (negatively charged at pH 6.0): Aspartic acid, Glutamic acid; 4) Basic amino acids (positively charged at pH 6.0): Lysine, Arginine, Histidine (at pH 6.0). Another grouping may be those amino acids with phenyl groups: Phenylalanine, Tryptophan, and Tyrosine.

A person skilled in the art will recognise that any amino acid can be substituted with a chemically (functionally) similar amino acid and retain function of the polypeptide. Such conservative amino acid substitutions are well known in the art. The following groups in Table 3 each contain amino acids that are conservative substitutions for one another.

TABLE 3
Exemplary amino acid conservative substitutions

| Original | |
|---|---|
| Residue | Exemplary Substitutions |
| Ala (A) | Val (V), Leu (L), Ile (I), Gly (G) |
| Arg (R) | Lys (K) |
| Asn (N) | Gln (Q), His (H) |
| Asp (D) | Glu (E) |
| Cys (C) | Ser (S) |
| Gln (Q) | Asn (N), His (H) |
| Glu (E) | Asp (D) |
| Gly (G) | Pro (P), Ala (A) |
| His (H) | Asn (N), Gln (Q) |
| Ile (I) | Leu (L), Val (V), Ala (A) |
| Leu (L) | Ile (I), Val (V), Met (M), Ala |
| | (A), Phe (F) |
| Lys (K) | Arg (R) |
| Met (M) | Leu (L), Phe (F) |
| Phe (F) | Leu (L), Val (V), Alal (A) |
| Pro (P) | Gly (G) |
| Ser (S) | Thr (T) |
| Thr (T) | Ser (S) |
| Trp (W) | Tyr (Y) |
| Tyr (Y) | Trp (W), Phe (F) |
| Val (V) | Ile (I), Leu (L), Met (M), |
| | Phe (F), Ala (A) |

The term “insertion” refers to addition of amino acids within the interior of the sequence. “Addition” refers to addition of amino acids to the terminal ends of the sequence. “Deletion” refers to removal of amino acids from the sequence.

In some embodiments, the chimeric antigen receptor includes a linker domain that includes an amino acid sequence homologous to an immunoglobulin hinge region of IgG, IgD, IgA, or a constant heavy 2 (CH2) region of IgM or IgE, or a function variant thereof having at least 50%, 60%, 70%, 80%, 90%, 93%, ₉₆% _{or 99}% sequence identity.

In some embodiments, the linker domain of the chimeric antigen receptor includes an amino acid sequence homologous to a hinge region from an IgG isotype immunoglobulin, or a functional variant thereof having at least 50%, 60%, 70%, 80%, 90%, 93%, 96% or 98% sequence identity. In some embodiments, the linker domain includes an amino acid sequence homologous to an IgG1, IgG2, IgG3, or IgG4 hinge region, or a functional variant having at least 50%, 66%, 73%, 75%, 80%, 83%, 86%, 91%, 93%, 96% or 98% sequence identity. In some embodiments, the linker domain includes an amino acid sequence homologous to an IgG1, IgG2, IgG3, or IgG4 hinge region includes one or more amino acid residues substituted with an amino acid residue different from that present in the unmodified hinge domain. In some embodiments, the linker domain includes an amino acid sequence homologous to the hinge region of the IgG1, IgG2 or IgG4, or a functional variant thereof having at least 50%, 66%, 73%, 75%, 80%, 83%, 86%, 91% or 93% sequence identity.

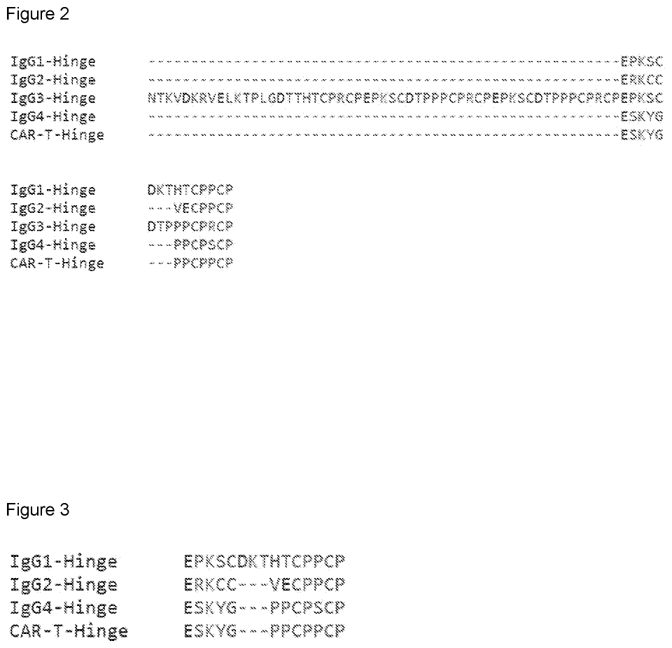

An alignment of the IgG subtype hinge regions and the IgG4 (mutated) hinge region (as used in an embodiment of this invention—“CAR-T-hinge”) is provided in FIG. 2. Further an alignment of IgG1, IgG2 and IgG4 hinges regions and the IgG4 (mutated hinge region) is provided in FIG. 3. As can be seen, there is a high degree of homology between the IgG1, IgG2 and IgG4 hinge regions, and a portion of IgG3.

In some embodiments, the sequence homologous to a hinge region from an IgG isotype immunoglobulin and includes a CXXC motif, wherein “C” is a Cysteine and “X” is any amino acid. In some embodiments the CXXC motif is selected from the group consisting of CPPC, CPRC or CPSC. In a preferred embodiment, the CXXC motif is CPPC. In some embodiments, the sequence homologous to the hinge region is modified to include a CPPC motif.

In some embodiments, the linker domain of the chimeric antigen receptor includes one or more amino acid sequences homologous to a CH region of an immunoglobulin. In some embodiments, the amino acid sequence homologous to a CH region is homologous to one or more of a CH1 region, a CH2 region, a CH3 region or a CH4 region of an immunoglobulin, or has 50%, 60%, 70%, 80%, 90%, 95%, 98% or 99% sequence identity with said CH regions.

In some embodiments, the linker domain of the chimeric antigen receptor includes one or more amino acid sequences homologous to one or more of a CH2 region or a CH3 region of an IgG isotype immunoglobulin or has 50%, 60%, 70%, 80%, 90%, 95%, 98% or 99% A sequence identity with said CH2 or CH3 regions.

In some embodiments, the linker domain of the chimeric antigen receptor includes one or more immunoglobulin hinge region(s) and/or one or more CH region(s) of an immunoglobulin. In some embodiments, the linker domain of the chimeric antigen receptor consists of an immunoglobulin hinge region and a CH region, preferably a CH2 region or a CH3 region. In some embodiments the CH2 and/or CH3 regions are from an IgG isotype immunoglobulin. In some embodiments the CH2 and/or CH3 regions are from the IgG4 subclass of IgG antibodies.

In some embodiments, the linker domain of the chimeric antigen receptor includes an amino acid sequence according to SEQ ID Nos: 9 to 17, or a functional variant or a functional part thereof having at least 50%, 60%, 70%, 80%, 90%, 95%, 98% or 99% sequence identity, preferably the chimeric antigen receptor includes an amino acid sequence according to SEQ ID Nos: 9 to 13, or a functional variant or a functional part thereof having at least 50%, 60%, 70%, 80%, 90%, 95%, 98% or 99% sequence identity. In some embodiments, the linker domain includes, or consists of, an amino acid sequence according to SEQ ID NO: 39, or a functional variant thereof having at least 50%, 66%, 73%, 75%, 80%, 85%, 90%, 95%, 96%, 97%, 98% or 99% sequence identity.

The hinge region, CH2 and CH3 region of immunoglobulins, in particular IgG isotype antibodies, may be bound by Fc receptors such as Fc gamma receptors and Fc neonatal receptors. Binding of the linker domain of a chimeric antigen receptor can reduce the efficacy of the receptor and can lead to off-target killing. Therefore, in some embodiments, the linker domain is designed such that it has a reduced, or no, capacity to bind with an Fc receptor. In some embodiments, the linker domain is homologous to an immunoglobulin with a reduced capacity to bind with an Fc receptor compared to other immunoglobulin isotypes. In some embodiments, the linker domain of the chimeric antigen receptor does not comprise an amino acid sequence in the linker domain that substantially binds with an Fc receptor.

The ability for Fc receptors to bind with different IgG isotypes is presented in Table 4 below.

TABLE 4
Fc Receptor binding to IgG subtypes3

| | IgG1 | IgG2 | IgG3 | IgG4 |
|---|---|---|---|---|
| FcγRIa (CD64) | +++ | − | ++++ | ++ |
| FcγRIIaa (CD32) | +++ | ++ | ++++ | ++ |
| FcγRIIba (CD32) | + | − | ++ | + |
| FcγRIIIba (CD16a) | +++ | − | ++++ | − |
| FcRn | +++ | +++ | +++ | +++ |

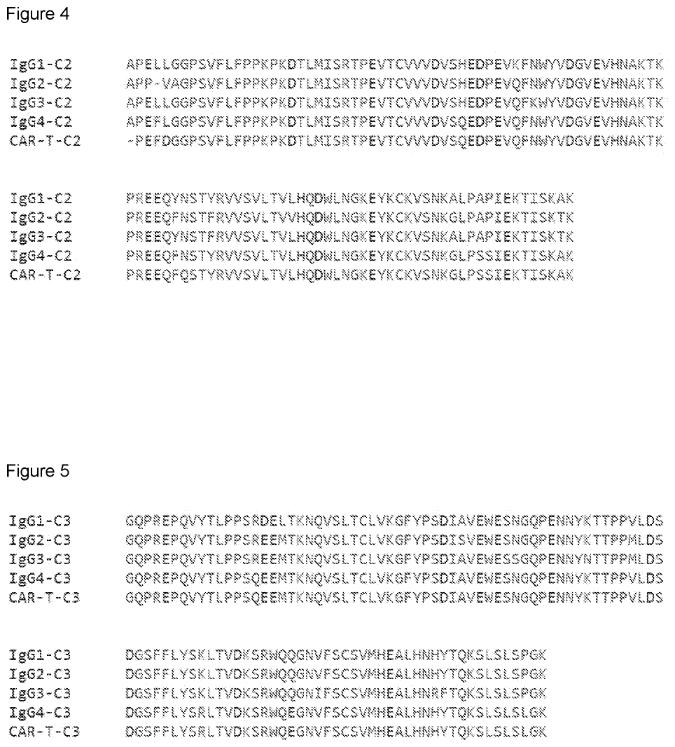

In some embodiments, where the linker domain includes a portion homologous to the Fc region of an immunoglobulin, the portion maybe modified to reduce binding to the Fc receptor. Methods are known in the art for modifying Fc regions to reduce binding by Fc Receptors. Fc gamma receptor primarily binds to the lower hinge region and the n-terminal of the CH2 region of immunoglobulin regions, while the neonatal Fc receptor primarily binds to amino acids at the c terminus of the CH2 region and the N terminus of the CH3 region. A guide to the binding of Fc receptors to IgG antibodies can be found in Chapter 7 of “Antibody Fc : Linking Adaptive and Innate Immunity” Ackerman and Nimmerjahn, Elsevier Science & Technology 2014. Therefore, modifications in these areas may alter the binding of Fc receptors to linker domains having homology with the Fc portion of immunoglobulins. A non-exhaustive exemplary list of mutations to Human IgG1, which have been shown to reduce Fc-gamma receptor and FcRn binding include: E116P, L117V, L118A, G119 deleted, P121A, S122A, I136A, S137A, R138A, T139A, E141A, D148A, 5150A, 5150A, E152A, D153A, E155A, N159A, D163A, H168A, N169A, K171A, K173A, R175A, E176A, Q178A, Y179F, N180A, S181A, R184A, V188A, T190A, L192A, Q194A, D195A, N198A, K200A, K205A, K209A, A210Q, A210S, A210G, P212A, P214A, E216A, K217A, S220A, K221A, A222T, K243A, Q245A, H251A, D259A, A261Q, E263A, E265A, V286A, S288A, K297A, 5307A, E313A, H316A, N317A, H318A, Y319A (numbering corresponds to the sequence set forth in Uniprot reference number P01857-1 and SEQ ID NO: 26). Comparisons of the CH2 and CH3 regions of the four IgG subtypes, and the CH2 and CH3 regions used in examples provided herein, are provided in FIGS. 4 and 5.

### Transmembrane and Intracellular Domains

The transmembrane domain of a CAR bridges the extracellular portion (ectodomain) to the intracellular portion (endodomain) with its role being primarily structural. As such, the transmembrane domain can consist of any sequence that can anchor and span the lipid bilayer of a cell. However, the nature of the transmembrane domain can influence its localisation and expression.

In a preferred embodiment, the transmembrane domain has homology to a sequence of a molecule involved in T cell synapse formation, or T cell signal induction. In some embodiments, the chimeric antigen receptor of the present invention includes a transmembrane domain which includes a sequence homologous to all, or part of, the transmembrane domain of CD3, CD4, CD8 or CD28. In some embodiments, the transmembrane domain includes a sequence homologous to all, or part of, the transmembrane domain of CD8 or CD28. In some embodiments, the transmembrane domain includes a sequence homologous to all, or part of, the transmembrane domain of CD28.

In addition to the antigen recognition domain, the linker domain and the transmembrane domain, the chimeric antigen receptor of the present invention includes an intracellular (endo) domain which includes a signalling portion (a signalling domain).

The intracellular signalling domain of the chimeric antigen receptor can be any suitable domain that is capable of inducing, or participating in the induction of, an intracellular signalling cascade upon activation of the CAR as a result of recognition of an antigen by the antigen-recognition domain. The signalling domain of a CAR will be specifically chosen depending on the cellular outcome desired following activation of the CAR. Whilst there are many possible signalling domains, when used in immunotherapy and cancer therapy the signalling domains can be grouped into two general categories based on the receptor from which they are derived, namely activation receptors and co-stimulatory receptors (see further details below). Therefore, in some embodiments, the signalling domain includes a portion derived from an activation receptor. In some embodiments, the signalling domain includes a portion derived from a co-stimulatory receptor

As used throughout the specification the term “portion”, when used with respect to an activation receptor or co-stimulatory receptor, relates to any segment of the receptor that includes a sequence responsible for, or involved in, the initiation/induction of an intracellular signalling cascade following interaction of the receptor with its cognate antigen or ligand. An example of the initiation/induction of an intracellular signalling cascade for the T cell receptor (TCR) via CD3 is outlined below.

Whilst not wishing to be bound by theory, the extracellular portion of the TCR largely comprises heterodimers of either the clonotypic TCRα and TCRβ chains (the TCRα/β receptor) or the TCRγ and TCRδ chains (the TCRγδ receptor). These TCR heterodimers generally lack inherent signalling transduction capabilities and therefore they are non-covalently associated with multiple signal transducing subunits of CD3 (primarily CD3-zeta, -gamma, -delta, and -epsilon). Each of the gamma, delta, and epsilon chains of CD3 has an intracellular (cytoplasmic) portion that includes a single Immune-receptor-Tyrosine-based-Activation-Motif (ITAM), whilst the CD3-zeta chain includes three tandem ITAMs. Upon engagement of the TCR by its cognate antigen in the presence of MHC, and the association of a requisite co-receptor such as CD4 or CD8, signalling is initiated which results in a tyrosine kinase (namely Lck) phosphorylating the two tyrosine residues within the intracellular ITAM(s) of the CD3 chains. Subsequently, a second tyrosine kinase (ZAP-70—itself activated by Lck phosphorylation) is recruited to biphosphorylate the ITAMs. As a result, several downstream target proteins are activated which eventually leads to intracellular conformational changes, calcium mobilisation, and actin cytoskeleton re-arrangement that when combined ultimately lead to activation of transcription factors and induction of a T cell immune response.

As used throughout the specification the term “activation receptor” relates to receptors, or co-receptors that form a component of, or are involved in the formation of, the T cell receptor (TCR) complex, or receptors involved in the specific activation of immune cells as a result of recognition of an antigenic or other immunogenic stimuli.

Non-limiting examples of such activation receptors include components of the T cell receptor-CD3 complex (CD3-zeta, -gamma, -delta, and -epsilon), the CD4 co-receptor, the CD8 co-receptor, Fc receptors or Natural Killer (NK) cell associated activation receptors such a LY-49 (KLRA1), natural cytotoxicity receptors (NCR, preferably NKp46, NKp44, NKp30 or NKG2 or the CD94/NKG2 heterodimer). Consequently, in some embodiments of the first aspect of the present invention, the signalling domain includes a portion derived from any one or more of a member of the CD3 co-receptor complex (preferably the CD3-Zeta (ζ) chain), the CD4 co-receptor, the CD8 co-receptor, a Fc Receptor (FcR) (preferably the FcεRI or FcγRI) or NK associated receptors such a LY-49.

The specific intracellular signal transduction portion of each of the CD3 chains are known in the art. By way of example, the intracellular cytoplasmic region of the CD3ζ chain spans from amino acid 52 to amino acid 164 of the sequence set forth in SEQ ID NO: 42, with the three ITAM regions spanning amino acids 61 to 89, 100 to 128 and 131 to 159 of SEQ ID NO: 42. Furthermore, the intracellular portion of the CD3ε chain spans amino acids 153 to 207 of the sequence set forth in SEQ ID NO: 43, with the single ITAM region spanning amino acids 178 to 205 of SEQ ID NO: 43. The intracellular portion of CD3γ chain spans amino acids 138 to 182 of the sequence set forth in SEQ ID NO: 44 with the single ITAM region spanning amino acids amino acids 149 to 177 of SEQ ID NO: 44. The intracellular portion of CD3δ spans amino acids 127 to 171 of the sequence set forth in SEQ ID NO: 45 with the single ITAM region spanning amino acids 138 to 166 of SEQ ID NO: 45.

In some embodiments of the present invention, the signalling domain includes a portion derived from, or having sequence homology to, CD3 (preferably the CD3-ζ chain or a portion thereof). In some embodiments, the signalling domain includes a signal homologous to all, or part of, the intracellular domain of CD3 zeta (CD3-ζ). In some embodiments, the portion of the CD3-ζ co-receptor complex includes the amino acid sequence set forth in SEQ ID NO: 46, or a functional variant thereof having at least 50%, 60%, 70%, 80%, 90%, 95%, 98%, 99% or 99.5% sequence identity.

Alternative signalling domains include intracellular portions of the Fc receptors, which are known in the art. For example, the intracellular portions of the FcεR1 span amino acids 1 to 59, 118 to 130 and 201 to 244 of the sequence set forth in SEQ ID NO: 47. Furthermore, the intracellular portion of FcγRI spans the amino acids 314 to 374 of the sequence set forth in SEQ ID NO: 48.

Various combinations of portions of activation receptors can be utilized to form the transmembrane (TM) and intracellular (IC) portions of the CAR for example the CD3ζ TM and CD3ζ IC (Landmeier S. et al. Cancer Res. 2007; 67:8335-43; Guest RD. et al., J Immunother. 2005, 28:203-11; Hombach A A. et al. J Immunol. 2007; 178: 4650-7), the CD4 TM and CD3ζ IC (James S E. et al. J Immunol. 2008; 180:7028-38), the CD8 TM and CD3ζ IC (Patel S D. et al. Gene Ther. 1999; 6: 412-9), and the FcεRIγ TM and the FcεRIγ IC (Haynes N M. et al. J Immunol. 2001; 166: 182-7; Annenkov A E. et al. J Immunol. 1998; 161: 6604-13).

As used throughout the specification the term “co-stimulatory receptor” relates to receptors or co-receptors that assist in the activation of an immune cell upon antigen specific inducement of an activation receptor. As will be understood, co-stimulatory receptors do not require the presence of antigen and are not antigen specific, but are typically one of two signals, the other being an activation signal, which is required for the induction of an immune cellular response. In the context of an immune response a co-stimulation receptor is typically activated by the presence of its expressed ligand on the surface of an antigen-presenting cell (APC) such as a dendritic cell or macrophage. With specific regard to T cells, co-stimulation is necessary to lead to cellular activation, proliferation, differentiation and survival (all of which are generally referred to under the umbrella of T cell activation), whilst presentation of an antigen to a T cell in the absence of co-stimulation can lead to anergy, clonal deletion and/or the development of antigen specific tolerance. Importantly, co-stimulatory molecules can inform the T cell response to a simultaneously encountered antigen. Generally, an antigen encountered in the context of a ‘positive’ co-stimulatory molecule will lead to activation of the T cell and a cellular immune response aimed at eliminating cells expressing that antigen. Whilst an antigen encountered in the context of a ‘negative’ co-receptor will lead to an induced state of tolerance to the co-encountered antigen.

Non-limiting examples of T cell co-stimulatory receptors include CD27, CD28, CD30, CD40, DAP10, OX40, 4-1BB (CD137), ICOS. Specifically, CD27, CD28, CD30, CD40, DAP10, OX40, 4-1BB (CD137), and ICOS all represent ‘positive’ co-stimulatory molecules that enhance activation of a T cell response. Accordingly, in some embodiments of the first aspect of the present invention, the signalling domain includes a portion derived from any one or more of CD27, CD28, CD30, CD40, DAP10, OX40, 4-1BB (CD137) and ICOS.

In some embodiments of the present invention, the signalling domain includes a portion derived from the CD28, OX40 or 4-1BB co-stimulatory receptors. In some embodiments, the signalling domain includes a portion of 4-1BB. In some embodiments, the portion of the 4-1BB co-stimulatory receptor includes the amino acid sequence set forth in SEQ ID NO: 49, or a functional variant or portion thereof having at least 50%, 60%, 70%, 80%, 90%, 95%, 98%, 99% or 99.5% A sequence identity.

Various combinations of portions of co-stimulatory receptors can be utilized to form the transmembrane (TM) and intracellular (IC) portions of the CAR. For example the CD8 TM and DAP10 IC or CD8 TM and 4-1BB IC (Marin V. et al. Exp Hematol. 2007; 35: 1388-97), the CD28 TM and the CD28 IC (Wilkie S. et al. J Immunol. 2008;180: 4901-9; Maher J. et al. Nat Biotechnol. 2002; 20: 70-5), and the CD8 TM and the CD28 IC (Marin V. et al. Exp Hematol. 2007; 35: 1388-97).

Sequence information for the above-referenced activation and co-stimulatory receptors is readily accessible in a variety of databases. For example, embodiments of human amino acid, gene and mRNA sequences for these receptors are provided in Table 5.

TABLE 5
Summary of Activation and Co-stimulation
Receptor Sequence Information

| | Uniprot | NCBI Gene | GeneBank mRNA |
|---|---|---|---|
| Receptor Name | Ref No. | ID No. | Ref No. |
| CD3-zeta | P20963 | 919 | GI: 166362721 |
| CD3-gamma | P09693 | 917 | GI: 166362738 |
| CD3-delta | P04234 | 915 | GI: 98985799 |
| CD3-epsilon | P07766 | 916 | GI: 166362733 |
| CD4 | P0173 | 920 | GI: 303522473 |
| CD8 alpha | P01732 | 925 | GI: 225007534 |
| CD8 beta | P01966 | 926 | GI: 296010927 |
| FcγRI | P12314 | 2209 | GI: 31331 |
| FcεR1 | Q01362 | 2206 | GI: 219881 |
| Ly-49 (KLRAI) | Q7Z556 | 10748 | GI: 33114184 |
| NKp46 | O76036 | 9437 | GI: 3647268 |
| NKp44 | O95944 | 9436 | GI: 4493701 |
| NKp30 | O14931 | 259197 | GI: 5823969 |
| CD94 | Q13241 | 3824 | GI: 1098616 |
| CD27 | P26842 | 939 | GI: 180084 |
| CD28 | P10747 | 940 | GI: 338444 |
| CD30 | P28908 | 943 | GI: 180095 |
| CD40 | P25942 | 958 | GI: 29850 |
| DAP10 | Q9UBK5 | 10870 | GI: 5738198 |
| OX40 | P43489 | 7293 | GI: 472957 |
| 4-1BB (TNR9) | Q07011 | 3604 | GI: 571320 |
| ICOS | Q9Y6W8 | 29851 | GI: 9968295 |
| CTLA-4 | P16410 | 1493 | GI: 291928 |
| PD-1 | Q15116 | 5133 | GI: 2149002 |

Whilst Table 5 is provided with reference to human activation and co-stimulatory receptors, it would be understood by a person skilled in the art that homologous and orthologous versions of each receptor are present in the majority of mammalian and vertebrate species. Therefore, the above-referenced sequences are only provided as non-limiting examples of receptor sequences that may be included in a CAR of the first aspect of the present invention and homologous and orthologous sequences from any desired species may be used to generate a CAR that is suitable for the given species.

In some embodiments of the invention, the transmembrane domain and a portion of the signalling domain share homology with the same molecule. For example a portion of CD3 including the transmembrane domain and a signalling domain may be utilised. In some embodiments the transmembrane domain includes, or consists of, a sequence homologous to all or a portion of the transmembrane domain of CD28 and the signalling domain includes, or consists of, all or a portion of the intracellular domain of CD28.

In some embodiments of the present invention, the signalling domain includes a portion derived from an activation receptor and a portion derived from a co-stimulatory receptor. Whilst not wishing to be bound by theory, in this context the recognition of an antigen by the antigen-recognition domain of the CAR will simultaneously induce both an intracellular activation signal and an intracellular co-stimulatory signal. Consequently, this will simulate the presentation of an antigen by an APC expressing co-stimulatory ligand. Alternatively, the CAR could have a signalling domain that includes a portion derived from either an activation receptor or a co-stimulatory receptor. In this alternative form, the CAR will only induce either an activating intracellular signalling cascade or a co-stimulatory intracellular signalling cascade.

In some embodiments of the invention the signalling domain includes, or consists of, all or a portion of the intracellular domain of 4-1BB and CD3-ζ chain.

In some embodiments, the CAR will have a signalling domain that includes a portion derived from a single activation receptor and portions derived from multiple co-stimulatory receptors. In some embodiments, the CAR will have a signalling domain that includes portions derived from multiple activation receptors and a portion derived from a single co-stimulatory receptor. In some embodiments, the CAR will have a signalling domain that includes portions derived from multiple activation receptors and portions derived from multiple co-stimulatory receptors. In some embodiments, the CAR will have a signalling domain that includes a portion derived from a single activation receptor and portions derived from two co-stimulatory receptors. In some embodiments, the CAR will have a signalling domain that includes a portion derived from a single activation receptor and portions derived from three co-stimulatory receptors. In some embodiments, the CAR will have a signalling domain that includes portions derived from two activation receptors, and a portion derived from one co-stimulatory receptor. In some embodiments, the CAR will have a signalling domain that includes portions derived from two activation receptors and portions derived from two co-stimulatory receptors. As will be understood there are further variations of the number of activation receptors and co-stimulatory receptors from which the signalling domain can be derived from, and the above examples are not considered to be limiting on the possible combinations included herein.

In some embodiments of the invention, the transmembrane domain and a portion of the signalling domain share homology with different molecules. In some embodiments, the transmembrane domain includes, or consists of, a sequence homologous to all or a portion of the transmembrane domain of CD28 and the signalling domain includes, or consists of, all or a portion of the intracellular domain of 4-1 BB and CD3-ζ chain.

### Chimeric Antigen Receptor

In an embodiment of the present invention, the chimeric antigen receptor includes an antigen-recognition domain recognising a dysfunctional P2X7 receptor, a linker domain including a sequence homologous to the hinge and CH3 region of the IgG4 heavy chain, a transmembrane domain including a sequence homologous to the transmembrane portion of CD28 and an activation domain including the intracellular portion of the CD3 zeta chain and the cytoplasmic region of 4-1 BB, or functional portion or equivalent thereof having 50%, 60%, 70%, 80%, 90%, 95%, 98%, 99%, 99.5% or 99.9% sequence identity to any one of homologues portions.

In some embodiments of invention, the chimeric antigen receptor includes, or consists of, the amino acid sequence set forth in SEQ ID NO: 50, or SEQ ID NO: 51, or a functional variant of SEQ ID NO: 50 or SEQ ID NO: 51. In some embodiments, the functional variant includes an amino acid sequence which is at least 80% identical to SEQ ID NO: 50 or SEQ ID NO: 51. In the context of the present invention, a “functional variant” may include any amino acid sequence provided it maintains the function of any one of the above sequences. As such, the functional variant may, for example, have one or more amino acid insertions, deletions or substitutions relative to one of SEQ ID NO: 50 or SEQ ID NO: 51; a mutant form or allelic variant; an ortholog; a homeologue; an analog of one of SEQ ID NO: 50 or SEQ ID NO: 51; and the like, provided the functional variant maintains the function of any one of SEQ ID NO: 50 or SEQ ID NO: 51.

For example with respect to SEQ ID NO: 50 or SEQ ID NO: 51 the preferred function of a chimeric antigen receptor is to recognise a dysfunctional P2X7 receptor without significant recognition of the functional P2X7 receptor, and induce an intracellular signal which results in the activation of a T cell expressing the CAR. As would be understood by a person skilled in the art, variation to portions of the amino acid sequence of the chimeric antigen receptor set forth in SEQ ID NO: 50 or SEQ ID NO: 51 may be made without significant alteration of the recognition of the dysfunctional P2X7 receptor and/or activation of a T cell expressing the CAR. Such variations may include, but are not limited to, variations in the hinge region of the chimeric antigen receptor, variations in the transmembrane domain, and variations in the portions of the activation receptors and/or co-stimulatory receptors that comprise the intracellular domain of the chimeric antigen receptor.

In some embodiments, a functional variant may comprise at least 85% amino acid sequence identity, at least 90% amino acid sequence identity, at least 91% amino acid sequence identity, at least 92% amino acid sequence identity, at least 93% amino acid sequence identity, at least 94% amino acid sequence identity, at least 95% amino acid sequence identity, at least 96% amino acid sequence identity, at least 97% amino acid sequence identity, at least 98% amino acid sequence identity, at least 99% amino acid sequence identity, or at least 99.1%, 99.2%, 99.3%, 99.4%, 99.5%, 99.6%, 99.7%, 99.8%, or 99.9% amino acid sequence identity to any one of SEQ ID NO: 50 or SEQ ID NO: 51.

When comparing amino acid sequences, the sequences should be compared over a comparison window which is determined by the length of the polypeptide. For example, a comparison window of at least 20 amino acid residues, at least 50 amino acid residues, at least 75 amino acid residues, at least 100 amino acid residues, at least 200 amino acid residues, at least 300 amino acid residues, at least 400 amino acid residues, at least 500 amino acid residues, at least 600 amino acid residues, or over the full length of any one of the sequences listed in Table 1 is envisaged. The comparison window may comprise additions or deletions of about 20%, about 18%, about 16%, about 14% about 12%, about 9%, about 8%, about 6%, about 4% or about 2% or less as compared to the reference sequence (which does not comprise additions or deletions) for optimal alignment of the two sequences. Optimal alignment of sequences for aligning a comparison window may be conducted by computerised implementations of algorithms such as the BLAST family of programs as, for example, disclosed by Altschul et al., Nucl. Acids Res. 1997; 25: 3389-3402. Global alignment programs may also be used to align similar sequences of roughly equal size. Examples of global alignment programs include NEEDLE (available at www.ebi.ac.uk/Tools/psa/emboss_needle/) which is part of the EMBOSS package (Rice P et al., Trends Genet., 2000; 16: 276-277), and the GGSEARCH program (available at fasta.bioch.virginia.edu/fasta_www2/fasta_www.cgi?rm=compare&pgm=gnw) which is part of the FASTA package (Pearson W and Lipman D, 1988, Proc. Natl. Acad. Sci. USA, 85: 2444-2448). Both of these programs are based on the Needleman-Wunsch algorithm which is used to find the optimum alignment (including gaps) of two sequences along their entire length. A detailed discussion of sequence analysis can also be found in Unit 19.3 of Ausubel et al (“Current Protocols in Molecular Biology” John Wiley & Sons Inc, 1994-1998, Chapter 15, 1998).

### Nucleic Acid Constructs and Genetic Modification of Cells

The CAR described herein can be produced by any means known in the art, though preferably it is produced using recombinant DNA techniques. Nucleic acids encoding the several regions of the chimeric receptor can be prepared and assembled into a complete coding sequence by standard techniques of molecular cloning known in the art (genomic library screening, PCR, primer-assisted ligation, site-directed mutagenesis, etc.) as is convenient. The resulting coding region is preferably inserted into an expression vector and used to transform a suitable expression host cell line, preferably a T lymphocyte cell line, and most preferably an autologous T lymphocyte cell line.

As such, the present invention further provides a nucleic acid molecule, or a nucleic acid construct, including a nucleic acid molecule encoding the chimeric antigen receptor described above. In some embodiments, the nucleic acid molecule is a non-naturally occurring and/or synthetic nucleic acid molecule.

In some embodiments, the nucleic acid molecule includes a nucleotide sequence which encodes the amino acid sequence set forth in SEQ ID NO: 50 or SEQ ID NO: 51. In some embodiments, the functional variant includes an amino acid sequence which is at least 80% identical to SEQ ID NO: 50 or SEQ ID NO: 51.

The nucleic acid molecule may comprise any polyribonucleotide or polydeoxyribonucleotide, which may be unmodified, or modified, RNA or DNA. For example, the nucleic acid molecule may include single- and/or double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, the nucleic acid molecule may comprise triple-stranded regions comprising RNA or DNA or both RNA and DNA. The nucleic acid molecule may also comprise one or more modified bases or DNA or RNA backbones modified for stability or for other reasons. A variety of modifications can be made to DNA and RNA; thus the term “nucleic acid molecule” embraces chemically, enzymatically, or metabolically modified forms.

In some embodiments of the invention, the nucleic acid molecule includes the nucleotide sequence set forth in SEQ ID NO: 52 or SEQ ID NO: 53.

It would be understood by a person skilled in the art that any nucleotide sequence which encodes a chimeric antigen receptor having the amino acid sequence set forth in SEQ ID NO: 52 or SEQ ID NO: 53, or a functional variant of SEQ ID NO: 52 or SEQ ID NO: 53, is contemplated by the present invention. For example, variants of SEQ ID NO: 52 or SEQ ID NO: 53 are contemplated which comprise one or more different nucleic acids to SEQ ID NO: 52 or SEQ ID NO: 53 but which still encode identical amino acid sequences. Because of the degeneracy of the genetic code, a large number of nucleic acids can encode any given protein. For instance, the codons GCA, GCC, GCG and GCU all encode the amino acid alanine. Therefore, at every position in SEQ ID NO: 52 or SEQ ID NO: 53 where an alanine is specified by a codon, the codon can be altered to any of the corresponding codons described without altering the encoded polypeptide. Accordingly, every nucleotide sequence herein which encodes a chimeric antigen receptor having the amino acid sequence set forth in SEQ ID NO: 52 or SEQ ID NO: 53, or a functional variant of SEQ ID NO: 52 or SEQ ID NO: 53 also describes every possible silent variation of the nucleotide sequence. One of skill will recognise that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleotide sequence that encodes a polypeptide is implicit in each described sequence.

Further, in at least some embodiments, the present invention provides a use of nucleic acid in the preparation of a vector for the transformation, transfection or transduction of a cell. Preferably, the cell is a T cell expressing one or more of CD3, CD4 or CD8. In some embodiments, the cell is used in the preparation of a medicament for the prevention or treatment of cancer. Consequently, is some embodiments, the present invention provides the use of a vector in the preparation of a medicament for the prevention or treatment of cancer.

It is to be understood that a nucleic acid construct, in accordance with the invention, may further comprise one or more of: an origin of replication for one or more hosts; a selectable marker gene which is active in one or more hosts; and/or one or more transcriptional control sequences.

As used herein, the term “selectable marker gene” includes any gene that confers a phenotype on a cell in which it is expressed, to facilitate the identification and/or selection of cells, which are transfected or transduced with the construct.

“Selectable marker genes” include any nucleotide sequences which, when expressed by a cell transduced with the construct, confer a phenotype on the cell that facilitates the identification and/or selection of these transduced cells. A range of nucleotide sequences encoding suitable selectable markers are known in the art (for example Mortesen, R M. and Kingston R E. Curr Protoc Mol Biol, 2009; Unit 9.5). Exemplary nucleotide sequences that encode selectable markers include: Adenosine deaminase (ADA) gene; Cytosine deaminase (CDA) gene; Dihydrofolate reductase (DHFR) gene; Histidinol dehydrogenase (hisD) gene; Puromycin-N-acetyl transferase (PAC) gene; Thymidine kinase (TK) gene; Xanthine-guanine phosphoribosyltransferase (XGPRT) gene or antibiotic resistance genes such as ampicillin-resistance genes, puromycin-resistance genes, Bleomycin-resistance genes, hygromycin-resistance genes, kanamycin-resistance genes and ampicillin-resistance gene; fluorescent reporter genes such as the green, red, yellow or blue fluorescent protein-encoding genes; and luminescence-based reporter genes such as the luciferase gene, amongst others which permit optical selection of cells using techniques such as Fluorescence-Activated Cell Sorting (FACS).

In some embodiments of the present invention, the selectable marker includes, or consists of, a modified surface expressed protein. In some embodiments, the surface expressed protein is the Epithelial Growth Factor Receptor (EGFR). In some embodiments, the Epithelial Growth Factor Receptor is truncated (EGFRt). In some embodiments, the selective marker is homologues to the sequence set forth in SEQ ID NO: 62, or a variant thereof having 50%, 60%, 70%, 80%, 90%, 95%, 98%, 99%, 99.5% or 99.7% sequence identity.

Furthermore, it should be noted that the selectable marker gene may be a distinct open reading frame in the construct or may be expressed as a fusion protein with another polypeptide (e.g. the CAR).

As set out above, the nucleic acid construct may also comprise one or more transcriptional control sequences. The term “transcriptional control sequence” should be understood to include any nucleic acid sequence which effects the transcription of an operably connected nucleic acid. A transcriptional control sequence may include, for example, a leader, polyadenylation sequence, promoter, enhancer or upstream activating sequence, and transcription terminator. Typically, a transcriptional control sequence at least includes a promoter. The term “promoter” as used herein, describes any nucleic acid which confers, activates or enhances expression of a nucleic acid in a cell.

In some embodiments, at least one transcriptional control sequence is operably connected to the nucleic acid molecule of the second aspect of the invention. For the purposes of the present specification, a transcriptional control sequence is regarded as “operably connected” to a given nucleic acid molecule when the transcriptional control sequence is able to promote, inhibit or otherwise modulate the transcription of the nucleic acid molecule. Therefore, in some embodiments, the nucleic acid molecule is under the control of a transcription control sequence, such as a constitutive promoter or an inducible promoter.

The “nucleic acid construct” may be in any suitable form, such as in the form of a plasmid, phage, transposon, cosmid, chromosome, vector, etc., which is capable of replication when associated with the proper control elements and which can transfer gene sequences, contained within the construct, between cells. Thus, the term includes cloning and expression vehicles, as well as viral vectors. In some embodiments, the nucleic acid construct is a vector. In some embodiments, the vector is a viral vector.

A promoter may regulate the expression of an operably connected nucleic acid molecule constitutively, or differentially, with respect to the cell, tissue, or organ at which expression occurs. As such, the promoter may include, for example, a constitutive promoter, or an inducible promoter. A “constitutive promoter” is a promoter that is active under most environmental and physiological conditions. An “inducible promoter” is a promoter that is active under specific environmental or physiological conditions. The present invention contemplates the use of any promoter which is active in a cell of interest. As such, a wide array of promoters would be readily ascertained by one of ordinary skill in the art.

Mammalian constitutive promoters may include, but are not limited to, Simian virus 40 (SV40), cytomegalovirus (CMV), P-actin, Ubiquitin C (UBC), elongation factor-1 alpha (EF1A), phosphoglycerate kinase (PGK) and CMV early enhancer/chicken β actin (CAGG).

Inducible promoters may include, but are not limited to, chemically inducible promoters and physically inducible promoters. Chemically inducible promoters include promoters which have activity that is regulated by chemical compounds such as alcohols, antibiotics, steroids, metal ions or other compounds. Examples of chemically inducible promoters include: tetracycline regulated promoters (e.g. see U.S. Pat. Nos. 5,851,796 and 5,464,758); steroid responsive promoters such as glucocorticoid receptor promoters (e.g. see U.S. Pat. No. 5,512,483), ecdysone receptor promoters (e.g. see U.S. Pat. No. 6,379,945) and the like; and metal-responsive promoters such as metallothionein promoters (e.g. see U.S. Pat. Nos. 4,940,661, 4,579,821 and 4,601,978) amongst others.

As mentioned above, the control sequences may also include a terminator. The term “terminator” refers to a DNA sequence at the end of a transcriptional unit which signals termination of transcription. Terminators are 3′-non-translated DNA sequences generally containing a polyadenylation signal, which facilitate the addition of polyadenylate sequences to the 3′-end of a primary transcript. As with promoter sequences, the terminator may be any terminator sequence which is operable in the cells, tissues or organs in which it is intended to be used. Suitable terminators would be known to a person skilled in the art.

As will be understood, the nucleic acid construct in accordance with the invention can further include additional sequences, for example sequences that permit enhanced expression, cytoplasmic or membrane transportation, and location signals. Specific non-limiting examples include an Internal Ribosome Entry Site (IRES).

The present invention extends to all genetic constructs essentially as described herein. These constructs may further include nucleotide sequences intended for the maintenance and/or replication of the genetic construct in eukaryotes and/or the integration of the genetic construct or a part thereof into the genome of a eukaryotic cell.

Methods are known in the art for the deliberate introduction (transfection/transduction) of exogenous genetic material, such as the nucleic acid construct of the third aspect of the present invention, into eukaryotic cells. As will be understood the method best suited for introducing the nucleic acid construct into the desired host cell is dependent on many factors, such as the size of the nucleic acid construct, the type of host cell the desired rate of efficiency of the transfection/transduction and the final desired, or required, viability of the transfected/transduced cells. Non-limiting examples of such methods include; chemical transfection with chemicals such as cationic polymers, calcium phosphate, or structures such as liposomes and dendrimers; non-chemical methods such as electroporation (see Potter and Heller. “Transfection by Electroporation.” Curr. Prot. Mol. Bio., ed. Frederick M. Ausubel et al. 2003: Unit-9.3), sonoporations (Wang, M et al. Sci Reps, 2018; 8: 3885), heat-shock or optical transfection; particle-based methods such as ‘gene gun’ delivery, magnetofection, or impalefection or viral transduction.

A variety of viral transduction techniques for mammalian cells are known in the art. Common viral vectors include lentivirus and retrovirus. An exemplary protocol is provided in Wang L et al., Proc. Natl. Acad. Sci, 2011; 108: E803-12. Alternative viral vectors include, HSV, Adenovirus and AAV (Howarth J et al. Cell. Bio. & Toxic., 2010, vol. 26, issue 1, pp 1-20).

In some embodiments, the present invention provide a lentivirus comprising a nucleic acid encoding a chimeric antigen receptor as described herein. Further, the present invention provides a use of the lentivirus in the preparation of a cell or a medicament for the prevention or treatment of cancer.

The nucleic acid construct will be selected depending on the desired method of transfection/transduction. In some embodiments, the nucleic acid construct is a viral vector, and the method for introducing the nucleic acid construct into a host cell is viral transduction. Methods are known in the art for utilising viral transduction to elicit expression of a CAR in a PBMC (Parker, L L. et al. Hum Gene Ther. 2000; 11: 2377-87) and more generally utilising retroviral systems for transduction of mammalian cells (Cepko, C. and Pear, W. Curr Protoc Mol Biol. 2001, unit 9.9). In some embodiments, the nucleic acid construct is a plasmid, a cosmid, an artificial chromosome or the like, and can be transfected into the cell by any suitable method known in the art.

Nucleic acid constructs in accordance with the invention can be used to generate genetically modified cells which can be used for the killing of target cells expressing a dysfunctional P2X7 receptor. Cells suitable for genetic modification can be heterologous or autologous.

Techniques are known in the art for selection/isolation of cell subsets. These include Fluorescent Activated Cell Sorting (Basu S. et al. J. Vis. Exp. 2010; 41: 1546), techniques utilising antibodies immobilised on a substrate, such as magnetic cell isolation (MACS®) device to immunomagnetically select cells expressing the desired markers (Zola H. et al. Blood, 2005; 106(9): 3123-6), or use of microfluidic chips. A series of cell markers can be used to isolate cells of the immune system including (but not limited to), BCR, CCR10, CD1a, CD1b, CD1c, CD1d, CD3, CD4, CD5, CD7, CD8, CD10, CD11b, CD11c, CD13, CD16, CD19, CD21, CD23, CD25, CD27, CD31, CD32, CD33, CD34, CD38, CD39, CD40, CD43, CD45, CD45RA, CD45RO, CD48, CD49d, CD49f, CD51, CD56, CD57, CD62, CD62L, CD68, CD69, CD62, CD62L, CD66b, CD68, CD69, CD73, CD78, CD79a, CD79b, CD80, CD81, CD83, CD84, CD85g, CD86, CD94, CD103 CD106, CD115, CD117, CD122, CD123, CD126, CD127, CD130, CD138, CD140a, CD140b, CD141, CD152, CD159a, CD160, CD161, CD163, CD165, CD169, CD177, CD178, CD183, CD185, CD192, CD193, CD194, CD195, CD196, CD198, CD200, CD200R, CD203c, CD205, CD206, CD207, CD209, CD212, CD217, CD218 alpha, CD229, CD244, CD268, CD278, CD279, CD282, CD284, CD289, CD294, CD303, CD304, CD314, CD319, CD324, CD335, CD336, CXCR3, Dectin-1, Tc epsilor R1 alpha, Flt3, Granzyme A, Granzyme B, IL-9, IL-13apha1, IL-21R, iNOS, KLRG1, MARCO, MHC class II, RAG, ROR Gamma T, Singlec-8, ST2, TCR alpha/beta, TCR gamma/delta, TLR4, TLR7, VEGF, ZAP70

Of particular note are the T cell markers CCR10, CD1a, CD1c, CD1d, CD2, CD3, CD4, CD5, CD7, CD8, CD9, CD10, CD11 b, CD11c, CD13, CD16, CD23, CD25, CD27, CD31, CD34, CD38, CD39, CD43, CD45, CD45RA, CD45RO, CD48, CD49d, CD56, CD62, CD62L, CD68, CD69, CD73, CD79a, CD80, CD81, CD83, CD84, CD86, CD94, CD103, CD122, CD126, CD127, CD130, CD140a, CD140b, CD152, CD159a, CD160, CD161, CD165, CD178, CD183, CD185, CD192, CD193, CD194, CD195, CD196, CD198, CD200, CD200R, CD212, CD217, CD218 alpha, CD229, CD244, CD278, CD279, CD294, CD304, CD314, CXCR3, Flt3, Granzyme A, Granzyme B, IL-9, IL-13alpha1, IL-21R, KLRG1, MHC class II, RAG, ROR gamma T, ST2, TCR alpha/beta, TCR gamma/delta, ZAP70. Particularly preferred cell markers for T cell selection include TCRgamma, TCR delta, CD3, CD4 and CD8.

Isolated cells can then be cultured to modify cell activity, expanded or activated. Techniques are known in the art for expanding and activating cells (Wang X. and Rivière I. Mol. Thera. Oncolytics. 2016; 3: 16015). These include; using anti-CD3/CD28 microbeads, or other forms of immobilised CD3/CD28 activating antibodies. Activated/genetically modified cells can then be expanded in vitro in the presence of cytokines (such as with IL-2, IL-12, IL-15 or IL-17) and then cryopreserved. An overview of methods for expanding CAR T cells is provided in Wang and Riviera ibid).

The present invention further provides a genetically modified cell including the chimeric antigen receptor, nucleic acid molecule, or nucleic acid construct as described above. In some embodiments, the genetically modified cell is a leukocyte. In some embodiments, the genetically modified cell is a Peripheral Blood Mononuclear Cell (PBMC). In some embodiments, the genetically modified cell is a myeloid cell. In some embodiments, the genetically modified cell is a monocyte. In some embodiments, the genetically modified cell is a macrophage. In some embodiments, the genetically modified cell is a lymphocyte. In some embodiments, the genetically modified cell is a T cell. In some embodiments, the genetically modified cell is an alpha beta (αβ) T cell. In some embodiments, the genetically modified cell is a gamma delta (γδ) T cell. In some embodiments, the genetically modified cell is a virus-specific T cell. In some embodiments, the genetically modified cell is a CD3+ T cell (such as a naive CD3+ T cells or a memory CD3+ T cell subsets). In some embodiments, the T cell is a CD4+ T cell (such as a naive CD4+ T cells or a memory CD4+ T cell subsets). In some embodiments, the T cell is a CD8+ T cell (such as a naive CD8+ T cells or a memory CD8+ T cell subsets). In some embodiments, the genetically modified cell is a natural killer cell. In some embodiments, the genetically modified cell is a natural killer T cell.

### Use of Chimeric Antigen Receptor Expressing Cells

Genetic modified cell can be used to target cells expressing dysfunctional P2X7 receptors, and (depending on the cell type) may assist in, or lead to, killing of the cell expressing the dysfunctional receptor. In some embodiments, the present invention provides a method of killing a cell expressing a dysfunctional P2X7 receptor, the method including contacting the cell expressing the dysfunctional P2X7 receptor, with a genetically modified cell expressing a chimeric antigen receptor as described above.

The cell expressing the dysfunctional P2X7 receptor may be a cancer cell. Therefore, in some embodiments, the present invention provides a use of a genetically modified cell as described above for treating cancer. Furthermore, the invention provides a method of killing a cell expressing a dysfunctional P2X7 receptor, the method comprising contacting the cell expressing a dysfunctional P2X7 receptor with a cell including a nucleic acid molecule or nucleic acid construct, as described above. In some embodiments, the cells expressing a dysfunctional P2X7 receptor is a cancer cell.

In some embodiments, the present invention provides a method of killing a cell expressing a dysfunctional P2X7 receptor, the method including contacting the cell expressing the dysfunctional P2X7 receptor with a genetically modified cell expressing a chimeric antigen receptor as described above.

In some embodiments, the cancer cell is a solid cancer cell. In some embodiments, the cancer cell is selected from the group consisting of: brain cancer cell, oesophageal cancer cell, mouth cancer cell, tongue cancer cell, thyroid cancer cell, lung cancer cell, stomach cancer cell, pancreatic cancer cell, kidney cancer cell, colon cancer cell, rectal cancer cell, prostate cancer cell, bladder cancer cell cervical cancer cell, epithelial cell cancers, skin cancer cell, leukaemia cell, lymphoma cell, myeloma cell, breast cancer cell, ovarian cancer cell, endometrial cancer cell and testicular cancer cell. In some embodiments, the cancer cell is selected from the group consisting of: a breast cancer cell, a glioblastoma cancer cell, an ovarian cancer cell, or a melanoma cancer cell. In some embodiments, the cancer cell is from a metastatic cancer. In some embodiments, the cancer is stage III cancer or is stage IV cancer

In some embodiments, the genetically modified cell is autologous to the cell expressing a dysfunctional P2X7 receptor. In some embodiments, the cell expressing a dysfunctional P2X7 receptor is within the body of a subject.

In some embodiments the chimeric antigen receptor according to the present invention, when expressed in a CD8+ cytotoxic T lymphocyte (CTL), has cytotoxicity in vitro against Target cells expressing a dysfunctional P2X7 receptor of at least 20%, at least 30%, at least 40% or at least 50% at a ratio of CAR Transduced CTL: target cells of 30:1 or greater, 10:1 or greater, 3:1 or greater or 1:1 or greater.

In some embodiments, the chimeric antigen receptor of the invention, when expressed in a CD3+ T cell, demonstrates activity against as least 2 different cancer types, at least 3 different cancer types, at least 4 different cancer types, at least 5 different cancer types, at least 6 different cancer types, at least 7 different cancer types, at least 8 different cancer types, at least 9 different cancer types, at least 10 different cancer types.

In some embodiments, the chimeric antigen receptor according to the present invention, when expressed in a CD4+ T-helper cell, increase IL-2, TNF alpha and/or IFN gamma production when co-cultured with a target cell expressing a dysfunctional P2X7 receptor. In some embodiments, the increase is a statistically significant increase. In some embodiments the statistically significant increase is to a P-value of 0.05, 0.01 or 0.001.

In some embodiments, the cells expressing a dysfunctional P2X7 receptor are cancer cells.

The present invention further provides the use of a chimeric antigen receptor as described herein, when expressed in an immune cell, for treating a cancer. In some embodiments, the immune cell a Peripheral Blood Mononuclear Cell (PBMC). In some embodiments, the immune cell is a myeloid cell. In some embodiments, the immune cell is a monocyte. In some embodiments, the immune cell is a macrophage. In some embodiments, the immune cell is a lymphocyte. In some embodiments, the immune cell is a natural killer cell. In some embodiments, the immune cell is a natural killer T cell. In some embodiments, the immune cell is a T cell. In some embodiments, the genetically modified cell is a gamma delta (γδ) T cell. In some embodiments, the genetically modified cell is a virus-specific T cell. In some embodiments, the immune cell is a CD3+ T cell (such as a naive CD3+ T cells or a memory CD3+ T cell subsets). In some embodiments, the T cell is a CD4+ T cell (such as a naive CD4+ T cells or a memory CD4+ T cell subsets). In some embodiments, the T cell is a CD8+ T cell (such as a naive CD8+ T cells or a memory CD8+ T cell subsets).

The present invention also provides a pharmaceutical composition including a genetically modified cell including a chimeric antigen receptor, a nucleic acid molecule or a nucleic acid construct as described above.

T cells or other immune cells modified to express a chimeric antigen receptor described herein may be formulated into a pharmaceutical composition along with a “carrier” or “excipients” for delivery to a subject. As used herein, “carrier” or “excipient” includes any solvent, dispersion medium, vehicle, coating, diluent, antibacterial, and/or antifungal agent, isotonic agent, absorption delaying agent, buffer, suspension, colloid, or the like. The use of such media and/or agents for pharmaceutical active substances is well known in the art. Except insofar as any conventional media or agent is incompatible with genetically modified cells, its use in the therapeutic compositions is contemplated. Supplementary active ingredients also can be incorporated into the compositions. By “pharmaceutically acceptable” is meant a material that is not biologically undesirable, or undesirably reactive or toxic, and the material may be administered to an individual along with genetically modified cells expressing a chimeric antigen receptor without causing any undesirable biological effects or interacting in a deleterious manner with any of the other components of the pharmaceutical composition (particularly the genetically modified cells) in which it is contained.

The pharmaceutical composition may be formulated in a variety of forms adapted to a preferred route of administration. Thus, a composition can be administered via known routes including, for example, parenteral (e.g., intradermal, transcutaneous, subcutaneous, intramuscular, intravenous, intraperitoneal, etc.). A composition also can be administered via a sustained or delayed release.

A formulation may be conveniently presented in unit dosage form and may be prepared by methods well known in the art of pharmacy. Methods of preparing a composition with a pharmaceutically acceptable carrier include the step of bringing genetically modified cells expressing a chimeric antigen receptor into association with a carrier that constitutes one or more accessory ingredients. A pharmaceutical composition that includes genetically modified cells expressing a chimeric antigen receptor may be provided in any suitable form, including, but not limited to, a solution, a suspension, an emulsion, a spray, an aerosol, or any form of mixture.

The composition may be delivered in formulation with any pharmaceutically acceptable excipient, carrier, adjunct or vehicle. In some embodiments, the pharmaceutical composition that genetically modified cells expressing a chimeric antigen receptor may be administered, for example, from a single dose to multiple doses per week. In some embodiments, the method can be performed by administering the pharmaceutical composition at a frequency outside this range.

In some embodiments, the pharmaceutical composition may be administered from about once to about five times per week. In some embodiments the pharmaceutical composition is administered once. In some embodiments, the pharmaceutical composition is administered twice. In some embodiments, the pharmaceutical composition is administered three times. In some embodiments, the pharmaceutical composition is administered four times.

In some embodiments, the pharmaceutical composition includes at least 5×10⁸ cells. In some embodiments, the pharmaceutical composition includes at least 3×10⁸ cells. In some embodiments, the pharmaceutical composition includes at least 2.5×10⁸ cells. In some embodiments, the pharmaceutical composition includes at least 1×10⁸ cells. In some embodiments, the pharmaceutical composition includes at least 5×10⁷ cells. In some embodiments, the pharmaceutical composition includes at least 2.5×10⁷ cells. In some embodiments, the pharmaceutical composition includes at least 1×10⁷ cells. In some embodiments, the pharmaceutical composition includes at least 5×10⁶ cells. In some embodiments, the pharmaceutical composition includes at least 2.5×10⁶ cells. In some embodiments, the pharmaceutical composition includes at least 1×10⁶ cells.

In some embodiments, the pharmaceutical composition is administered to provide at least 5×10⁸ cells. In some embodiments, the pharmaceutical composition is administered to provide at least 3×10⁸ cells. In some embodiments, the pharmaceutical composition is administered to provide at least 2.5×10⁸ cells. In some embodiments, the pharmaceutical composition is administered to provide at least 1×10⁸ cells. In some embodiments, the pharmaceutical composition is administered to provide at least 5×10⁷ cells. In some embodiments, the pharmaceutical composition is administered to provide at least 2.5×10⁷ cells. In some embodiments, the pharmaceutical composition is administered to provide at least 1×10⁷ cells. In some embodiments, the pharmaceutical composition is administered to provide at least 5×10⁶ cells. In some embodiments, the pharmaceutical composition is administered to provide at least 2.5×10⁶ cells. In some embodiments, the pharmaceutical composition is administered to provide at least 1×10⁶ cells.

Generally, the pharmaceutical composition is administered to a subject in an amount, and in a dosing regimen effective to reduce, limit the progression of, ameliorate, or resolve, to any extent, the symptoms or clinical signs of a condition such as cancer. As used herein, “ameliorate” refers to any reduction in the extent, severity, frequency, and/or likelihood of a symptom or clinical sign characteristic of cancer. “Symptom” refers to any subjective evidence of disease or of a patient's condition. “Sign” or “clinical sign” refers to an objective physical finding relating to a particular condition capable of being found by one other than the patient. In the context of cancer, the composition is administered to a subject in an amount, and in a dosing regimen effective to limit the growth of one or more tumours, reduce the size, volume or weight of one or more tumours, reduce the rate metastasis of the cancer or number of metastases, reduce the proliferation of cancer cells, or extend the life expectancy of a subject.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

Finally, reference is made to standard textbooks of molecular biology that contain methods for carrying out basic techniques encompassed by the present invention. See, for example, Green MR and Sambrook J, Molecular Cloning: A Laboratory Manual (4th edition), Cold Spring Harbor Laboratory Press, 2012.

It will be apparent to the person skilled in the art that while the invention has been described in some detail for the purposes of clarity and understanding, various modifications and alterations to the embodiments and methods described herein may be made without departing from the scope of the inventive concept disclosed in this specification.

The invention is further illustrated in the following examples. The examples are for the purpose of describing particular embodiments only and are not intended to be limiting with respect to the above description

### EXAMPLE 1

### Preparation and Expression of anti-Dysfunctional (non-functional) P2X7 Chimeric Antigen Receptor.

### Anti-Dysfunctional P2X7 CAR Constructs

An exemplified protocol detailing the process of designing and expressing an anti-dysfunctional P2X7 receptor chimeric antigen receptor according to an embodiment of the present invention is detailed as follows.

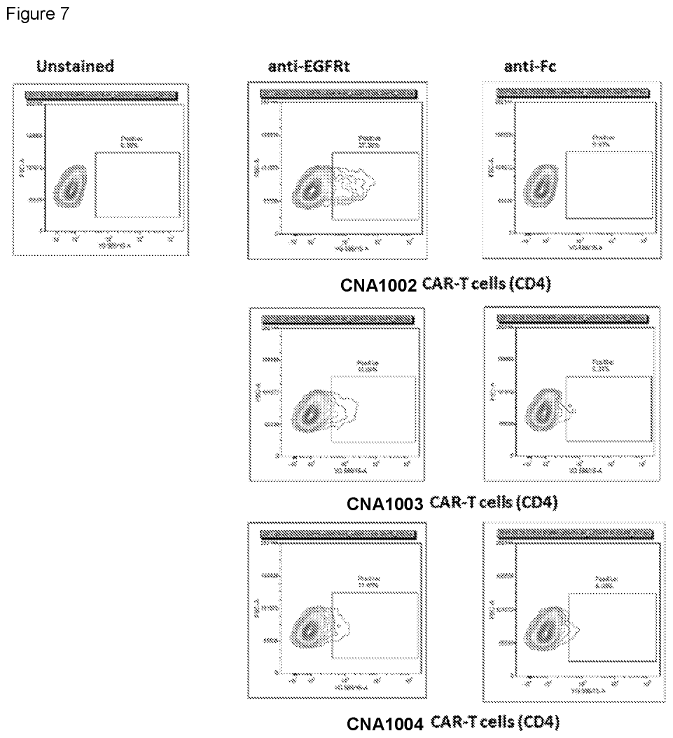

CAR constructs (collectively referred to as CNA CAR family constructs) were prepared as illustrated in FIG. 7 comprising: (i) an ectodomain 1 comprising a CSF2RA (human colony stimulating factor 2 receptor alpha) leader sequence 2, antigen binding domain 3 directed against dysfunctional P2X7 receptors having a trans-conformation to cis-conformational change to the proline at amino acid position 210 (having sequence homology to V_{H} portion of an antibody), (ii) a CD28 transmembrane domain 4, and (iii) an endodomain 5 comprising the intracellular portion of 41BB 6, the intracellular portion of the CD3-zeta chain 7, a T2A self-cleavage site 8, and a truncated form of the EGFR receptor (EGFRt) 9 that lacks an intracellular signalling domain (the surface expression of EGFRt can be used as a proxy to measure transduction efficiency).

The ectodomain 1 and the transmembrane domain 4 were linked by one of three linked domains being:
a. A linker region of 12 amino acids 10, comprising a mutated version of the IgG4 hinge region (see FIG. 2 and SEQ ID NO: 13 and 38)—generating a CAR referred to as CNA1002. The amino acid sequence and nucleic acid sequence for this chimeric antigen receptor is provided by SEQ ID NO: 54 and 55, respectively;
b. A linker region of 119 amino acids 11, comprising a mutated version of the IgG4 hinge region and the IgG4 CH2 region (see SEQ ID NO: 39)—generating a CAR referred to as CNA1003. The amino acid sequence and nucleic acid sequence for this chimeric antigen receptor is provided by SEQ ID NO: 51 and 53, respectively; and
c. A linker region of 228 amino acids 12, comprising a mutated version of the IgG4 hinge region, the IgG4 CH2 region and the IgG4 CH3 region (see SEQ ID NO: 40)—generating a CAR referred to as CNA1004. The amino acid sequence and nucleic acid sequence for this chimeric antigen receptor is provided by SEQ ID NO: 56 and 57, respectively.

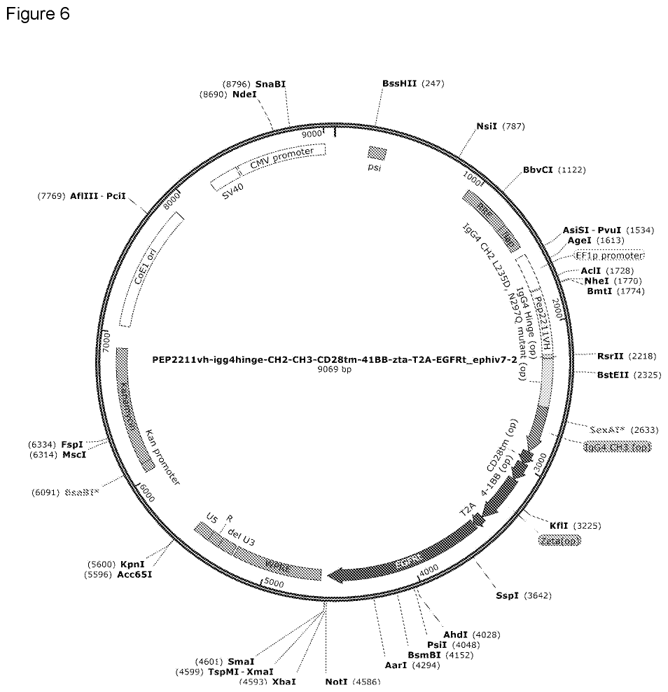

These nucleic acid molecules were then cloned into a lentiviral backbone (epHIV −7.2—FIG. 6) to form a nucleic acid construct.

A further family of CARs were constructed (SEQ ID NO: 60 and SEQ ID NO: 61) were prepared comprising a CD8a signalling peptide 13, an anti-dysfunctional P2X7 binding peptide 14 (referred to as PEP2-2-3) distinct from binding peptide used in the CNA family described above, a transmembrane region comprising a portion of CD28 15, which also provided a portion of the endodomain 5, and an intracellular portion comprising an intracellular portion of OX40 16 and an intracellular portion of the CD3 zeta chain 17, and a T2A self-cleavage site 9. The binding peptide and the transmembrane region were linked by a linking domain of 30 amino acids (SEQ ID NO: 41) 18 or 228 amino acids 19 (SEQ ID NO 63). The 30 amino acid linker domain, comprised a mutated version of the IgG4 hinge region (12 a.a.) proceeded by the linker (G₄5)₃ (15 a.a.) and followed by the amino acid sequence “DPK” (Referred to as BLIV CAR short hinge linker—see SEQ ID NO: 41

### CNA Family Viral Transfection and Lentiviral Production

239 T cells were transiently transfected with vectors containing the CNA family of CARs to produce lentivirus according to the following protocol:

Day 1—293T cells were seeded in 10 ml of DMEM media supplemented with 10% serum in a 10 cm cell culture plate. Substantial confluence was achieved by 24 hours. Four plates were prepared for packaging each virus. The plates were incubated overnight at 37° C. with 5% CO₂ to allow the cells to adhere to the plates.

Day 2—The reagent and plasmid DNA amounts used for transient transfection of four plates are listed below (Table 6) using Lipofectamine 2000 (Invitrogen). Virus was made for the three different CAN CAR constructs: CNA1002, CNA1003 and CNA1004.

Two tubes were prepared for each virus. In tube one: the LV-CAR encoding plasmid was combined with three viral packaging plasmids: (pCMV-Rev2, pCHGP-2, and pCMV-G) was diluted in OptiMEM media. In tube two: appropriate volume of Lipofectamine 2000 reagent (Invitrogen) was diluted in OptiMEM.

The contents of the two tubes were combined, gently agitated, prior to incubation for 20 minutes at room temperature. The mixture (1 ml per plate) was then added to the 293T cells prepared on day one and incubated at 37° C. with 5% CO₂ overnight.

TABLE 6
Viral packaging transfection

| | | Amount | | | | | | |
|---|---|---|---|---|---|---|---|---|
| | | of DNA | | Total | DNA dilution | | Liposome dilution | |
| | DNA | per | | amount | Vol of | Vol of | Vol of | Vol of |
| | conc | plate | No of | of DNA | DNA | OptiMEM | LP2000 | OptiMEM |
| Vector | (ug/ul) | (ug) | plates | (ug) | (ul) | (ul) | (ul) | (ul) |
| Plasmid | 1 | 15 | 4 | 60 | 60 | 2000 | 160 | 2000 |
| of interest | | | | | | | | |
| (LV-CAR | | | | | | | | |
| construct) | | | | | | | | |
| pCMV- | 1 | 1 | 4 | 4 | 4 | | | |
| Rev2 | | | | | | | | |
| (pj1505) | | | | | | | | |
| pCHGP-2 | 1 | 10 | 4 | 40 | 40 | | | |
| (pj1506) | | | | | | | | |
| pCMV-G | 1 | 2 | 4 | 8 | 8 | | | |
| (pj1507) | | | | | | | | |

Day 3—The media was removed, and replaced with fresh DMEM media supplemented with 10% serum and sodium butyrate (at a final concentration of 6 mM), followed by incubation for a further 48 hours at 37° C. with 5% CO₂.

Day 5—Supernatant from the four plates was collected and transferred to conical tubes before being spun at 914 RCF for 10 minutes, to remove cellular debris. The supernatant was then filtered (0.45 μM) and virus was concentrated from the filtered supernatant by centrifugation.

After the centrifugation, the supernatant was discarded and any remaining media was allowed to drain out by inverting the tubes. The remnant virus pellet was resuspended in 200 μl of serum free DMEM and the concentrated virus suspension was then transferred to a new tube. The virus was further concentrated by high speed centrifugation, prior to storage at −80° C.

### Viral Titre Assay

To determine the transduction capacity of the prepared viruses, known volumes of the concentrated viruses (1:10000, 1:5000, 1:2000, 1:1000, 1:500. 1:166 and 1:100) was incubated with H9 cells (1×10⁵ in 500 μl of media) in the presence of protamine sulphate (0.1 mg/ml) for 48 h at 37° C. with 5% CO₂. The titre for each virus was determined by staining the transduced H9 cells with anti-EGFR (Erbitux-Biotin) and calculating the surface expression by flow cytometry.

### Preparation of Genetically Modified CAR Expressing Cells.

Transduced CD3+, CD4+ and CD8+ cells were prepared by one of the following protocols:

### Protocol 1

CD4+ and CD8+ T cells were isolated from discarded leukoreduction chambers (LRS chamber) from a platelet apheresis kit (Bloodworks NW). The T cells were either isolated using AutoMACs Pro® separator or LS columns.

CD4+ and CD8+ T cells were separately transduced with each of the three CNA CAR-viruses (CNA1002, CNA1003 and CNA1004). An un-transduced mock well was included for each cell type as a control.

Cells were stimulated with CD3/CD28 Dynabeads® (1:1) for 1-3 days after isolation. The stimulated cells were counted and plated in a 24-well plate (2×10⁶ cells per well) in 500 ul of media. Protamine sulfate was added to each well at a final concentration of 40 ug/ml.

The amount of virus needed for transduction was calculated based on the viral titer results obtained from the method described above. A multiplicity of infection (MOI) of 3 was used to transduce the CD4+ and CD8+ cells. After addition of the thawed virus suspension, the plate was swirled to mix and spinoculated (800 RCF) for 30 minutes at 32° C. The plates were then incubated for 4 h at 37° C. with 5% CO₂. After the incubation warm complete medium (1.5 ml per well) supplemented with 5 ng/ml rhIL-7 and 0.5 ng/ml rhIL15 for CD4+ cells, and 50 u/ml rhII-2 and 0.5 ng/ml rhIL-15 for CD8, was added to each well.

Transduced cells were maintained by replenishing half of the cytokine containing media every 2-3 days. Culture volumes were expanded when cells became visually crowded. Dynabeads were removed after day 9 of stimulation.

### Protocol 2

CD3+, CD4+ and CD8+ T cells were isolated from whole blood using Rosettesep Human CD4, CD8 or CD3 T cell enrichment cocktail (StemCell), following the manufacture's protocol.

CD3+, CD4+ and CD8+ T cells were separately transduced with each of the three CNA CAR-viruses (CNA1002, CNA1003 and CNA1004). Additionally, an un-transduced mock well was included for each cell type as a control.

CD4, CD8 and CD3 cells were cultured in complete ex vivo media supplemented with the following cytokines (Table 7)

TABLE 7
Cytokine supplements

| Cell Type | IL-2 | IL-7 | IL-15 |
|---|---|---|---|
| CD4 | 50 U/ml | 5 ng/ml | 0.5 ng/ml |
| CD8 | 50 U/ml | 5 ng/ml | 5 ng/ml |
| CD3 | 50 U/ml | 5 ng/ml | 0.5 ng/ml |

Cells were stimulated with CD3/CD28 Dynabeads for 1 h after isolation (3:1 cell to bead ratio). The stimulated cells were transduced at a multiplicity of infection (MOI) of 20 with polybrene at a final concentration of 8 ug/ml. The plates were then incubated overnight at 37° C. with 5% CO2.

On Day 2, half the media was removed and replaced to dilute the concentration of polybrene. Dynabeads® were removed from CD8 cells after a 24 h stimulation. In CD3+ and CD4+ T cells Dynabeads® were left on for 10 days. Transduced cells were fed every 2-3 days by removing half media and adding fresh media supplemented with appropriate cytokines. Culture volumes were expanded when cells became visually crowded.

### Determining Transduction Efficiency

After 10 days of stimulation transduction efficiency was determined by staining the transduced CD3+, CD4+ and CD8+ cells for EGFR and/or Fc expression in accordance with the following protocol:
A sample of the transduced cells were transferred to 5 ml polystyrene tubes and spun at 1200 rpm for 3 minutes at room temperature to form a pellet. The supernatant was removed and the cell pellet was washed with 2 ml of FACS staining solution;
Three samples were prepared for each transduced cell line (i) unstained control tube, (ii) anti-EGFR stained cells, and (iii) anti-Fc stained cells;
Cells (excluding the unstained control cells) were incubated with a 1:100 dilution of biotinylated primary antibodies (anti-EGFR or anti-Fc) for 20 minutes at room temperature in the dark followed by washing the cells by centrifuging as above, and washing the obtained pellet with 2 ml of FACS staining solution (x2);
Washed cells were incubated with a PE-conjugated streptavidin secondary antibody for 20 minutes at room temperature in the dark, followed by washing as outlined above;
The washed cells were then centrifuged and resuspended in 200 ul of FACS fixative and stored at 4° C. until analysis by flow cytometry.

The results for CD4+ cells are provided in FIG. 7. 27.5% of CD4+ cells transduced with CNA1002 CAR were positive for truncated EGFR (EGFRt) expression indicating transduction with the CNA1002 CAR. 16.09% of CD4+ cells transduced with CNA1003 were positive for EGFRt expression, and 11.65% of CD4+ cell transduced with CNA1004 were positive for EGFRt.

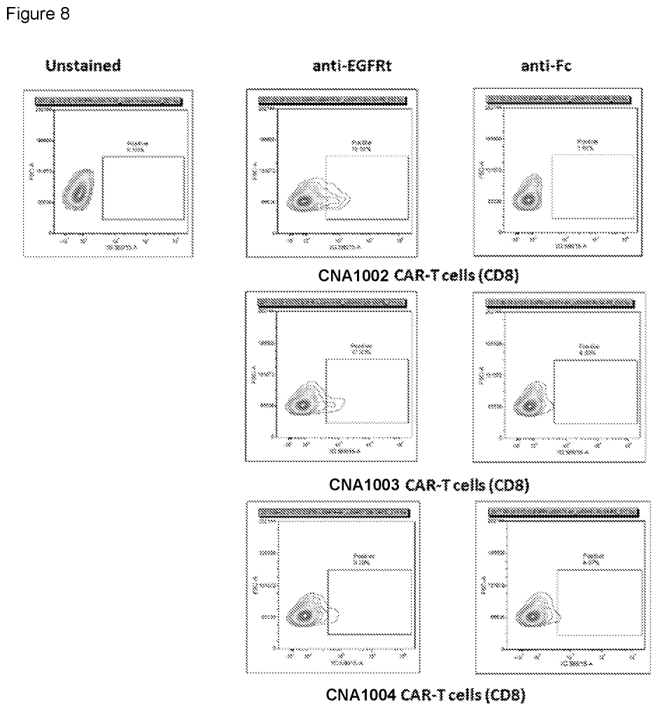

The results for CD8+ cells are provided in FIG. 8. 9.84% of CD8+ cells transduced with CNA1002 CAR were positive for truncated EGFR (EGFRt) expression indicating transduction with the CNA1002 CAR. 12.30% of CD8+ cells transduced with CNA1003 were positive for EGFRt expression, and 9.20% of CD8+ cells transduced with CNA1004 were positive for EGFRt.

### Cell Sorting and Expansion

To increase purity of transduced cells, EGFRt expressing cells were isolated using positive selection and magnetic beads. Transduced CD4+ and CD8+ cells were stained with biotinylated anti-EGFR antibody (1:100) for 20 minutes at 4° C., then washed as described above, and incubated with anti-Biotin Microbeads (Miltenyi®) for 15 minutes at 4° C. Cells were sorted using a MidiMACS magnet and LS columns in accordance with the manufacturer's protocol.

Alternatively, transduced cells were purified by Fluorescent activated cell sorting (FACS) following staining with a labelled anti-EGFR antibody using protocols known in the art.

Purified transduced cells were subsequently expanded for 12 days by stimulating the cells using irradiated feeder cells (PBMC and transduced B cells) and soluble OKT3 antibody. In a T25 flask CD3+, CD4+ and CD8+ CAR-T cell were incubated with frozen PBMC at a ratio of 1:50 or 1:25 (T cell:PBMC). Transduced B cell line (1×10⁶) and/or soluble OKT3 (anti-CD3_antibody (30 ng/ml) was also added in 25 ml complete RPMI. Additionally, rhIL-7 and rhIL-15 were added to CD4+ cells and rhIL-2 and rhII-15 were added to CD8+ Transduced cells were maintained by replenishing half of the media every 2 to 3 days. Culture volumes were expanded when cells became visually crowded.

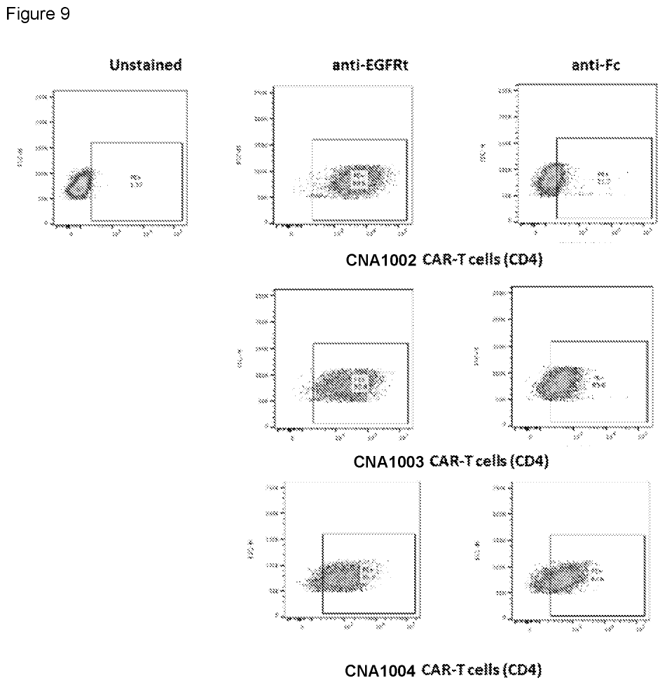

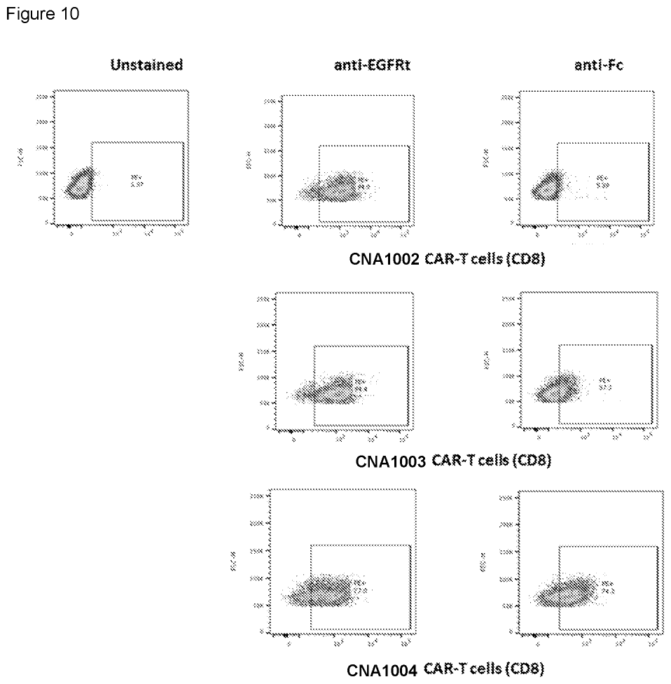

After the 12 days of expansion, the transduced CD4+ and CD8+ cells were analysed for EGFRt and CART surface expression by flow cytometry using anti-EGFR and anti-human Fc antibodies as described above (FIGS. 9 and 10).

The results for CD4+ cells are provided in FIG. 9. 99.5% of CD4+ cells transduced with CNA1002 CAR were positive for truncated EGFR (EGFRt) expression indicating transduction with the CNA1002 CAR. 99% of CD4+ cells transduced with CNA1003 were positive for EGFRt expression, and 82.9% of CD4+ cell transduced with CNA1004 were positive for EGFRt. 21.2% of CAN1002 transduced CD4+ cells were positive for Fc, 83.6% of CNA1003 transduced CD4+ cells were positive for Fc and 82.6% of CNA1004 transduced CD4+ cells were positive for Fc.

The results for CD8+ cells are provided in FIG. 10. 94% of CD8+ cells transduced with CNA1002 CAR were positive for truncated EGFR (EGFRt) expression indicating transduction with the CNA1002 CAR. 94.4% of CD8+ cells transduced with CNA1003 were positive for EGFRt expression, and 77% of CD8+ cells transduced with CNA1004 were positive for EGFRt. 5.39% of CNA1002 transduced CD8+ cells were positive for Fc, 37.3% of CNA1003 transduced CD8+ cells were positive for Fc and 74.2% of CNA1004 transduced CD8+ cells were positive for Fc.

### EXAMPLE 2

### Anti Dysfunctional-P2X7 Chimeric Antigen Receptor Effector Function

To assess the functionality of CNA1002, CNA1003 and CNA1004 transduced T cells, in vitro killing assays (CD8+ cells) and cytokine release assays (CD4+ cells) were performed as described below

CD8+ CART effector function

CD8+ transduced T cells expressing the three CNA family CARs (CNA1002, CNA1003, CNA1004) were assessed for cytotoxic activity using a chromium release assays.

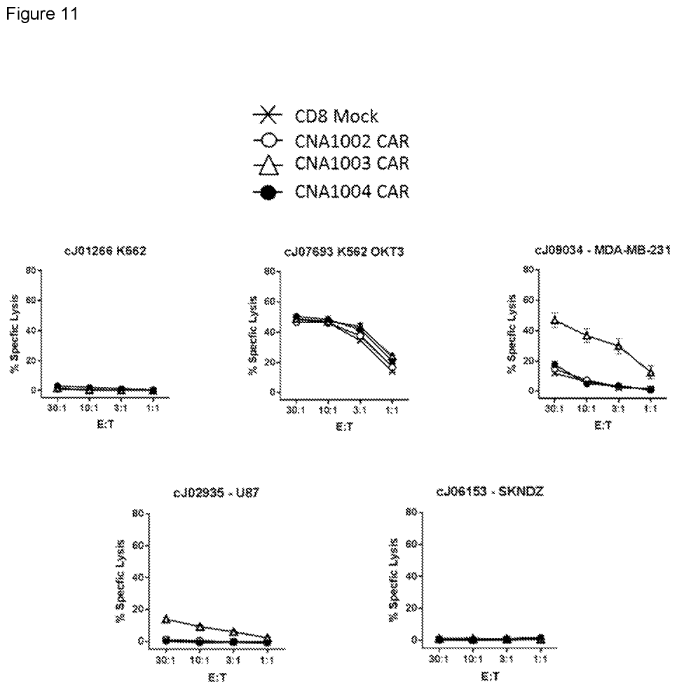

A first functional assay (FIG. 11) was performed against a series of target cells (both adherent cells and non-adherent cells) and a positive control cell line (K562 cells expressing OKT3) and a non-cancerous control cell line (K562 cell line). Three cancer cell lines: MDA-MB-231 (breast cancer), U87 (glioma) and SKNDZ (neuroblastoma) cell lines were used as target cells.

Step 1—Day 1—Adherent MDA-MB-231 (breast cancer cell line) target cells (×10⁶) were seeded into a T75 flask in 7 ml of complete media (DMEM with 10% FBS) in the morning and incubated at 37° C. with 5% CO₂ for 6 hrs to allow adherence to the flask. Following adherence, ⁵¹Cr (75 ul of 5 mCi/ml) was added to each flask, mixed, and incubated at 37° C. (5% CO₂) overnight.

Non-adherent target cell lines (K562, K562 expressing OKT3, 293T, U87 and SKNDZ), were seeded (5×10⁶ per well) into 12-well plates in 4 ml of complete media. 75 ul of 5 mCi/ml ⁵¹Cr was added to each well, mixed, and incubated at 37° C. (5% CO₂) overnight.

Step 2—Day 2—CD8+ transduced cells expressing the CNA family CARs were counted and cell volumes needed for the 4 different dilutions (30:1, 10:1, 3.3:1, 1.1:1) were calculated.

Step 3—The ⁵¹Cr labelled target cells (adherent cells were collected by trypsinisation) were washed twice with PBS (10 ml) solution before counting. Cell concentrations were adjusted to 5×10⁴/ml and 5000 target cells in complete RPMI (with 10% FBS) were added to each well containing effector cells to provide the desired effector to target (E:T) ratio.

Additional control wells were plated for each target cell line. For the maximum cytolysis wells, 100 ul of 2% SDS solution was added to target cells alone, to cause complete cell lysis. For minimum cytolysis, or background radiation, complete media was added. Once effectors and targets were combined the plates were incubated at 37° C. with 5% CO₂ for 4 hours.

At the end of the 4 hours incubation the assay plates were spun at 10⁴ RCF at room temperature with brakes. Then 50 ul of the supernatant was harvested and transferred to white LUMA plates. The LUMA plates were allowed dry overnight on the bench.

Step 4—Day 3—Each LUMA plate was assessed for ⁵¹Cr (counts per minute—CPM), indicative of cell killing, with a TopCount scintillation counter (Perkin Elmer), and the percentage of cytolysis was calculated as set out below.

Percentage cytolysis=(CPMₛₐₘₚₗₑ−CPM_{Min})/(CPM_{Max}−CPM_{Min})×100

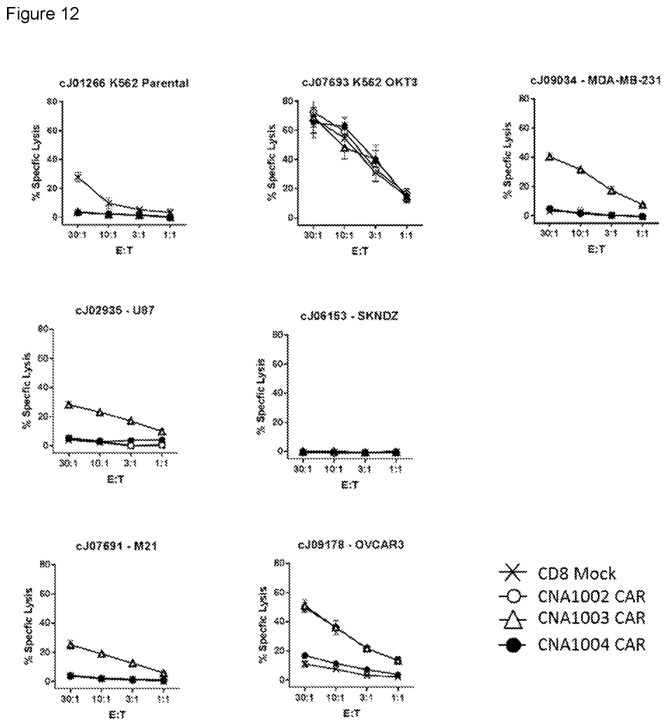

A second functional assay (FIG. 12) was performed as described above, which included the above target cells and M21-melanoma cells and OVCAR3-ovarian cancer cells.

As can be seen in FIG. 11, the first functional assay demonstrated that CNA1003 expressing CD8+ CAR-T cells showed specific cytolysis against two cancer cell lines, MDA-MB-231 (46% E:T 30:1) and U87 (14% E:T 30:1). The degree of cytolysis was observed in a titration dependent manner, with the percentage cytolysis values decreasing as the E:T ratio decreased. The percentage cytolysis value was calculated based on the maximum lysis by detergent. There was no cytolysis against K562 cells (negative control) and high cytolysis was seen against K562-OKT3 (positive control), by all CD8 cells indicating that all CD8 populations were capable of mounting an effective cytolysis response.

CNA1002 and CNA1004 expressing CD8+ CAR-T cells behaved similar to the mock transduced CD8+ cells, and did not show significant cytolysis against cancer cell lines MDA-MB-231 or U87, indicating that linker length plays a pivotal role in anti-dysfunctional P2X7 CAR-T killing and when optimized permits the targeting of a broad range of cancer types.

As can be seen in FIG. 12, the second functional assay confirmed the initial results and further demonstrated that CNA1003 expressing CD8+ CAR T cells showed specific lysis against the cancer cell lines M21 (approximately 25% E:T 30:1), OVACAR-3 (approximately 50% E:T 30:1), MDA-MB-231 (approximately 40% E:T 30:1) and U87 (approximately 28% E:T 30:1). In addition, CNA1002 expressing CD8+ CAR T cells demonstrated an ability to lyse OVCAR-3 (50% E: T 30:1) cell line.

Importantly, these results show that the CNA1003 CAR showed the broadest range of activity against the largest number of cancer cell lines. As such, it can be concluded that the anti-dysfunctional P2X7 CAR linker between 30 and 228 amino acids (specifically including 119 amino acids) provides efficacy against the largest number of cancer types compared to CARs having a linker domain of 12 amino acids (CNA1002) or 228 amino acids (CNA1004), with CNA1002 having activity against one cancer cell line and CNA1004 not having activity against any cancer cell lines.

### CD3+ CNA1003 CART Effector Function

### BrightGlo Luciferase Cytolytic Assay

Cancer cell lines stably expressing luciferase were purchased from CellBank Australia. Target cells (1×10⁴) were seeded (50 μl) into a round bottom 96-well plate, in triplicate for each condition tested. Additional control wells were plated for each target cell line. CNA 1003 CAR T cells were counted and serial dilutions were made. CAR T cells were added to the target cells in the following effector : target (E:T) ratios (30:1, 10:1, 3.3:1, 1.1:1). The 96-well plates were incubated for 16 h at 37° C. with 5% CO2. Subsequently, an equal volume of BrightGlo assay substrate (Promega) was added to each well, mixed well, incubated for 4 mins at room temperature, then a portion the mix was transferred to an opaque plate. The luminescence was read using a luminometer (GloMax Promega). The luminescence measured from leftover target cells was compared to the luminescence from target cells alone to calculate the percentage cytotoxicity of the CAR-T cells and mock transduced T cells.

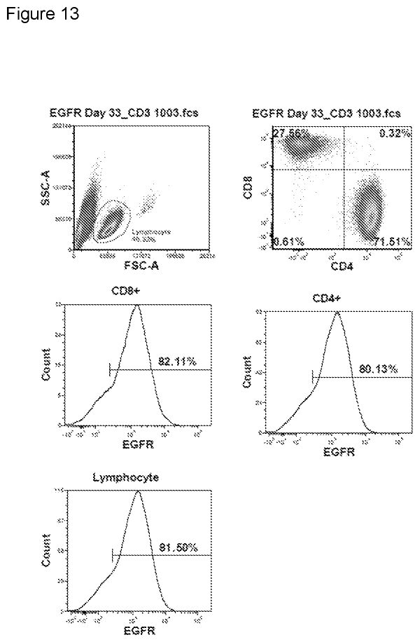

CD3+ T cells that expressed CNA1003 CAR were produced and expanded as described above. The CD3+ T cell population consisted of approximately 30% CD8+ T cells and 70% CD4+ T cells (FIG. 13). Surface expression of EGFRt was used as a proxy to measure transduction efficiency and approximately 80% of the CD3+ T cells were successfully transduced.

Cytotoxic function of the transduced CD3+ cells was assessed using the BrightGlo Luciferase assay system described above.

CAR T cells were co-cultured with target cancer cell lines at E:T ratios of 30:1, 10:1, 3.3:1 and 1.1:1 for 16 hr with the following cancer cell lines; PC3 (prostate cancer), C32 (melanoma), SkMel5 (melanoma), SkMel28 (melanoma), MDA-MB-231 (breast cancer), Be(2)M17 (neuroblastoma), Raji (lymphoma) and RD (rhabdomyosarcoma) and ASPC-1 (pancreatic cancer). CD3+ CAR T cells were used as effector cells and un-transduced CD3+ T cells were used as the negative control.

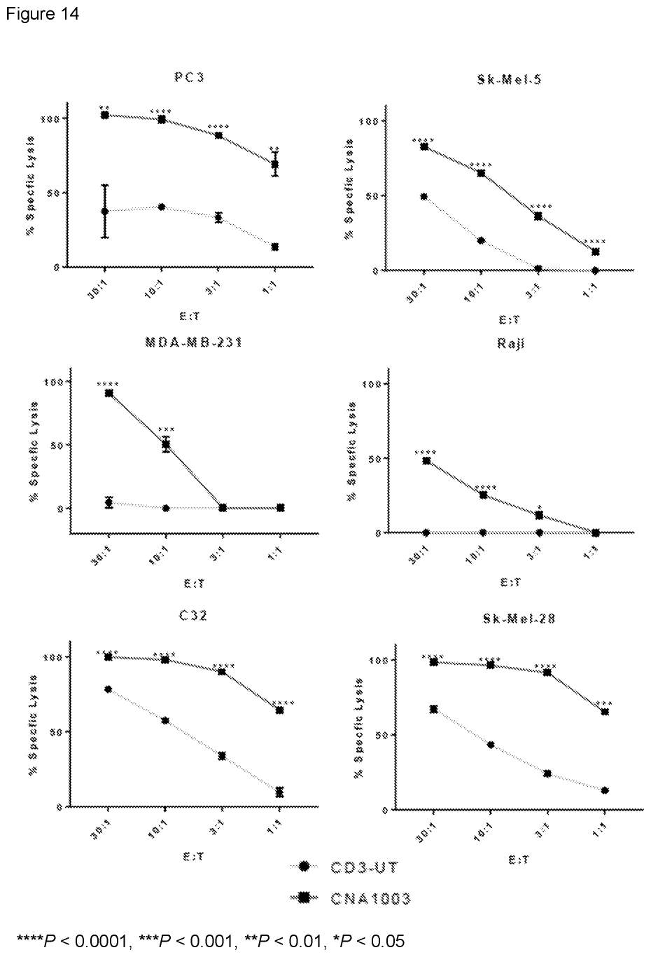

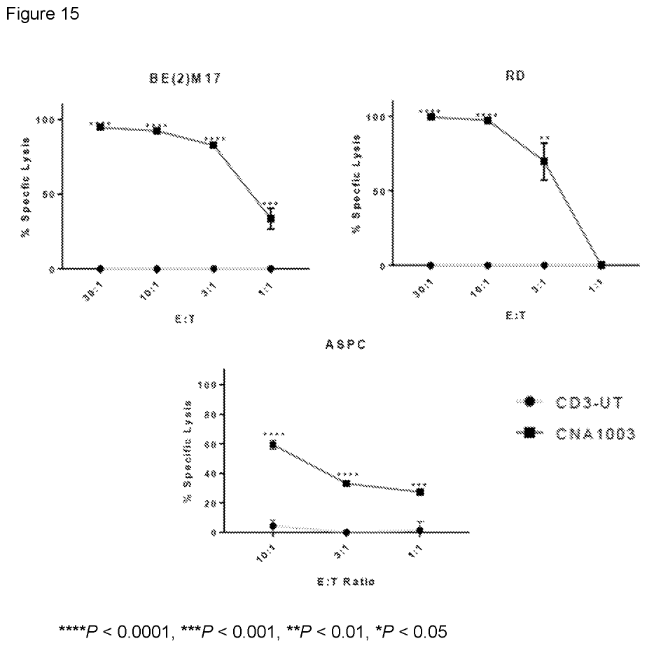

As shown in FIG. 14 and FIG. 15, specific cytolysis was observed by CD3+ CNA1003 CAR-T cells against all cancer cell lines tested; PC3 (100% E:T 30:1), C32 (100% E:T 30:1), SkMel5 (82% E:T 30:1), SkMel28 (98% E:T 30:1), MDA-MB-231 (90% E:T 30:1), Be(2)M17 (95% E:T 30:1), Raji (48% E:T 30:1), RD (99% E:T 30:1) and ASPc (59% E:T 30:1). A titration dependent effect was observed in some cancers, with the percentage cytolysis values decreasing as the E:T ratio decreased. Data was analysed unpaired Student's t-test comparing the CD3+ CNA1003 CAR T cells with CD3+ UT at each indicated time point. Data represent two independent experiments.

### CD8+ CNA1003 CART Effector Function

CD8+ T cells expressing CNA1003 CAR were generated and expanded as described above. Cytotoxic function of the CD8+ CNA1003 CAR T cells on the cancer cell lines (target cells) MDA-MB-231 (breast cancer), C32 (melanoma), PC3 (prostate cancer) and SKOV3 (ovarian cancer) was assessed using the BrightGlo Luciferase assay system described above. T cells were co-cultured with target cancer cell lines at E:T ratios of 30:1, 10:1, 3.3:1 and 1.1:1 for 16 hr. CD8+ CNA1003CAR cells were used as effector cells. Mock CD8 cells (un-transduced) were used as the control for non-specific killing.

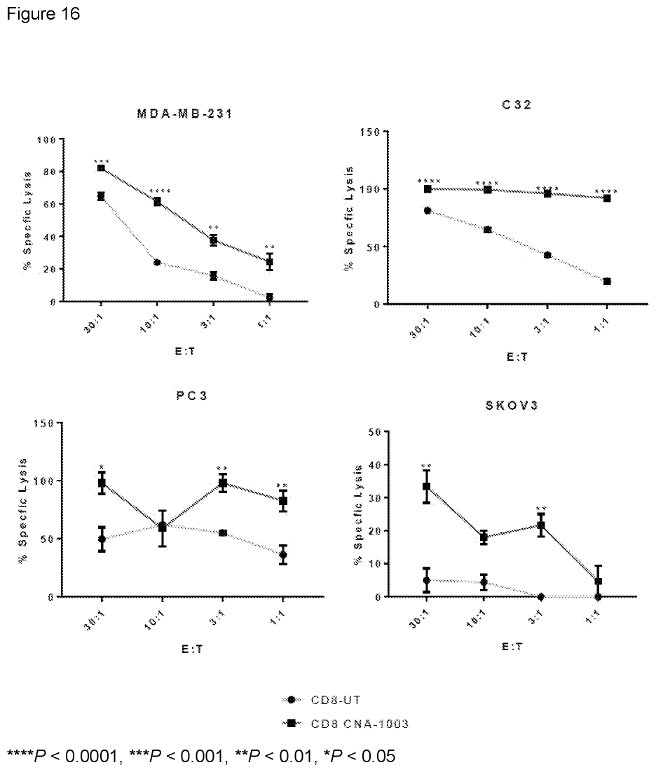

As can be seen in FIG. 16 specific cytolysis was observed against all four cancer cell lines tested, MDA-MB-231 (82% E:T 30:1), C32 (100% E:T 30:1), PC3 (98% E:T 30:1) and SKOV3 (33% E:T 30:1). A titration dependent effect was observed in some cancer cell lines, with the percentage cytolysis values decreasing as the E:T ratio decreased.

### CD4+ CNA1003 CART Effector Function

CD8+ T cells are the main cytotoxic T cell population. However, it is now known that that CD4+ T cells mediate potent anti-tumour activity.

CD4+ T cells expressing CNA1003 CAR were generated and expanded as described above. Cytotoxic function of the CD4+ CNA1003 CAR cells on the cancer cell lines (target cells) BT549 (breast cancer), OVCAR3 (ovarian cancer), C32 (melanoma) and PC3 (prostate cancer) was assessed using the BrightGlo Luciferase assay system described above. SKNDZ (neuroblastoma) cells were used as a negative control, based on prior data showing resistance to killing by CNA1003 expressing T cells.

CD4+ CNA1003 CAR-T cells were co-cultured with target cancer cell lines at E:T ratios of 30:1, 10:1, 3.3:1 and 1.1:1 for 16 h. Mock transduced (un-transduced—UT) CD4+ cells were used as the control for non-specific killing.

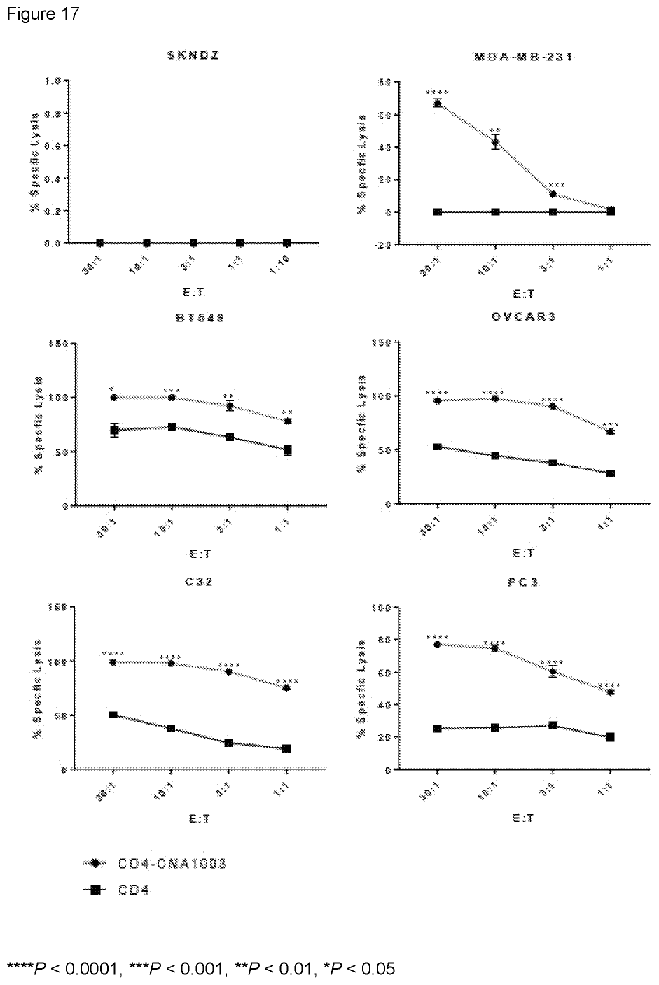

Specific cytolysis was observed against five cancer cell lines, MDA-MB-231 (67% E:T 30:1), BT549 (100% E:T 30:1), OVCAR3 (95% E:T 30:1), C32 (100% E:T 30:1) and PC3 (77% E:T 30:1) (FIG. 17). In all experiments a titration dependent effect was observed, with the percentage cytolysis values decreasing as the E:T ratio decreased. None of the CAR T cells showed cytolysis against the neuroblastoma cell line SKNDZ (negative control). Data was analysed unpaired Student's t test comparing the CNA1003 CAR T cells with CD4+ UT at the indicated time points. Data represent two independent experiments.

The above shows that CD4+ CAR T cells that recognises dysfunctional (specifically non-functional) P2X7 have significant cytotoxicity against a number of cancer cell lines that represent a broad range of cancer types.

### CD4+ CART T-Helper Function

The activation of CD4+ cells expressing the CNA family of CARs was measured by assaying for the cytokines IL-2, IFN-γ and TNF-α in a cytokine release assay set up in accordance with steps 1 and 2 (above). For the cytokine release assay, target cell lines were co-cultured with CD4+ mock or CD4+ cells expressing either the CN1002, CNA1003 or CNA1004 CARs for 24 h (5% CO₂) at 37° C. The concentration of cytokines; IL-2, IFN-γ and TNF-α in the supernatant was subsequently assayed using a Bio-Plex® validation kit.

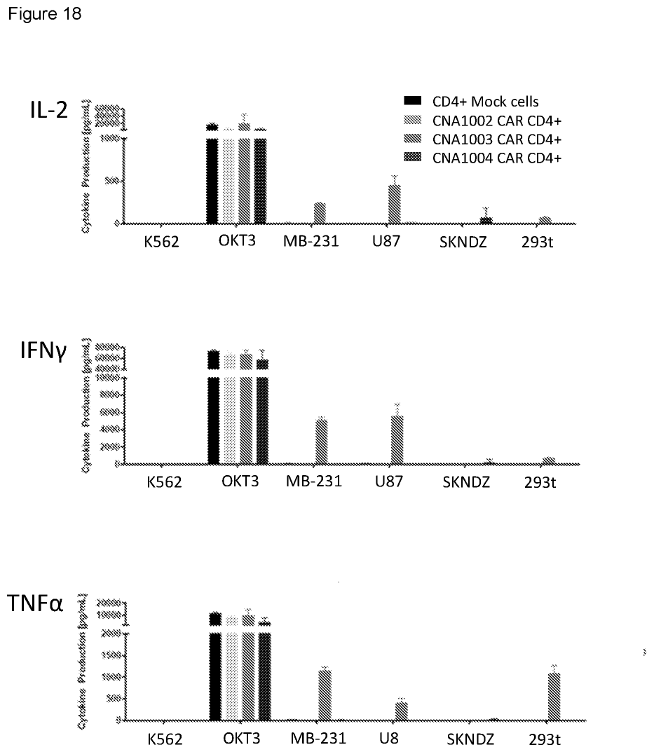

A first cytokine release assay (FIG. 18) was performed using K562 and 293T cell lines as negative controls, and K562 expressing OKT3 as a positive control. Three cancer cell lines: MDA-MB-231 (breast cancer), U87 (glioma) and SKNDZ (neuroblastoma) cell lines were used as target cells. CD4+ cells expressing CNA1002, CNA1003 or CNA1004 CARs were used as the effector cells. Mock CD4+ cells (un-transduced) were used as the negative control for effector cells.

As shown in FIG. 18, cell cultures containing effector cells expressing CNA1003 showed significant secretion of IL-2, IFN-γ and TNF-α when incubated against MB-231 (breast cancer cell) target cells and U87 (glioma) target cells. While CD4+ cells expressing CNA1002 and CNA1004 demonstrated little to no cytokine secretion when co-incubated with any of the target cells.

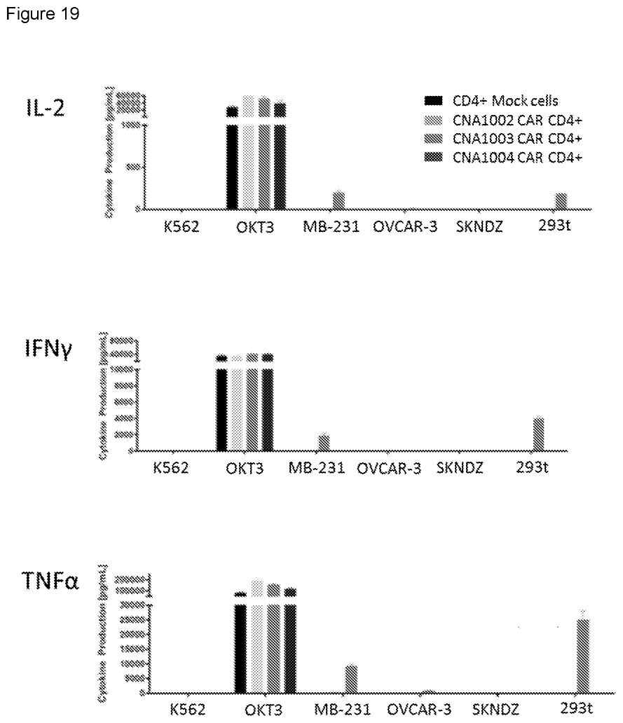

A second cytokine release assay (FIG. 19) was performed as described above, which included the above target cells (with the exception of U87 cells) and also included OVCAR3-ovarian cancer cells.

As can be seen in FIG. 19 elevated secretion of IL-2, IFN-γ and TNF-α was present in supernatants where CD4+ cells expressing CNA1003 were co-culture with the cancer cell line MDA-MB-231, confirming the results in the first cytokine release assay.

### EXAMPLE 3

### Protocol for Additional Chimeric Antigen Receptor

An exemplified protocol detailing the process of designing and expressing an anti-dysfunctional (specifically non-functional [nf]) P2X₇ receptor CAR according to an embodiment of the present invention is detailed as follows.

Design of PEP2-2-3 (anti-nf-P2x₇) Chimeric Antigen Receptor

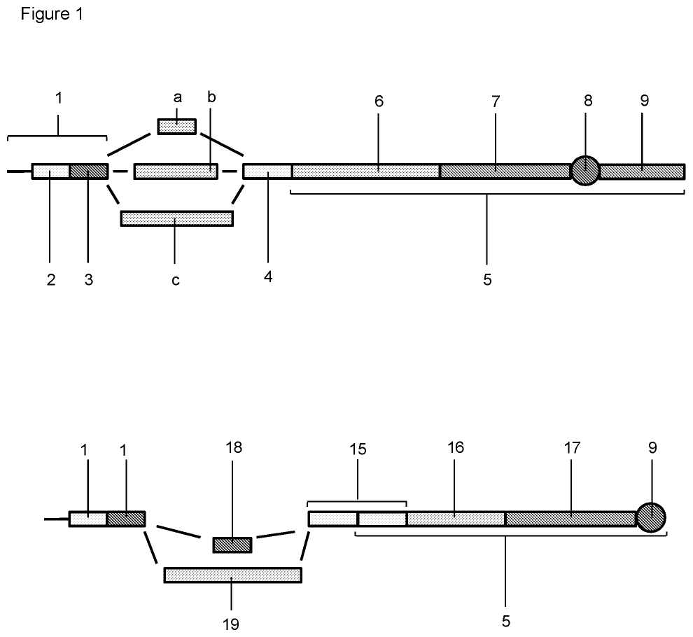

An anti-nf-P2x₇ chimeric antigen receptor (CAR) was designed according to the schematic illustrated in FIG. 1 (as described above) with hinge regions of either 30 amino acids (BLIV CAR short hinge linker; SEQ ID NO: 41) or 228 amino acids BLIV CAR long hinge; SEQ ID NO: 63).

### Lentival Vector Design and Assembly

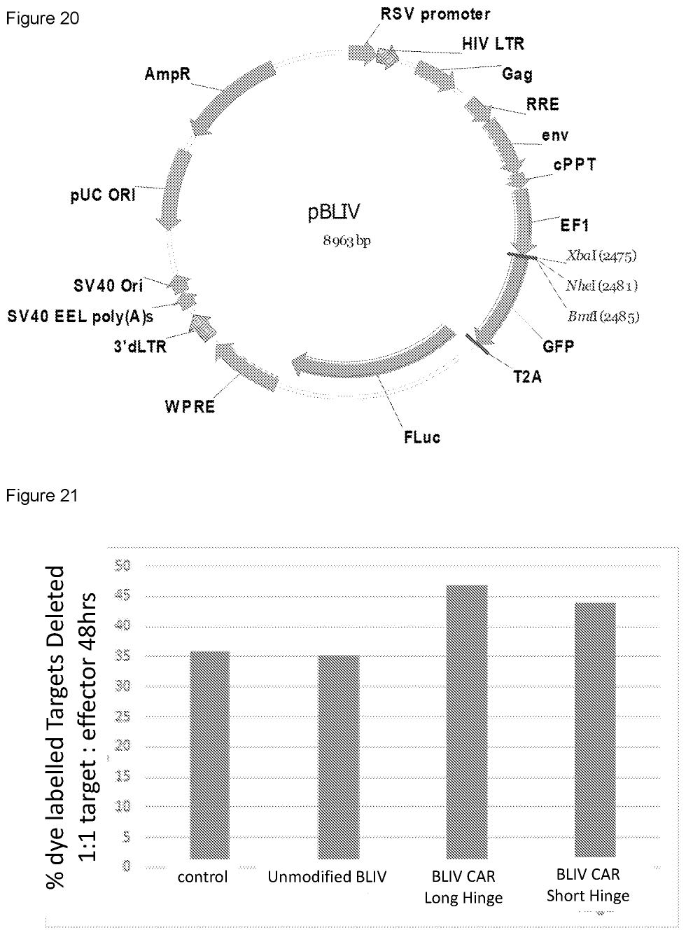

The designed CARs were incorporated into the BLIV lentiviral plasmid (System Biosciences, California, USA) illustrated in FIG. 20, which includes the fluorescence and bioluminescence reporting proteins, green-fluorescence protein (GFP) and Firefly Luciferase (FLuc). The BLIV plasmid further includes a T2A coding sequence between the GFP and FLuc reporter protein coding sequences permitting for post-translational separation of the FLuc and GFP proteins.

Sequences having homology to the sequences upstream and downstream of the NheI restriction site of the BLIV vector were added to the 5′ and 3′ ends of the designed CARs to result in the final nucleotide sequences set forth in SEQ ID NO 58 (CAR-short hinge) and SEQ ID NO: 59 (CAR-long hinge). The inclusion of the 5′ and 3′ sequences permitted incorporation of the anti-nf P2X₇ CAR into the BLIV vector using Gibson cloning.

The BLIV plasmid was restricted at the Nhel cloning site and the anti-nf P2X₇ CAR coding sequence was incorporated using Gibson assembly.

Cloning and E valuation of BLIV-CAR Vector

New England Biolabs 5-alpha Competent E. coli cells (provided in Gibson Assembly Cloning Kit) were transduced with the generated BLIV-CAR vectors as per the manufacturer's instructions.

Following incubation of the transduced (E.coli) cells, 10 colonies of bacteria transduced with BLIV-CAR-short hinge plasmid and 10 colonies of bacteria transduced with the BLIV-CAR-Long hinge plasmid were isolated, plasmid DNA was purified, and restricted with a BamHI restriction enzyme. The restricted DNA was analysed via gel electrophoresis for appropriate sized restriction fragments.

### Construction and Verification of Lentiviral Vectors

293T cells were used to package lentivirus from a 3 plasmid protocol according to the following method.

Day 1: 293T cells were seeded in 35 ml DMEM media with 10% serum in a T-225 flask such that the cells were substantially confluent the following day.

Day 2: 30 ug of one of the generated BLIV-CAR plasmids (or an unmodified BLIV plasmid), 30 ug of gag-pol plasmid delta 8.2, and 15 ug of VSV-G plasmid (pMD2.G), were added to OptiMEM media to a final volume of 750 ul, and mixed. 300 ul of PEI solution were added and incubated at room temperature for at least 20 minutes. The mixture was then added to the confluent 293T cells before incubation at 37° C.

Day 3: Supernatant was decanted from the 293T cells 24 hours after addition of the plasmid mixture and stored at 4° C. The decanted mixture was replaced with 35 ml of fresh media before further incubation at 37° C.

Day 4: 48 hours after addition of the plasmid mixture, the media was removed and combined with the supernatant from the 24 hour harvest. The combined supernatants were spun for 15 minutes at 1500 g to remove any remaining cellular debris. The supernatant was filtered through a 0.45 um filter, and then spun at 17,000 rpm for one hour. After centrifugation, the supernatant was decanted by hand, with 50 to 200 ul remaining in the tube. The centrifuge tube was placed in a 50 ml screw-top tube in order to prevent contamination and evaporation and the virus was allowed to resuspend at 4° C. overnight.

Day 5: The virus was resuspended off the bottom of the centrifuge tube and transferred into a new 1.5 ml tube. The resuspended virus was spun for 5 minutes in a microcentrifuge tube at 5000 rpm to remove any remaining debris.

Transfection of 293T cells with the BLIV-CAR-short hinge and BLIV-CAR-long hinge vector was assessed after 24 hours of incubation by the presence of GFP fluorescence. Supernatant collected at day 5 (as set out above) containing short- and long-hinge BLIV-CAR lentivirus vectors were incubated with fresh 293T cells and visualized for GFP fluorescence to test transduction capacity.

### CAR T Cell Effector Function

10⁸ CD8+ T cells were isolated from 50 ml of human blood using the RosetteSep™ human CD8+ T cell isolation kit (Stemcell technologies, Vancouver, Canada) in accordance with the manufacturer's instructions. Analysis of the purity, demonstrated that 76.6% of purified cells were CD8+

CD8+ T cells were incubated at 10⁵ cells per well with a 1:1 ratio of dynal T cell expander (CD3/CD28) beads. The CD8+ T cells were then incubated overnight together with lentiviral preparations, at a multiplicity of infection (MOI) of 5 or greater, containing either unmodified BLIV plasm ids, BLIV-CAR—short hinge plasm ids or BLIV-CAR-long hinge plasmids. Following incubation, the CD8+ T cells were washed before being co-cultured with the target cells.

Target cells expressing the non-functional P2X₇ receptor were provided by the mammary cancer cell line BT549 (ATCC HTB-122). These cells were dye-labelled using the fluorescent membrane intercalculating dye eFluor™ 670 (affymetrix eBioscience) as per the manufacturer's instructions.

Following dye labelling, target cells were co-culturing with the prepared CD8+ T cells at ratios of 10:1, 5:1, 1:1 and 0:1 (Car expressing T cells : targets).

After 24hrs of co-culture, cells were collected and analysed using Fluorescence-Activated Cell Sorting (FACS). The number of target cells containing the membrane intercalculating dye was quantified to assess if the co-cultured T cells led to target cell death or arrest of cell proliferation.

FIG. 21 illustrates that CD8+ T cells expressing the BLIV CAR short hinge of 30 amino acids demonstrated an increase in target cell lysis after 48 hrs, compared to co-culture of the target cells with non-transduced or control transduced (unmodified BLIV vector) CD8+ T cells. The efficacy of the 30 amino acid BLIV CAR short hinge was slightly less than that of the 228 amino acid BLIV CAR long hinge (SEQ ID NO:63) which showed slightly higher lysis than the BLIV-CAR short hinge.

### EXAMPLE 4

### Varying the Antigen-Recognition Domain

Six different CAR constructs comprising different antigen-recognition domains that bind the dysfunctional (specifically, non-functional (nf)) P2X7 receptors were compared for efficacy. Three CAR constructs comprised antigen-recognition domains (single domain antibodies or sdAb) consisting of peptide binders (CNA1003, CNA1103, CNA1203), two comprised antigen-recognition domains consisting of single variable chains (scfv) from a monoclonal antibody that recognises nfP2X7 (CNA1303 and CNA1403) and one was a di-peptide (CNA1503).

CNA1003, CNA1103 and CNA1203 were formed of the 3 CDRs from the variable heavy chain of antibodies specific for nfP2X7. CNA1303 and CNA1403 were formed of variable heavy chains from an antibody specific for nfP2X7 coupled, via an amino acid having the sequence set forth in SEQ ID NO: 69, to the variable light chains of distinct anti-nfP2X7 antibodies. CNA1503 was formed of two dAb region coupled by an amino acid having the sequence set forth in SEQ ID NO: 69.

These chimeric antigen receptor (CAR) construct consists of a human colony stimulating factor 2 receptor alpha (CSF2RA) leader sequence, one of the above described antigen-recognition domain (SEQ ID NO: 4—CNA1003; SEQ ID NO: 64—CNA1103; SEQ ID NO: 65—CNA1203; SEQ ID NO: 66—CNA1303; SEQ ID NO: 67—CNA1403; and SEQ ID NO: 68—CNA1503), a linker domain (IgG4 hinge-CH3—119 amino acids in length), CD28 transmembrane domain, intracellular signalling domains from 41BB and the CD3 zeta domain, with a terminal self-cleavage peptide T2A. A truncated form of the EGFR receptor (EGFRt), that lacks an intracellular signalling domain, was co-expressed from the same transcript via the self-cleavage peptide T2A. Surface expression of EGFRt was used as a proxy to measure transduction efficiency and purity of transduced T cells (as described above).

The CAR constructs were cloned into a lentiviral backbone, and CD8+ T cells were transduced to express CNA1003, CNA1103, CNA1203, CNA1303, CNA1403 and CNA1503, followed by expansion as described above.

The CD8+ CAR expressing T cells were used in a BrightGlo cytolysis assay following the protocol described above. Briefly, CAR expressing CD8+ T cells were co-cultured with target cancer cell lines at E:T ratios of 30:1, 10:1, 3.3:1 and 1.1:1 for 16 h. BrightGlo luciferase based assay system (Promega) was used to measure the cytolysis potential of the six CD8+ CAR T cell lines against four different cancer cell lines: MDA-MB-231 (breast cancer), C32 (melanoma), PC3 (prostate cancer) and SKOV3 (ovarian cancer). Mock CD8 cells (un-transduced—UT) were used as the control for non-specific killing.

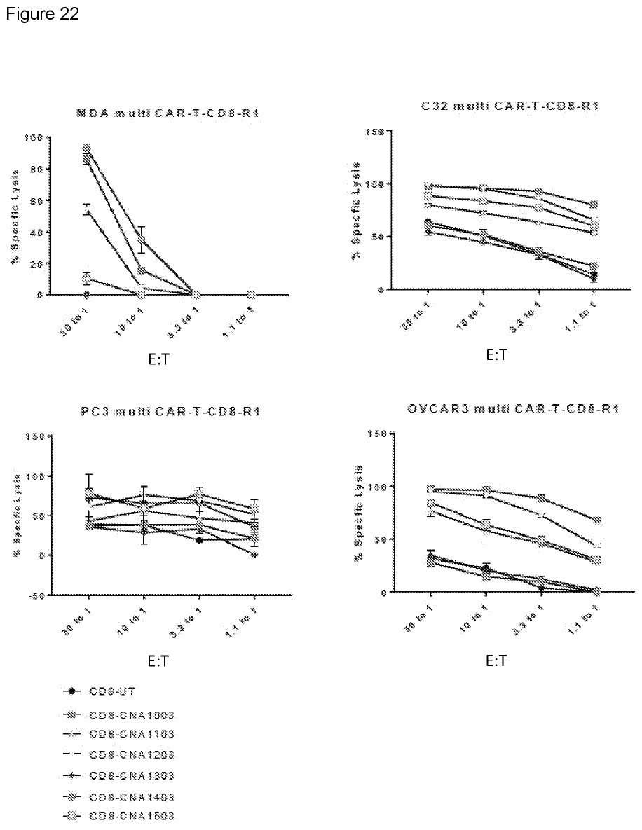

As can be seen in FIG. 22, CNA1003 CAR-T cells showed specific cytolysis against all four cancer cell lines, MDA-MB-231, C32, PC3 and OVCAR3. This was in line with our previous observations. CNA1203 and CNA1503 also showed specific cytolysis against MDA-MB-231, C32, PC3 and OVCAR3 cell lines. However, this was the same or slightly lower than CNA1003 CAR-T. CNA1403 showed specific cytolysis against the MDA-MB-231 cell line, but showed very low activity against the other three cell lines tested. CNA1103 showed specific cytolysis against C32 and OVCAR3 cell lines. In some cases a titration dependent effect was observed, with the percentage cytolysis values decreasing as the E:T ratio decreased.

These results demonstrate that while the antigen-recognition domain can influence the functionality of the CAR T cell, the anti-nf-P2X7 CAR T cells, having a linker length of 119 amino acids, maintain anti-cancer function against specific cell types. Further, the experiments set out herein permit a person skilled in the art to screen the anticancer function of CARs directed against nf-P2X7 against a variety of cancer types.

### EXAMPLE 5

### CNA1003 CAR T Cells Inhibit Tumour Growth of Prostate Cancer Cells In Vivo

To test the ability of CNA1003 human CD3+ T cells (CD4+ and CD8+ combined) and CD8+ T cells alone to inhibit tumour growth in vivo, a immunocompromised xenograft mouse model, implanted with tumour cell lines and CAR T cells, was used according to the following protocol.

5 to 8 week old male immunocompromised NOD.Cg-Prkdcscid Il2rgtm1Wjl/SzJ (NSG) mice were purchased from Animal Resource Centre (Perth, WA). Mice were housed in pathogen-free conditions with a 12 hour light/dark cycle. Mice were humanely euthanized by CO₂ asphyxiation prior to analysis.

### Xenograft Mouse Models

A prostate adenocarcinoma PC3 cell line engineered to express luciferase was maintained in Ham's F-12 Nutrient Mixture (Gibco) supplemented with 10% heat-inactivated foetal calf serum (FCS; Corning) and 100U/ml penicillin/streptomycin (Life Technologies) and was cultured at 37° C. in 5% CO₂. Cells were passaged every 2-3 days by rinsing the flasks with sterile PBS and dissociating cells with trypsin/EDTA in PBS (Gibco) for approximately 4 min at 37° C. Cells were regularly tested for mycoplasma and were confirmed to be mycoplasma free.

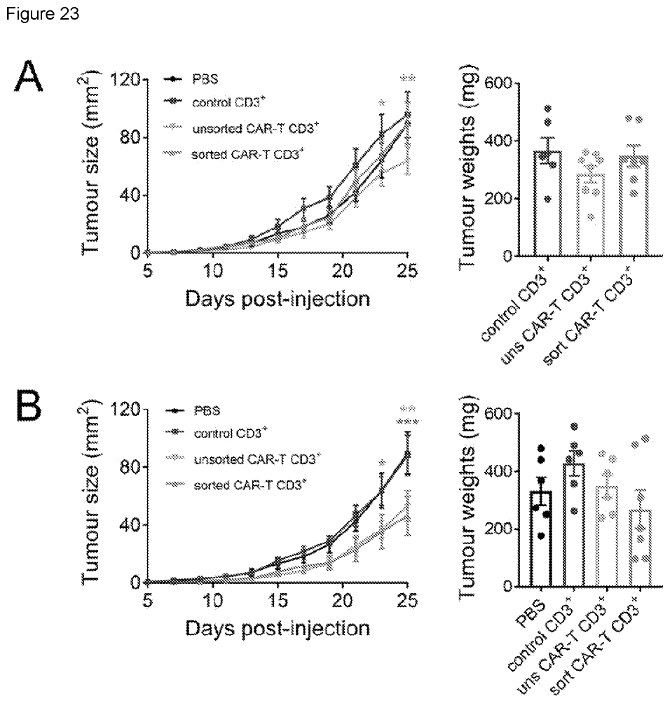

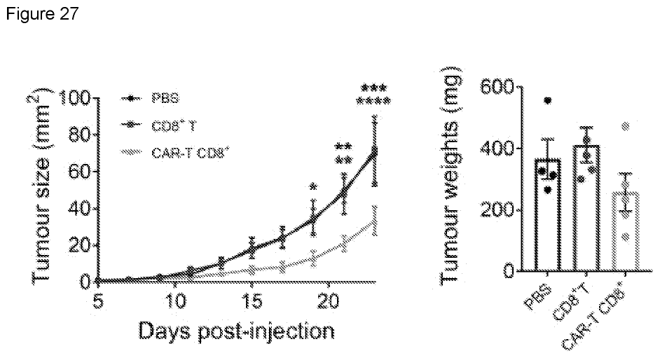

6 to 8 week old male NSG mice were injected subcutaneously into the right flank with 1×10⁶ PC3 human prostate cancer cells resuspended in sterile PBS. On d3 post-injection, 1×10⁷ human CAR T cell were administered. The administered CAR T cells were selected from one of the following groups: (i) CD3+ CNA1003 CAR T cells (including both CD4+ and CD8+ T cells); (ii) purified CD8+ CNA1003 CAR T cells; (iii) CD3+ CNA1003 CAR T cells sorted for EGFRt expression by flow cytometry (“sort CAR”—enriched for CAR expression); CD8+ CNA1003 CAR T cells sorted for EGFRt by flow cytometry (“sort CAR”), or un-transduced CD3+ T cells (FIGS. 23A—CD3+ and FIG. 27—CD8+) or as two doses with the second dose on d16 (FIGS. 23B—CD3+).

Cells were enriched (denoted “sort CAR”) by Fluorescence based cell sorting (FACS) following staining with a fluorescent conjugate labelled anti-EGFR antibody (EGFR Monoclonal Antibody (me1B3), eFluor 660) using protocols known in the art and as described above.

Tumours were measured every 2 days beginning on d5 using digital callipers by measuring the longest distance as length and the perpendicular distance as width. Tumour area was calculated as length×width. The health status of mice were monitored daily, and mice were euthanized when the length of the tumour was equal to or greater than 15 mm or when mice displayed disease symptoms including any combination of the following: ruffled coat, hunched posture, reluctance to move, laboured breathing, weight loss of 10% or more of initial weight and/or changes in behaviour or gait.

### Tissue Analysis

To analyse the infiltration and cytokine production of CAR T cells within tumours, the tumours were excised from mice, manually minced into small pieces and incubated in warm digest media for 1.5 hours with mixing every 15-20 min at 37° C. Digest media was prepared by supplementing DMEM (Gibco) with 5% heat-inactivated FCS (Corning), 2.5 mM CaCl₂, 10 mM HEPES (Gibco), 100U/ml penicillin/streptomycin (Life Technologies), 30 U/ml DNase I (Sigma-Aldrich) and 1 mg/ml collagenase IA (Sigma-Aldrich). Tumour homogenates were passed through a 70um filter (BD Biosciences) and incubated in mouse red cell lysis buffer for 5 min at 37° C.

Tumour infiltrating cells were analysed by flow cytometry. For staining cytokine expression, single cell suspensions of purified control or CAR T cells were incubated with warm IMDM supplemented with 10% FCS, 200 mM L-glutamine (Life Technologies), 100 U/ml penicillin/streptomycin (Life Technologies), 54 pM B-mercatopethanol (Sigma-Aldrich), 50 ng/ml phorbol-12-myristate 13-acetate (PMA; Sigma-Aldrich), 1 nM ionomycin (Life Technologies) and GolgiStop (at 1:1500 dilution; BD Biosciences) for 4 h at 37° C. Single cell suspensions were stained with near-infrared fixable dye and 10% human serum for 15 mins. Cells were then stained for 30 mins with α-hu CD8 BUV395 (RPA-T8) and CD4 BUV496 (SK3) antibodies. For intracellular staining, cells were incubated with Cytofix/Cytoperm for 20 mins, washed in Permwash buffer and stained with intracellular directly conjugated antibodies for 20 mins including IFNγ PE (B27), TNFα APC (MAb11), CD107a PECy7 (H4A3) and Granzyme B (Gzmb) BV421 (GB11) and Perforin (B-D48) T cell (Prf+) . All antibodies and staining reagents were purchased from BD Biosciences. After fixation in 1% paraformaldehyde, cells were acquired on the BD LSRFortessa X-20 flow cytometer. Data analysis was performed using FlowJo Software V.10 (Tree Star).

### Results

FIG. 23A and FIG. 27 demonstrate that a single dose of unsorted CD3+ CNA1003 CAR T cells (FIG. 23A) and CD8+ CNA1003 CAR T cells (FIG. 27) was effective at reducing tumour size and weight in a prostate cancer xenograft mouse model compared to mice treated with un-transduced T cells or PBS. FIG. 23B demonstrates that a double dose of sorted CD3+ T cells was more effective than a single dose of unsorted CD3+ T cells, while a double dose of unsorted CD3+ cells, with the second dose 13 days after the first, (FIG. 23B) was comparable to a single dose (FIG. 23A).

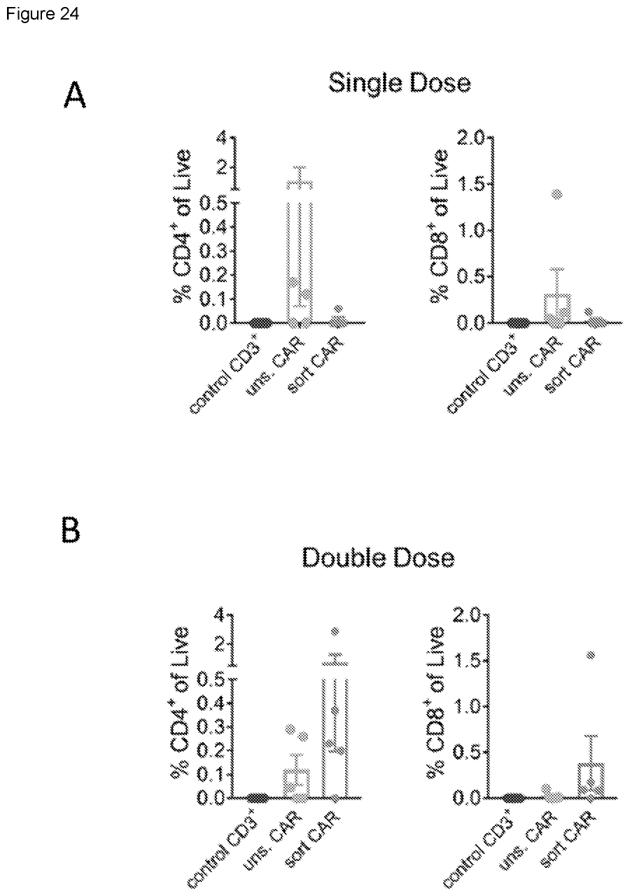

The infiltration of CD3+ CNA1003 CAR T cells into tumours was assessed together with the percentage of CD4+ T cells and CD8+ T cells (FIG. 24). Further, the secretion of cytokines: IFNγ and TNFα; markers of activation: Granzyme B (Gzmb+) and CD107a (LAMP-1); and markers of central memory T cells, CD45RA and CCR7 were assessed by flow cytometry.

FIG. 24 illustrates that both sorted and unsorted CAR T cells infiltrated tumours and were predominantly CD4+ T cells with lower levels of CD8+ cells.

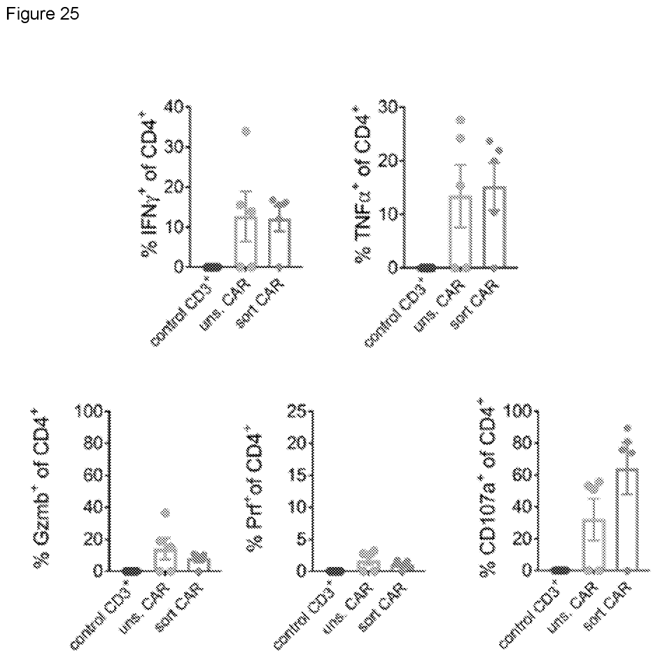

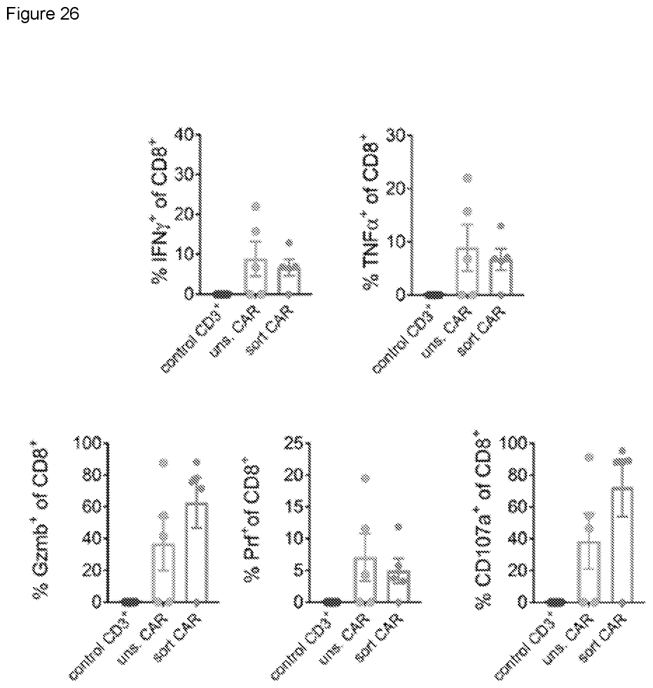

FIG. 25 and FIG. 26 illustrates that CD3+ CNA1003 CAR T cells (expressing both CD4+ and CD8+) infiltrate tumours and produce IFNγ and TNFα and are positive for Gzmb+, Prf+ and CD107a+ indicating that the infiltrating CAR T cells are activated in tumours. As expected, CD4+ (T helper cells) produce higher levels of IFNγ and TNFα while CD8+ cells show higher levels of Gzmb+ and Prf+.

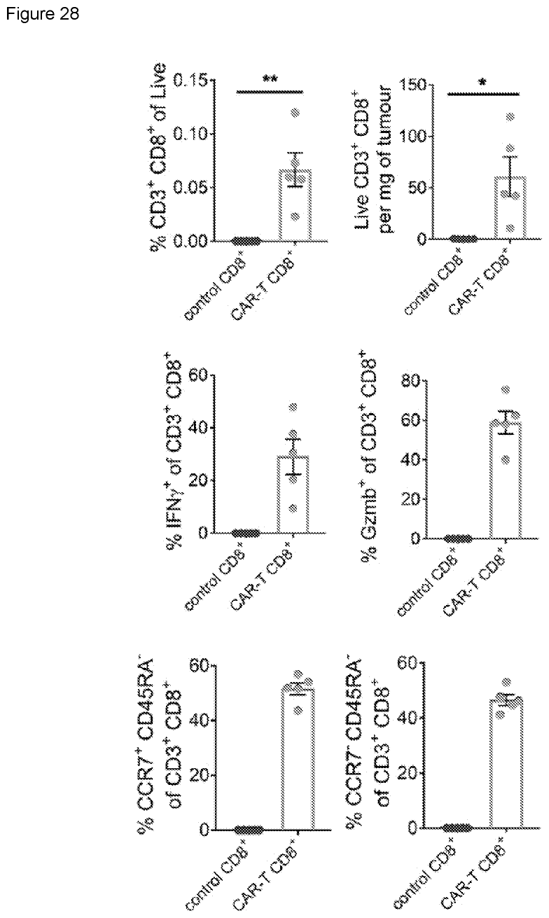

FIG. 27 shows that the administration of purified CD8+ CNA1003 CAR T cells (in the absence of substantial populations of CD4+ T cells) is effective at treating cancer and reducing tumour growth compared to mice treated with un-transduced CD8+ T cells or PBS. Further, FIG. 28 illustrates that substantial numbers of CD8+ T cells infiltrate tumours and are live and active as evidenced by the secretion of IFNγ and expression of Gzmb. Further, a substantial portion of the CD8+ infiltrates have a memory phenotype (CD45RA−) and express the lymphnode homing chemokine receptor CCR7 (approximately 50%).

In light of the above it is apparent that the delivery of CD3+ CNA1003 CAR T cells and CD8+ CNA1003 CAR T cells is able to treat cancer and inhibit tumour growth of PC3 human prostate cancer in NSG mice. This is evident when CD3+ CAR T cells are delivered as a bulk unsorted population in a single dose or as a sorted or unsorted population in two doses. Further, it has been shown that CNA1003+ CAR T cells are present within tumours at both d25 in mice receiving a single dose of cells and d27 in mice receiving two doses. This is in contrast to un-transduced CD3+ and CD8+ T cells which were not detectable at any of the endpoints. Further, a proportion of CAR T CD4+ expressing the cytokines IFNγ and TNFα and a significant proportion of CD8+ CAR T cells Granzyme B and CD107a which are all known to be important mediators of the killing activity of cytotoxic T lymphocytes. Further, a large portion of CD8+ CNA1003 CAR-T cells found in tumours have a central memory phenotype (CCR7+ and CD45RA−) and the approximately 45% having an effector T cell phenotype (CCR7− and CD45RA−) suggests the CD8+ CNA1003 CAR T cells in the tumours are equipped to both directly kill tumour cells as effector cells and also recirculate through the secondary lymphoid organs as central memory cells. This enables them to act as a self-renewing pool to maintain long-term protection after the initial dose.

### EXAMPLE 6

### Dose Response of CNA1003 CAR T Cells In Vivo

A prostate xenograft cancer cell model was used as set out in Example 5 above, with the exception of mice receiving 1×10⁷ CD3+ CNA1003 CAR T cells or 2×10⁷ CD3+ CNA1003 CART cells administered on day 3 (d3) post-tumour injection. In mice receiving a second dose, a further 1×10⁷ CD3+ CNA1003 CAR T cells were administered on d16 post-tumour injection. Un-transduced (UT) CD3+ T cells were used as controls at identical doses and administration regimens to the above.

Mice and tumour development was monitored as set out above in Example 5.

### Tissue Analysis and Flow Cytometry

Tumours were excised and single cell suspensions were obtained as set out in Example 5 above. For flow cytometry analysis, cells were then stained for 30 mins with the following fluorescently labelled antibodies: a-hu CD8 BUV395 (RPA-T8), CD4 BUV496 (SK3), CD45RA APC (HI100) and CCR7 PE (150503). For intracellular staining, cells were incubated with Cytofix/ Cytoperm for 20 mins, washed in Permwash buffer and stained with intracellular directly conjugated antibodies for 20 mins including Perforin (B-D48) and Granzyme B BV421 (GB11). All antibodies and staining reagents were purchased from BD Biosciences. After fixation in 1% paraformaldehyde, cells were analysed on the BD LSRFortessa X-20 flow cytometer. Data analysis was performed using FlowJo Software V.10 (Tree Star).

### Results

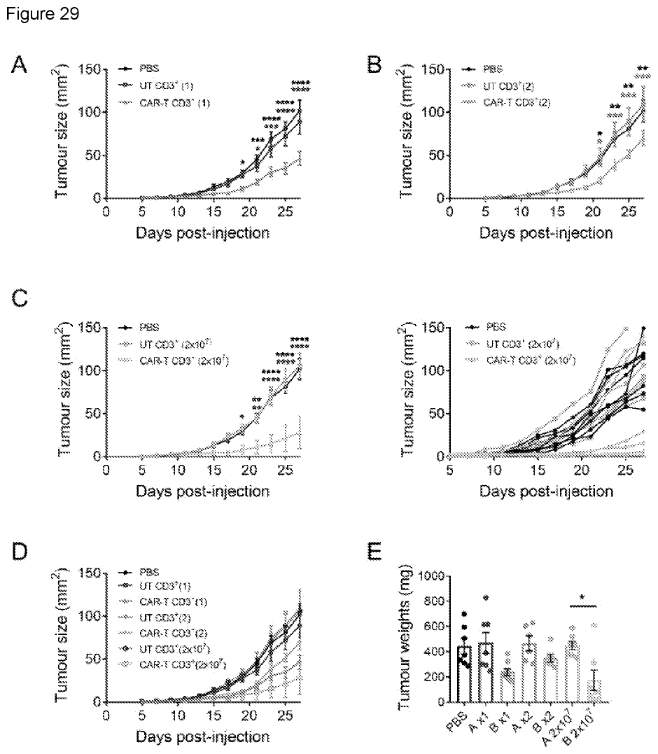

FIGS. 29A and 29B illustrate that a single dose (FIG. 29A) or a double dose (FIG. 29B) of 1×10⁷ CD3+ CAR-T was effective at reducing tumour size in the prostate cancer xenograft mouse model compared to mice treated with un-transduced (UT) T cells or PBS. Further, FIG. 29C illustrates that a single dose of 2×10⁷ CD3+ CNA1003 CAR T cells resulted in a further decrease in tumour size compared to a dose of 1×10⁷ cells. Analysis of individual mice (FIG. 29C right hand panel) showed a marked decrease in tumour growth in 6 out of 7 mice treated compared to PBS treated mice or mice administered un-transduced (UT) CD3+ T cells. Notably, 4 of the 7 mice showed an almost complete absence of tumours.

FIGS. 29D and 29E illustrate the tumour growth in mice administered a single dose of 1×10⁷ CD3+ CNA1003 CAR T cells (CAR-T CD3+ (1)), two doses of 1×10⁷ CD3+ CNA1003 CAR T cells (CAR-T CD3+ (2)) 13 days apart, or a single dose of 2×10⁷ CD3+ CNA1003 CAR T cells (CAR-T CD3+ (2×10⁷)). As can be seen in FIG. 29D mice administered a single dose of 2×10⁷ CD3+ CNA1003 CART cells on d3 had lower tumour size compared to all other groups receiving CD3+ CNA1003 CAR T cells. Further all CAR treatments groups had a decrease in tumour size compared to mice treated with PBS or un-transduced CD3+ T cells. These results were reflected when tumour weight was analysed with each CAR treatment group (denoted by the letter “B”) having lower tumour weights compared to the un-transduced groups (denoted by the letter “A”) or the PBS treated group (FIG. 29E).

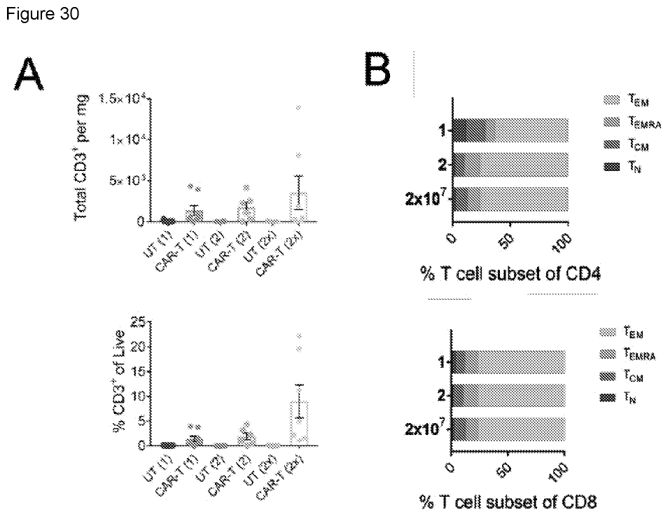

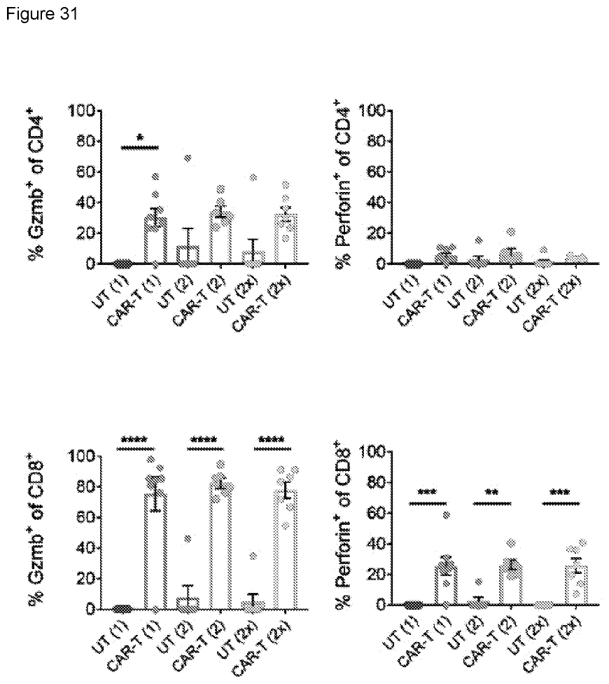

The phenotype of tumour infiltrating T cells were analysed and the results are presented in FIG. 30A and 30B. FIG. 30A illustrates the percentage of live CD3+ cells per mg of tissue. As can be seen, tumours from mice administered with a single dose of 2×10⁷ CD3+ CNA1003 CAR T cells (CAR T 2x) had the largest percentage of live CD3+ T cells, with all treatment groups (CAR-T (1)—1 dose of 1×10⁷ CAR cells; CART (2)—2 doses of 1×10⁷ CAR cells; and CAR-T (2x)—1 dose of 2×10⁷ CAR cells) having tumour infiltrating CD3+ T cells, while un-transduced (UT) controls had negligible tumour infiltration of T cells.

Analysis of the composition of the tumour infiltrating cell population is presented in FIG. 30B. As can be seen, the majority of CD4+ and CD8+ T cells present in tumours from mice administered CD3+ CNA1003 CAR T cells had an effector phenotype (T_{EM}−CDR7−CD45RA−). There was also a substantial proportion of central memory T cells (T_{CM}—CDR7+CD45RA−) and terminally differentiated effector memory cells (T_{EMRA}—CDR7+CD45RA+) with both of these cell populations being antigen experienced cells which can migrate to the lymph nodes. A small population of naïve T cells (T_{N}—CDR7−CD45RA+) is also present.

FIG. 31 illustrates the granzyme b (Gzmb+) and Perforin expression of CD4+ and CD8+ T cells. Together Granzyme b and Perforin constitute the major killing mechanism of cytotoxic T lymphocytes indicating that the tumour infiltrating CAR T cells are cytotoxic.

### EXAMPLE 7

### CNA1003 CAR T Cells Inhibit Tumour Growth of Breast Cancer Cells In Vivo

6 week old female immunocompromised NSG mice were purchased from Animal Resource Centre (Perth, Wash.). Mice were housed in pathogen-free conditions with a 12 hour light/dark cycle. Mice were humanely euthanized by CO₂ asphyxiation.

### Xenograft Mouse Models

For the primary tumour model, 12 to 13 week female NSG mice were injected subcutaneously into the 4th left mammary fat pad (L4) with 2×10⁶ MDA-MB-231 human breast cancer cells resuspended in sterile PBS:Matrigel such that the final protein concentration was 4 to 6mg/mL.

On d3 post-injection, 1×10⁷ CD8+ CNA1003 CAR-T cells or control CD8+ T cells (purified from human blood and transduced with lentivirus) were injected intravenously. Tumours were measured in every 2 days starting on d5 using digital callipers, measuring the longest distance as length and the perpendicular distance as width. Tumour area was calculated as length×width. The health status of mice were monitored daily and euthanized when the tumour length was equal to or greater than 15 mm or when mice showed a ruffled coat, hunched posture, reluctance to move or laboured breathing, weight loss of 10% of more of initial weight and/or changes in behaviour or gait.

Lung Metastatic Nodule Visualization

Mice were euthanized by CO₂ asphyxiation, the ribcage was dislodged and the trachea exposed. 15% black ink (Parker) resuspended in water was injected intracheally using a 26 gauge needle until lungs were filled completely. Lungs were removed and immediately destained in 55% EtOH, 6% formaldehyde, 8% glacial acetic acid resuspended in water (Fekete's solution). Lungs were separated into 5 lobes and white nodules were counted.

### Results

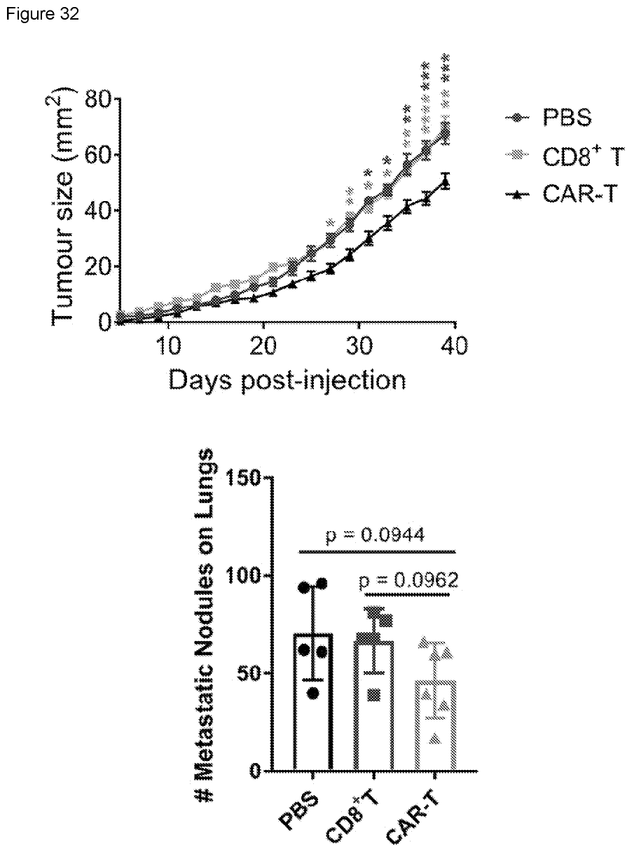

FIG. 32 shows that the administration of purified CD8+ CNA1003 CAR T (“CAR-T”) cells are effective at (i) treating cancer and reducing tumour growth (size and weight) in a breast cancer xenograft mouse model compared to mice treated with un-transduced CD8+ T cells (“CD8+ T”) or PBS, and (ii) reducing metastasis formation in a secondary site such as the lungs.

In light of the above, it is apparent that administration of CD8+ CNA1003 CAR T cells can inhibit tumour growth of human breast cancer in NSG mice compared to mice given control CD8+ human T cells or PBS. This is associated with fewer nodules on the lungs which arise from the spontaneous metastasis of the highly metastatic MDA-MB-231 tumours in mice receiving CD8+ CNA1003 CART cells.

All methods described herein can be performed in any suitable order unless indicated otherwise herein or clearly contradicted by context. The use of any and all examples, or exemplary language (e.g., “such as”, “i.e.”) provided herein, is intended merely to better illuminate the example embodiments and does not pose a limitation on the scope of the claimed invention unless otherwise claimed. No language in the specification should be construed as indicating any non-claimed element as essential.

The description provided herein is in relation to several embodiments which may share common characteristics and features. It is to be understood that one or more features of one embodiment may be combinable with one or more features of the other embodiments. In addition, a single feature or combination of features of the embodiments may constitute additional embodiments.

The subject headings used herein are included only for the ease of reference of the reader and should not be used to limit the subject matter found throughout the disclosure or the claims. The subject headings should not be used in construing the scope of the claims or the claim limitations.

Those skilled in the art will appreciate that the invention described herein is susceptible to variations and modifications other than those specifically described. It is to be understood that the invention includes all such variations and modifications. The invention also includes all of the steps, features, compositions and compounds referred to, or indicated in this specification, individually or collectively, and any and all combinations of any two or more of the steps or features.

Also, it is to be noted that, as used herein, the singular forms “a”, “an” and “the” include plural aspects unless the context already dictates otherwise.

1. A chimeric antigen receptor including an antigen-recognition domain recognising a dysfunctional P2X₇ receptor, a transmembrane domain and a linker domain, wherein the linker domain consists of between 12 to 228 amino acids.

2. The chimeric antigen receptor according to claim 1, where in the linker domain consists of between 30 to 228 amino acids, between 50 to 200 amino acids, between 70 to 180 amino acids, between 90 to 160 amino acids, between 110 to 130 amino acids, between 115 to 125 amino acids, between 117 to 121 amino acids, or about 119 amino acids. 3-9 (Canceled)

10. The chimeric antigen receptor according to claim 1, wherein the linker domain comprises an amino acid sequence homologous to an immunoglobulin hinge region.

11. The chimeric antigen receptor according to claim 1, wherein the linker domain comprises an amino acid sequence that is at least 50%, 60%, 70%, 80%, 90%, 93%, 96% or 99% identical to an immunoglobulin hinge region of IgG, IgD, IgA, or the Constant Heavy (CH) 2 region of IgM or IgE, or a function variant thereof having at least 50%, 60%, 70%, 80%, 90%, 93%, 96% or 99% sequence identity.

12. The chimeric antigen receptor according to claim 1, wherein the linker domain comprises an amino acid sequence that is at least 50%, 60%, 70%, 80%, 90%, 93%, 96% or 99% identical to a hinge region from an IgG isotype immunoglobulin, or a functional variant thereof having at least 50%, 60%, 70%, 80%, 90%, 93% or 96% sequence identity.

13. The chimeric antigen receptor according to claim 1, wherein the linker domain comprises an amino acid sequence homologous that is at least 50%, 60%, 70%, 80%, 90%, 93%, 96% or 99% identical to the hinge region of the IgG1, IgG2 or IgG4 subclass of immunoglobulin or a functional variant thereof having at least 50%, 60%, 70%, 80%, 90% or 93% sequence identity.

14. The chimeric antigen receptor according to claim 1, wherein the linker domain comprises an amino acid sequence homologous to a hinge region from an IgG isotype immunoglobulin including a CXXC motif.

15. The chimeric antigen receptor according to claim 1, wherein the linker domain comprises an amino acid sequence homologous to a CXXC motif that is selected from the group of CPPC, CPRC or CPSC

16. The chimeric antigen receptor according to claim 1, wherein the linker domain comprises one or more amino acid sequences homologous that is at least 50%, 60%, 70%, 80%, 90%, 93%, 96% or 99% identical to:
(a) a constant heavy (CH) region of an immunoglobulin;
(b) a CH1 region, a CH2 region, a CH3 region and/or a CH4 region of an immunoglobulin; or
(c) a CH2 region and/or a CH3 region of an IgG isotype immunoglobulin.

17-18. (canceled)

19. The chimeric antigen receptor according to claim 1, wherein the linker domain consists of:
(a) one or more immunoglobulin hinge region(s) and/or one or more CH region(s) of an immunoglobulin;
(b) an IgG hinge region and one or more CH region(s) of an immunoglobulin; or
(c) an IgG hinge region and/or a CH2 or CH3 region of an immunoglobulin.

20-21. (canceled)

22. The chimeric antigen receptor according to claim 1, wherein the linker domain comprises an amino acid sequence according to any one of SEQ ID NOs: 9 to 17, or comprises a sequence at least 50%, 60%, 70%, 80%, 90%, 93% or 96% identical to any one of SEQ ID Nos: 9 to 17.

23. (canceled)

24. The chimeric antigen receptor according to claim 1, wherein the chimeric antigen receptor does not comprise an amino acid sequence in the linker domain that substantially binds to an Fc receptor.

25-27. (canceled)

28. The chimeric antigen receptor according to claim 1, wherein the dysfunctional P2X₇ receptor has a reduced capacity to bind ATP compared to an ATP-binding capacity of a fully functional P2X₇ receptor.

29. The chimeric antigen receptor according to claim 1, wherein the dysfunctional P2X₇ receptor has a conformational change that renders the receptor dysfunctional.

30. (canceled)

31. The chimeric antigen receptor according to claim 1, wherein the dysfunctional P2X₇ receptor has a conformational change that comprises an amino acid that has changed from a trans-conformation to a cis-conformation is proline at amino acid position 210 of the dysfunctional P2X₇ receptor.

32-34. (canceled)

35. The chimeric antigen receptor according to claim 1, wherein the transmembrane domain comprises all or part of the transmembrane domain of CD8 or CD28.

36-37. (canceled)

38. A genetically modified cell, the cell comprising the chimeric antigen receptor according to claim 1 or comprising a nucleic acid encoding the chimeric antigen receptor according to claim 1.

39-51. (canceled)

52. A method of killing a cell expressing a dysfunctional P2X₇ receptor, the method comprising exposing the cell expressing a dysfunctional P2X₇ receptor to a cell expressing a chimeric antigen receptor according to claim 1.

53. The method according to claim 52, wherein the cell expressing a dysfunctional P2X₇ receptor is a cancer cell.

54-58. (canceled)

59. A pharmaceutical composition comprising a genetically modified cell comprising a chimeric antigen receptor according to claim 1, and a pharmaceutically acceptable carrier or excipient.

60-64. (canceled)


SEQUENCE LISTING (69 sequences)

SEQ ID NO: 1 - DNA, 3680 bp, Homo sapiens
gtcattggaggagcttgaagttaaagactcctgctaaaaaccagtacgtttcattttgcagttactgggagggggcttgctgtggccctgtcaggaagagtagagctctggtccagctccgcgcagggagggaggctgtcaccatgccggcctgctgcagctgcagtgatgttttccagtatgagacgaacaaagtcactcggatccagagcatgaattatggcaccattaagtggttcttccacgtgatcatcttttcctacgtttgctttgctctggtgagtgacaagctgtaccagcggaaagagcctgtcatcagttctgtgcacaccaaggtgaaggggatagcagaggtgaaagaggagatcgtggagaatggagtgaagaagttggtgcacagtgtctttgacaccgcagactacaccttccctttgcaggggaactctttcttcgtgatgacaaactttctcaaaacagaaggccaagagcagcggttgtgtcccgagtatcccacccgcaggacgctctgttcctctgaccgaggttgtaaaaagggatggatggacccgcagagcaaaggaattcagaccggaaggtgtgtagtgtatgaagggaaccagaagacctgtgaagtctctgcctggtgccccatcgaggcagtggaagaggccccccggcctgctctcttgaacagtgccgaaaacttcactgtgctcatcaagaacaatatcgacttccccggccacaactacaccacgagaaacatcctgccaggtttaaacatcacttgtaccttccacaagactcagaatccacagtgtcccattttccgactaggagacatcttccgagaaacaggcgataatttttcagatgtggcaattcagggcggaataatgggcattgagatctactgggactgcaacctagaccgttggttccatcactgccgtcccaaatacagtttccgtcgccttgacgacaagaccaccaacgtgtccttgtaccctggctacaacttcagatacgccaagtactacaaggaaaacaatgttgagaaacggactctgataaaagtcttcgggatccgttttgacatcctggtttttggcaccggaggaaaatttgacattatccagctggttgtgtacatcggctcaaccctctcctacttcggtctggccgctgtgttcatcgacttcctcatcgacacttactccagtaactgctgtcgctcccatatttatccctggtgcaagtgctgtcagccctgtgtggtcaacgaatactactacaggaagaagtgcgagtccattgtggagccaaagccgacattaaagtatgtgtcctttgtggatgaatcccacattaggatggtgaaccagcagctactagggagaagtctgcaagatgtcaagggccaagaagtcccaagacctgcgatggacttcacagatttgtccaggctgcccctggccctccatgacacacccccgattcctggacaaccagaggagatacagctgcttagaaaggaggcgactcctagatccagggatagccccgtctggtgccagtgtggaagctgcctcccatctcaactccctgagagccacaggtgcctggaggagctgtgctgccggaaaaagccgggggcctgcatcaccacctcagagctgttcaggaagctggtcctgtccagacacgtcctgcagttcctcctgctctaccaggagcccttgctggcgctggatgtggattccaccaacagccggctgcggcactgtgcctacaggtgctacgccacctggcgcttcggctcccaggacatggctgactttgccatcctgcccagctgctgccgctggaggatccggaaagagtttccgaagagtgaagggcagtacagtggcttcaagagtccttactgaagccaggcaccgtggctcacgtctgtaatcccagcgctttgggaggccgaggcaggcagatcacctgaggtcgggagttggagacccgcctggctaacaaggcgaaatcctgtctgtactaaaaatacaaaaatcagccagacatggtggcatgcacctgcaatcccagctactcgggaggctgaggcacaagaatcacttgaacccgggaggcagaggttgtagtgagcccagattgtgccactgctctccagcctgggaggcacagcaaactgtcccccaaaaaaaaaaaagagtccttaccaatagcaggggctgcagtagccatgttaacatgacatttaccagcaacttgaacttcacctgcaaagctctgtggccacattttcagccaaagggaaatatgctttcatcttctgttgctctctgtgtctgagagcaaagtgacctggttaaacaaaccagaatccctctacatggactcagagaaaagagattgagatgtaagtctcaactctgtccccaggaagttgtgtgaccctaggcctctcacctctgtgcctctgtctccttgttgcccaactactatctcagagatattgtgaggacaaattgagacagtgcacatgaactgtcttttaatgtgtaaagatctacatgaatgcaaaacatttcattatgaggtcagactaggataatgtccaactaaaaacaaacccttttcatcctggctggagaatgtggagaactaaaggtggccacaaattctttgacactcaagtcccccaagacctaagggttttatctcctccccttgaatatgggtggctctgattgctttatccaaaagtggaagtgacattgtgtcagtttcagatcctgatcttaagaggctgacagcttctacttgctgtcccttggaactcttgctatcggggaagccagacgccatttaaaagtctgcctatcctggccaggtgtggtggctcacacctgtaatcccagcactttgggagaccaaggcgggcggatcacttaaagtcaggagtccaagaccagactcgccaacatggtgaaaccgtatctctaataaaaatacaaaaattagctgggcatggtgcgggcacctgtagtcctagctatcaagaggctgagacaggagaaacacttgaacctgggaggtggaggttgcattgagctgagatcgtgccactgcactccaggctgggtgacagagcgagactccatctcaaaaaaaaaaaaaagaaaaaaaaaatgtctgcctatcctgagactgccctgctgtgaggaagcccaagcagtcacgtggacagtgcctgaccagccccagctttcaagccatccaagcccagtcaccaaacatgagagagaagaagccttcaggtgattctggactccactaacatatgactgataccgcatgatacatcccaagtgagaactgccccataaatccagaaaaccacattgctatcttaagtccctaagtttggggcttatttgttccacagcaacaggtaactggaacagagggcaagcctgatgaatgggcacacagactcagcccataccttccctggttctaatgttctcagggagcccggaccaaccctgggagcctcaggaacttaggtttccactggacagttctagaagggctatagaccaaatcaggtaactcaccagaccagccttggaatctatcaaatctaactgctgagctaccca

SEQ ID NO: 2 - DNA, 1788 bp, Homo sapiens
atgccggcctgctgcagctgcagtgatgttttccagtatgagacgaacaaagtcactcggatccagagcatgaattatggcaccattaagtggttcttccacgtgatcatcttttcctacgtttgctttgctctggtgagtgacaagctgtaccagcggaaagagcctgtcatcagttctgtgcacaccaaggtgaaggggatagcagaggtgaaagaggagatcgtggagaatggagtgaagaagttggtgcacagtgtctttgacaccgcagactacaccttccctttgcaggggaactctttcttcgtgatgacaaactttctcaaaacagaaggccaagagcagcggttgtgtcccgagtatcccacccgcaggacgctctgttcctctgaccgaggttgtaaaaagggatggatggacccgcagagcaaaggaattcagaccggaaggtgtgtagtgtatgaagggaaccagaagacctgtgaagtctctgcctggtgccccatcgaggcagtggaagaggccccccggcctgctctcttgaacagtgccgaaaacttcactgtgctcatcaagaacaatatcgacttccccggccacaactacaccacgagaaacatcctgccaggtttaaacatcacttgtaccttccacaagactcagaatccacagtgtcccattttccgactaggagacatcttccgagaaacaggcgataatttttcagatgtggcaattcagggcggaataatgggcattgagatctactgggactgcaacctagaccgttggttccatcactgccgtcccaaatacagtttccgtcgccttgacgacaagaccaccaacgtgtccttgtaccctggctacaacttcagatacgccaagtactacaaggaaaacaatgttgagaaacggactctgataaaagtcttcgggatccgttttgacatcctggtttttggcaccggaggaaaatttgacattatccagctggttgtgtacatcggctcaaccctctcctacttcggtctggccgctgtgttcatcgacttcctcatcgacacttactccagtaactgctgtcgctcccatatttatccctggtgcaagtgctgtcagccctgtgtggtcaacgaatactactacaggaagaagtgcgagtccattgtggagccaaagccgacattaaagtatgtgtcctttgtggatgaatcccacattaggatggtgaaccagcagctactagggagaagtctgcaagatgtcaagggccaagaagtcccaagacctgcgatggacttcacagatttgtccaggctgcccctggccctccatgacacacccccgattcctggacaaccagaggagatacagctgcttagaaaggaggcgactcctagatccagggatagccccgtctggtgccagtgtggaagctgcctcccatctcaactccctgagagccacaggtgcctggaggagctgtgctgccggaaaaagccgggggcctgcatcaccacctcagagctgttcaggaagctggtcctgtccagacacgtcctgcagttcctcctgctctaccaggagcccttgctggcgctggatgtggattccaccaacagccggctgcggcactgtgcctacaggtgctacgccacctggcgcttcggctcccaggacatggctgactttgccatcctgcccagctgctgccgctggaggatccggaaagagtttccgaagagtgaagggcagtacagtggcttcaagagtccttactga

SEQ ID NO: 3 - protein, 595 aa, Homo sapiens
MPACCSCSDVFQYETNKVTRIQSMNYGTIKWFFHVIIFSYVCFALVSDKLYQRKEPVISSVHTKVKGIAEVKEEIVENGVKKLVHSVFDTADYTFPLQGNSFFVMTNFLKTEGQEQRLCPEYPTRRTLCSSDRGCKKGWMDPQSKGIQTGRCVVYEGNQKTCEVSAWCPIEAVEEAPRPALLNSAENFTVLIKNNIDFPGHNYTTRNILPGLNITCTFHKTQNPQCPIFRLGDIFRETGDNFSDVAIQGGIMGIEIYWDCNLDRWFHHCRPKYSFRRLDDKTTNVSLYPGYNFRYAKYYKENNVEKRTLIKVFGIRFDILVFGTGGKFDIIQLVVYIGSTLSYFGLAAVFIDFLIDTYSSNCCRSHIYPWCKCCQPCVVNEYYYRKKCESIVEPKPTLKYVSFVDESHIRMVNQQLLGRSLQDVKGQEVPRPAMDFTDLSRLPLALHDTPPIPGQPEEIQLLRKEATPRSRDSPVWCQCGSCLPSQLPESHRCLEELCCRKKPGACITTSELFRKLVLSRHVLQFLLLYQEPLLALDVDSTNSRLRHCAYRCYATWRFGSQDMADFAILPSCCRWRIRKEFPKSEGQYSGFKSPY

SEQ ID NO: 4 - protein, 119 aa, Artificial Sequence
  Binding peptide
EVQLLESGGGLVQPGGSLRLSCAASGFTFRNHDMGWVRQAPGKGLEWVSAISGSGGSTYYADSVKGRFTISRDNSKNTLYLQMNSLRAEDTAVYYCAEPKPMDTEFDYRSPGTLVTVSS

SEQ ID NO: 5 - DNA, 357 bp, Artificial Sequence
  coding sequence for binding peptide
gaggtccaactgctggagagtggtgggggtctcgtacaaccggggggatctctcaggctctcatgtgccgcctcaggttttacattccgaaatcatgatatgggctgggtccgccaagctcctgggaaggggttggagtgggtttcagccatctcaggaagcggcggctccacttactacgctgattctgttaaggggcgattcactataagccgagataatagcaagaatactctctatcttcagatgaactcacttcgggcggaagacactgcagtctattattgtgccgaacccaaacccatggacacggagttcgactacagaagccctgggaccttggttacagtgtctagt

SEQ ID NO: 6 - protein, 119 aa, Artificial Sequence
  Binding peptide
EVQLLESGGGLVQPGGSLRLSCAASGFTFRNHDMGWVRQAPGKGLEWVSAISGSGGSTYYADSVKGRFTISRDNSKNTLYLQMNSLRAEDTAVYYCAEPKPMDTEFDYWSPGTLVTVSS

SEQ ID NO: 7 - protein, 119 aa, Artificial Sequence
  Binding peptide
EVQLLESGGGLVQPGGSLRLSCAASGYTFPMKDMGWVRQAPGKGLEWVSAISGSGGGTYYADSVKGRFTISRDNSKNTLYLQMNSLRAEDTAVYYCAEPKPMDTEFDYRSPGTLVTVLE

SEQ ID NO: 8 - protein, 119 aa, Artificial Sequence
  Binding protein
EVQLLESGGGLVQPGGSLRLSCAASGFTFRNHDMGWVRQAPGKGLEWVSAISGSGGSTYYANSVKGRFTISRDNSKNTLYLQMNSLRAEDTAVYYCAEPKPMDTEFDYPSPGTLVTVSS

SEQ ID NO: 9 - protein, 15 aa, Homo sapiens
EPKSCDKTHTCPPCP

SEQ ID NO: 10 - protein, 20 aa, Homo sapiens
NTKVDKTVERKCCVECPPCP

SEQ ID NO: 11 - protein, 70 aa, Homo sapiens
NTKVDKRVELKTPLGDTTHTCPRCPEPKSCDTPPPCPRCPEPKSCDTPPPCPRCPEPKSCDTPPPCPRCP

SEQ ID NO: 12 - protein, 12 aa, Homo sapiens
ESKYGPPCPSCP

SEQ ID NO: 13 - protein, 12 aa, Artificial Sequence
  Linker sequence
ESKYGPPCPPCP

SEQ ID NO: 14 - protein, 21 aa, Homo sapiens
PVPSTPPTPSPSTPPTPSPSC

SEQ ID NO: 15 - protein, 58 aa, Homo sapiens
ESPKAQASSVPTAQPQAEGSLAKATTAPATTRNTGRGGEEKKKEKEKEEQEERETKTP

SEQ ID NO: 16 - protein, 112 aa, Homo sapiens
IAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPD

SEQ ID NO: 17 - protein, 99 aa, Homo sapiens
PTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCA

SEQ ID NO: 18 - protein, 15 aa, Homo sapiens
KHLCPSPLFPGPSKP

SEQ ID NO: 19 - protein, 48 aa, Homo sapiens
AKPTTTPAPRPPTPAPTIASQPLSLRPEACRPAAGGAVHTRGLDFACD

SEQ ID NO: 20 - protein, 45 aa, Homo sapiens
TTTPAPRPPTPAPTIASQPLSLRPEACRPAAGGAVHTRGLDFACD

SEQ ID NO: 21 - protein, 38 aa, Homo sapiens
SVVDFLPTTAQPTKKSTLKKRVCRLPRPETQKGPLCSP

SEQ ID NO: 22 - protein, 36 aa, Homo sapiens
VDFLPTTAQPTKKSTLKKRVCRLPRPETQKGPLCSP

SEQ ID NO: 23 - protein, 36 aa, Homo sapiens
APPRASALPAPPTGSALPDPQTASALPDPPAASALP

SEQ ID NO: 24 - protein, 23 aa, Homo sapiens
DSGQVLLESNIKVLPTWSTPVQP

SEQ ID NO: 25 - protein, 5 aa, Artificial Sequence
  PolyG/S hinge
GGGGS

SEQ ID NO: 26 - protein, 330 aa, Homo sapiens [sequence omitted]

SEQ ID NO: 27 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 28 - protein, 377 aa, Homo sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 29 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 30 - protein, 110 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 31 - protein, 107 aa, Homo sapiens
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 110 aa, Homo sapiens; identical to SEQ ID NO: 30

SEQ ID NO: 33 - protein, 107 aa, Homo sapiens; identical to SEQ ID NO: 31

SEQ ID NO: 34 - protein, 110 aa, Homo sapiens
APELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTK

SEQ ID NO: 35 - protein, 106 aa, Homo sapiens
GQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPG

SEQ ID NO: 36 - protein, 110 aa, Homo sapiens
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAK

SEQ ID NO: 37 - protein, 107 aa, Homo sapiens
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 38 - protein, 12 aa, Artificial Sequence
  Linker domain
ESKYGPPCPPCP

SEQ ID NO: 39 - protein, 119 aa, Artificial Sequence
  Linker sequence
ESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 40 - protein, 228 aa, Artificial Sequence
  Linker sequence
ESKYGPPCPPCPPEFDGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 41 - protein, 30 aa, Artificial Sequence
  CAR sequence
GGGGSGGGGSGGGGSESKYGPPCPPCPDPK

SEQ ID NO: 42 - protein, 164 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 43 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 44 - protein, 180 aa, Homo sapiens
MEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLR

SEQ ID NO: 45 - protein, 171 aa, Homo sapiens
MEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNK

SEQ ID NO: 46 - protein, 112 aa, Artificial Sequence
  Intracellular CAR domain of CD3 zeta
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 47 - protein, 244 aa, Homo sapiens
MDTESNRRANLALPQEPSSVPAFEVLEISPQEVSSGRLLKSASSPPLHTWLTVLKKEQEFLGVTQILTAMICLCFGTVVCSVLDISHIEGDIFSSFKAGYPFWGAIFFSISGMLSIISERRNATYLVRGSLGANTASSIAGGTGITILIINLKKSLAYIHIHSCQKFFETKCFMASFSTEIVVMMLFLTILGLGSAVSLTICGAGEELKGNKVPEDRVYEELNIYSATYSELEDPGEMSPPIDL

SEQ ID NO: 48 - protein, 374 aa, Homo sapiens
MWFLTTLLLWVPVDGQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGLQLPTPVWFHVLFYLAVGIMFLVNTVLWVTIRKELKRKKKWDLEISLDSGHEKKVISSLQEDRHLEEELKCQEQKEEQLQEGVHRKEPQGAT

SEQ ID NO: 49 - protein, 43 aa, Artificial Sequence
  CAR 41BB portion of intracellular domain
VKRGRKKLLYIFKQPFMRPVQTTQEEDGCSCRFPEEEEGGCEL

SEQ ID NO: 50 - protein, 823 aa, Artificial Sequence
  CAR amino acid sequence
MLLLVTSLLLCELPHPAFLLIPEVQLLESGGGLVQPGGSLRLSCAASGFTFRNHDMGWVRQAPGKGLEWVSAISGSGGSTYYADSVKGRFTISRDNSKNTLYLQMNSLRAEDTAVYYCAEPKPMDTEFDYRSPGTLVTVSSESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 51 - protein, 442 aa, Artificial Sequence
  CAR amino acid sequence
MLLLVTSLLLCELPHPAFLLIPEVQLLESGGGLVQPGGSLRLSCAASGFTFRNHDMGWVRQAPGKGLEWVSAISGSGGSTYYADSVKGRFTISRDNSKNTLYLQMNSLRAEDTAVYYCAEPKPMDTEFDYRSPGTLVTVSSESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 52 - DNA, 2478 bp, Artificial Sequence
  Coding Sequence for SEQ ID NO 50
gccaccatgcttctcctggtgacaagccttctgctctgtgagttaccacacccagcattcctcctgatcccagaggtccaactgctggagagtggtgggggtctcgtacaaccggggggatctctcaggctctcatgtgccgcctcaggttttacattccgaaatcatgatatgggctgggtccgccaagctcctgggaaggggttggagtgggtttcagccatctcaggaagcggcggctccacttactacgctgattctgttaaggggcgattcactataagccgagataatagcaagaatactctctatcttcagatgaactcacttcgggcggaagacactgcagtctattattgtgccgaacccaaacccatggacacggagttcgactacagaagccctgggaccttggttacagtgtctagtgaatctaagtacggaccgccctgccccccttgccctggccagcctagagaaccccaggtgtacaccctgcctcccagccaggaagagatgaccaagaaccaggtgtccctgacctgcctggtcaaaggcttctaccccagcgatatcgccgtggaatgggagagcaacggccagcccgagaacaactacaagaccaccccccctgtgctggacagcgacggcagcttcttcctgtactcccggctgaccgtggacaagagccggtggcaggaaggcaacgtcttcagctgcagcgtgatgcacgaggccctgcacaaccactacacccagaagtccctgagcctgagcctgggcaagatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggctcgagggcggcggagagggcagaggaagtcttctaacatgcggtgacgtggaggagaatcccggccctaggatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtga

SEQ ID NO: 53 - DNA, 1333 bp, Artificial Sequence
  Coding sequence for SEQ ID NO 51
cgccaccatgcttctcctggtgacaagccttctgctctgtgagttaccacacccagcattcctcctgatcccagaggtccaactgctggagagtggtgggggtctcgtacaaccggggggatctctcaggctctcatgtgccgcctcaggttttacattccgaaatcatgatatgggctgggtccgccaagctcctgggaaggggttggagtgggtttcagccatctcaggaagcggcggctccacttactacgctgattctgttaaggggcgattcactataagccgagataatagcaagaatactctctatcttcagatgaactcacttcgggcggaagacactgcagtctattattgtgccgaacccaaacccatggacacggagttcgactacagaagccctgggaccttggttacagtgtctagtgaatctaagtacggaccgccctgccccccttgccctggccagcctagagaaccccaggtgtacaccctgcctcccagccaggaagagatgaccaagaaccaggtgtccctgacctgcctggtcaaaggcttctaccccagcgatatcgccgtggaatgggagagcaacggccagcccgagaacaactacaagaccaccccccctgtgctggacagcgacggcagcttcttcctgtactcccggctgaccgtggacaagagccggtggcaggaaggcaacgtcttcagctgcagcgtgatgcacgaggccctgcacaaccactacacccagaagtccctgagcctgagcctgggcaagatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaagg

SEQ ID NO: 54 - protein, 716 aa, Artificial Sequence
  CNA1002 CAR amino acid sequence
MLLLVTSLLLCELPHPAFLLIPEVQLLESGGGLVQPGGSLRLSCAASGFTFRNHDMGWVRQAPGKGLEWVSAISGSGGSTYYADSVKGRFTISRDNSKNTLYLQMNSLRAEDTAVYYCAEPKPMDTEFDYRSPGTLVTVSSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 55 - DNA, 2158 bp, Artificial Sequence
  Coding DNA for SEQ ID NO54
cgccaccatgcttctcctggtgacaagccttctgctctgtgagttaccacacccagcattcctcctgatcccagaggtccaactgctggagagtggtgggggtctcgtacaaccggggggatctctcaggctctcatgtgccgcctcaggttttacattccgaaatcatgatatgggctgggtccgccaagctcctgggaaggggttggagtgggtttcagccatctcaggaagcggcggctccacttactacgctgattctgttaaggggcgattcactataagccgagataatagcaagaatactctctatcttcagatgaactcacttcgggcggaagacactgcagtctattattgtgccgaacccaaacccatggacacggagttcgactacagaagccctgggaccttggttacagtgtctagtgaatctaagtacggaccgccctgccccccttgccctatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggctcgagggcggcggagagggcagaggaagtcttctaacatgcggtgacgtggaggagaatcccggccctaggatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtga

SEQ ID NO: 56 - protein, 932 aa, Artificial Sequence
  CNA1004 CAR amino acid sequence
MLLLVTSLLLCELPHPAFLLIPEVQLLESGGGLVQPGGSLRLSCAASGFTFRNHDMGWVRQAPGKGLEWVSAISGSGGSTYYADSVKGRFTISRDNSKNTLYLQMNSLRAEDTAVYYCAEPKPMDTEFDYRSPGTLVTVSSESKYGPPCPPCPPEFDGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 57 - DNA, 2809 bp, Artificial Sequence
  Coding DNA for SEQ ID NO56
cgccaccatgcttctcctggtgacaagccttctgctctgtgagttaccacacccagcattcctcctgatcccagaggtccaactgctggagagtggtgggggtctcgtacaaccggggggatctctcaggctctcatgtgccgcctcaggttttacattccgaaatcatgatatgggctgggtccgccaagctcctgggaaggggttggagtgggtttcagccatctcaggaagcggcggctccacttactacgctgattctgttaaggggcgattcactataagccgagataatagcaagaatactctctatcttcagatgaactcacttcgggcggaagacactgcagtctattattgtgccgaacccaaacccatggacacggagttcgactacagaagccctgggaccttggttacagtgtctagtgaatctaagtacggaccgccctgccccccttgccctgcccccgagttcgacggcggacccagcgtgttcctgttcccccccaagcccaaggacaccctgatgatcagccggacccccgaggtgacctgcgtggtggtggacgtgagccaggaagatcccgaggtccagttcaattggtacgtggacggcgtggaagtgcacaacgccaagaccaagcccagagaggaacagttccagagcacctaccgggtggtgtctgtgctgaccgtgctgcaccaggactggctgaacggcaaagaatacaagtgcaaggtgtccaacaagggcctgcccagcagcatcgaaaagaccatcagcaaggccaagggccagcctcgcgagccccaggtgtacaccctgcctccctcccaggaagagatgaccaagaaccaggtgtccctgacctgcctggtgaagggcttctaccccagcgacatcgccgtggagtgggagagcaacggccagcctgagaacaactacaagaccacccctcccgtgctggacagcgacggcagcttcttcctgtacagccggctgaccgtggacaagagccggtggcaggaaggcaacgtctttagctgcagcgtgatgcacgaggccctgcacaaccactacacccagaagagcctgagcctgtccctgggcaagatgttctgggtgctggtggtggtgggcggggtgctggcctgctacagcctgctggtgacagtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggctcgagggcggcggagagggcagaggaagtcttctaacatgcggtgacgtggaggagaatcccggccctaggatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtga

SEQ ID NO: 58 - DNA, 1260 bp, Artificial Sequence
  Coding sequence BLIV-CAR short hinge CAR
gaccggcgcctactctagaggagcgcgtcatggccctgcctgtgacagccctgctgctgccactcgctcttctccttcacgccgcaagacccgaagtgcagcttctggagtctggaggtggtttggtgcagcctggcgggtctctcagattgtcatgcgccgcatccggtttcacctttcggaaccatgatatgggttgggtccgccaggccccaggcaagggtcttgagtgggtctccgccatcagcggcagtggcgggtccacatactacgcagactccgtcaaaggcagatttacaatttcacgggataatagtaagaacactctgtacctccagatgaatagtctccgggcggaggacacagctgtgtactattgcgcggagccaaagccaatggatactgagtttgattattggagcccgggaaccctggtgacagtatccagcggcggcggcggctctggcggtgggggtagcggaggcggcggaagcgaatccaaatatggccctccttgtccaccgtgccccgatccaaagttctgggtgctggtggtagtgggtggcgtcctggcctgttattctctgcttgtgacagtcgcgtttatcatcttttgggtccggtctaaacgctctaggttgttgcactccgattacatgaacatgaccccacgccggcctggccctacgcggaagcactaccaaccttacgctcctcccagggatttcgccgcttacaggagccgagatcagagactgccacccgatgcacacaaaccacccggtggtgggtctttcaggaccccaatccaggaggagcaagctgacgcgcattccacccttgccaagataagggtcaaatttagtaggtcagctgacgcgccggcctatcaacagggacagaaccagttgtataatgaactcaatctcggacgacgcgaggagtacgacgtactggataagaggcgcggcagggatcctgaaatgggcggcaagccccggcgaaaaaacccccaggagggactctacaatgagctgcagaaggacaaaatggcagaagcttactccgaaattggaatgaagggcgaaagaaggagagggaaagggcacgatggcctgtatcagggcctgagtaccgccaccaaggacacgtatgatgccctgcatatgcaggcactgccccctagaggaagcggggctacgaatttcagcctcctgaaacaggctggcgacgtggaggaaaatccggggccaggcgaat

SEQ ID NO: 59 - DNA, 1875 bp, Artificial Sequence
  Coding sequence BLIV-CAR long hinge CAR
gaccggcgcctactctagaggagcgcgtcatggctcttcctgtgaccgcattgctgctgccgctggccttgctgctgcatgcagctcggccagaagttcaactgctggagagtggagggggcctcgtgcagccgggcggcagcttgcgcctgtcatgtgcagcaagcgggttcacctttaggaaccacgatatggggtgggtgaggcaggctccgggaaagggtctggaatgggtgagtgccatatcagggagcggaggctccacctactacgcagactccgtgaagggtcggtttacgatttccagagacaattccaagaataccctgtacctgcagatgaactccctccgcgccgaagatacagcagtctactactgtgcagaaccaaaaccaatggatacagaattcgactattggagtcctggaactcttgtcactgtatccagtggaggaggcgagagtaaatatggacctccgtgtccgagttgtcccgcgcctcctgtggccggcccctctgtatttctgtttccacctaagccgaaagatacattgatgattagccgaacaccagaggttacttgtgtggttgttgacgtgagtcaagaggaccctgaggtgcagtttaattggtatgtcgacggagttgaggtgcataacgccaagacgaagccgcgagaggagcagtttaattccacctacagggtcgtatccgttctcactgtccttcaccaggactggctgaatgggaaggagtacaaatgcaaagtgagcaataaaggcctgccgagctccatcgaaaaaaccatttccaaggcaaaaggccaaccccgagagccacaggtctataccctgccaccaagccaggaggaaatgaccaagaatcaggtgagcctcacctgtctggtcaagggcttctacccgtccgacatcgcggtggagtgggagagtaacggacagcctgaaaacaattacaagacaaccccgcctgttttggactctgacggctccttttttctgtactctcggcttaccgtggataagagtagatggcaagaaggcaacgtcttcagctgttccgtgatgcatgaggcgctgcataaccattatacacaaaaaagtctgtccttgagcctgggcaaattttgggtgctggtggtggtggggggtgtcctcgcttgctacagtttgttggtgacagttgcctttattattttttgggtgcgcagtaagcggagtcgcctccttcattccgactatatgaacatgacacctcgccgcccaggcccaacgaggaaacattatcagccatatgcaccacctagagactttgccgcttaccggtcccgagatcaaaggcttccccccgatgcacacaaaccacccggcggtggctcatttcgaacaccaattcaggaagagcaggcagacgcccacagcaccctggccaagatccgggtaaagttcagccgaagtgcagatgcgccggcataccagcagggccagaatcaattgtacaatgagcttaacctcggccgcagagaggagtatgatgtactggataagcggcgcggacgggatcctgagatgggaggaaagcctcggagaaaaaatccccaggaaggactttacaatgagttgcagaaggataagatggccgaagcatattctgaaatcgggatgaaaggtgagcggcggagaggaaaaggccacgacgggctctaccaggggctgagcacagctactaaagatacatacgacgcacttcatatgcaagccctgcctccccgcggaagcggtgccacgaacttttctctcctcaaacaggctggggacgtcgaggaaaatccaggtcccggcgaattcgccaccatgc

SEQ ID NO: 60 - protein, 408 aa, Artificial Sequence
  BLIV-CAR short amino acid sequence
MALPVTALLLPLALLLHAARPEVQLLESGGGLVQPGGSLRLSCAASGFTFRNHDMGWVRQAPGKGLEWVSAISGSGGSTYYADSVKGRFTISRDNSKNTLYLQMNSLRAEDTAVYYCAEPKPMDTEFDYWSPGTLVTVSSGGGGSGGGGSGGGGSESKYGPPCPPCPDPKFWVLVVVGGVLACYSLLVTVAFIIFWVRSKRSRLLHSDYMNMTPRRPGPTRKHYQPYAPPRDFAAYRSRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGP

SEQ ID NO: 61 - protein, 611 aa, Artificial Sequence
  BLIV-CAR long hinge CAR amino acid sequence
MALPVTALLLPLALLLHAARPEVQLLESGGGLVQPGGSLRLSCAASGFTFRNHDMGWVRQAPGKGLEWVSAISGSGGSTYYADSVKGRFTISRDNSKNTLYLQMNSLRAEDTAVYYCAEPKPMDTEFDYWSPGTLVTVSSGGGESKYGPPCPSCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKFWVLVVVGGVLACYSLLVTVAFIIFWVRSKRSRLLHSDYMNMTPRRPGPTRKHYQPYAPPRDFAAYRSRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPGE

SEQ ID NO: 62 - protein, 358 aa, Artificial Sequence
  EGFRt amino acid sequence
RMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 63 - protein, 228 aa, Artificial Sequence
  BLIV-CAR Long linker domain
ESKYGPPCPSCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 64 - protein, 119 aa, Artificial Sequence; identical to SEQ ID NO: 7
  Binding peptide

SEQ ID NO: 65 - protein, 119 aa, Artificial Sequence; identical to SEQ ID NO: 8
  Binding peptide

SEQ ID NO: 66 - protein, 279 aa, Artificial Sequence
  Binding peptide
EVMLVESGGGLVRPGGSLKLSCTASGFTFSGYAMSWVRQTPEKRLEWVATIDSGGYNTYYPDSVKGRFTLSRDNARNTLSLQMSSLRSEDTAIYYCSTSLVEFFDYWGPGTALTVSSAKTTPPSVYPRADAAPTVSIFPPSSNGGGGSGGGGSGGGGSDVLMTQSPLSLPVSLGDQASISCRSSQNIVHSNGNTYLEWYLKKPGQSPKLLIYKVSYRFSGVPDRFSGSGSGTDFTLKISRVEAEDLGVYYCFQGSLVPWTFGGGTKLEIQRADAAPTVS

SEQ ID NO: 67 - protein, 270 aa, Artificial Sequence
  Binding peptide
EVQLLESGGGLVQPGGSLRLSCAASGFTFRNHDMGWVRQAPGKGLEWVSAISGSGGSTYYADSVKGRFTISRDNSKNTLYLQMNSLRAEDTAVYYCAEPKPMDTEFDYPSPGTLVTVSRADAAPTVSIFPPSSNGGGGSGGGGSGGGGSDVLMTQSPLSLPVSLGDQASISCRSSQNIVHSNGNTYLEWYLKKPGQSPKLLIYKVSYRFSGVPDRFSGSGSGTDFTLKISRVEAEDLGVYYCFQGSLVPWTFGGGTKLEIQRADAAPTVS

SEQ ID NO: 68 - protein, 268 aa, Artificial Sequence
  Binding peptide
EVQLLESGGGLVQPGGSLRLSCAASGYTFPMKDMGWVRQAPGKGLEWVSAISGSGGGTYYADSVKGRFTISRDNSKNTLYLQMNSLRAEDTAVYYCAEPKPMDTEFDYRSPGTLVTVLERADAAPTVSIFPPSSNGGGGSGGGGSGGGGSEVQLLESGGGLVQPGGSLRLSCAASGFTFRNHDMGWVRQAPGKGLEWVSAISGSGGSTYYANSVKGRFTISRDNSKNTLYLQMNSLRAEDTAVYYCAEPKPMDTEFDYPSPGTLVTVS

SEQ ID NO: 69 - protein, 31 aa, Artificial Sequence
  Coupling region for multivalent CARs
RADAAPTVSIFPPSSNGGGGSGGGGSGGGGS